(19) United States
(12) Patent Application Publication
(10) Pub. No.: US 2025/0145963 A1
(43) Pub. Date: May 8, 2025

(54) Improved Differentiation Method

(71) Applicant: Koninklijke Nederlandse Akademie Van Wetenschappen, Utrecht (NL)

(72) Inventors: Johannes Carolus Clevers, Utrecht (NL); Joep Beumer, Utrecht (NL)

(73) Assignee: Koninklijke Nederlandse Akademie Van Wetenschappen, Utrecht (NL)

(21) Appl. No.: 18/918,172

(22) Filed: Oct. 17, 2024

Related U.S. Application Data
(63) Continuation of application No. 16/310,933, filed on Dec. 18, 2018, now abandoned.

(30) Foreign Application Priority Data
Jun. 20, 2016 (GB) 1610748.4

(51) Int. Cl. C12N 5/071; A61K 35/38

(52) U.S. Cl. CPC C12N 5/068; A61K 35/38; C12N 2501/11; C12N 2501/155; C12N 2501/415; C12N 2501/42; C12N 2503/02; C12N 2513/00

(57) ABSTRACT

The invention relates to methods and media for differentiating cells, for example for obtaining enteroendocrine cells, and to uses of the cells and organoids obtained by said methods. The invention also relates to methods for modulating hormone expression in enteroendocrine cells and medical uses relating to such methods.

### CROSS REFERENCE TO RELATED APPLICATIONS

This application is a continuation of U.S. application Ser. No. 16/310,933, filed Dec. 18, 2018, which is a national phase entry pursuant to 35 U.S.C. § 371 of International Application No. PCT/EP2017/065101, filed Jun. 20, 2017, which claims the benefit of priority of Great Britain Application No. 1610748.4, filed Jun. 20, 2016, each of which is incorporated by reference herein in its entirety for any purpose.

All documents cited herein are incorporated by reference in their entirety.

### SEQUENCE LISTING

The present application contains a Sequence Listing which has been submitted electronically in XML format. Said XML copy, created on Oct. 16, 2024, is named “01169-0015-01US.xml” and is 25,666 bytes in size. The information in the electronic format of the sequence listing is incorporated herein by reference in its entirety.

### TECHNICAL FIELD

The invention is in the field of cell culture media and methods, in particular culture media and methods for differentiating progenitor cells, e.g. human epithelial stem cells.

### BACKGROUND

There is great interest in culture media and methods for differentiating progenitor cells. Progenitor cells and their differentiated progeny can be used in cellular assays, drug screening, and toxicity assays. Progenitor cells and their differentiated progeny also show promise for cell-based therapies, such as in regenerative medicine for the treatment of damaged tissue. Furthermore, efficient cell culture media are important for providing and maintaining populations of cells for research purposes.

Enteroendocrine cells (EECs) are rare, hormone-secreting cells that can be generated from Lgr5 stem cells (Koo and Clevers (2014) Gastroenterology 147:289-302). Most commonly, subtypes are distinguished based on their secreted hormones and include Somatostatin+ (Sst) DCells, Gastric inhibitory protein+ (Gip) K-Cells, Secretin+ (Sct) S-cells, Cholecystokinin (Cck) I-Cells, Glucagon-like protein 1+ (GLP-1) L-Cells, Neurotensin+ (Nts) N-cells and serotonin producing Enterochromaffin cells (Gunawardene et al. (2011) International journal of experimental pathology 92:219-231). However, a single EEC may express multiple hormones at varying levels, underscoring the high level of heterogeneity (Egerod et al. (2012) Nature cell biology 14:1099-1104). While EECs are believed to play crucial roles in controlling various aspects of intestinal function and of organismal metabolism, their scarcity has posed a hurdle to their in-depth study and exploitation.

Methods for differentiating progenitor cells derived from several tissues (e.g. pancreas, colon, intestinal crypts and stomach) have been described (see WO 2010/090513, WO 2012/014076, WO 2012/168930 and WO 2015/173425). Methods for inducing enterocyte, goblet cell and Paneth cell differentiation were known. There is a need for improved culture media and methods that result in a higher efficiency of differentiation of progenitor cells towards an EEC fate.

### SUMMARY OF THE INVENTION

The invention provides a method for differentiating progenitor cells, wherein said method comprises:
culturing the cells in a differentiation medium comprising a basal medium and further comprising one or more EGFR pathway inhibitors, a Notch inhibitor and one or more Wnt inhibitors.

The invention further provides a differentiation medium comprising a basal medium and further comprising one or more EGFR pathway inhibitors, a Notch inhibitor and one or more Wnt inhibitors.

The invention further provides a method for differentiating intestinal progenitor cells to obtain a population of intestinal cells enriched in enteroendocrine cells, wherein said method comprises:
culturing the intestinal progenitor cells in a differentiation medium of the invention.

The invention further provides a method for culturing epithelial stem cells, preferably to obtain an organoid, wherein said method comprises:
culturing one or more epithelial stem cells in contact with an extracellular matrix in the presence of an expansion medium; and
culturing the one or more expanded epithelial stem cells in a differentiation medium of the invention.

The invention further provides a method for culturing intestine epithelial stem cells, preferably to obtain a differentiated intestine organoid, and wherein said method comprises:
culturing one or more intestine epithelial stem cells in contact with an extracellular matrix in the presence of an expansion medium; preferably wherein the expansion medium comprises a basal medium, and further comprises: a receptor tyrosine kinase ligand (e.g. EGF), a BMP inhibitor (e.g. Noggin) and a Wnt agonist (e.g. Rspondin) and, optionally, valproic acid and a GSK-3 inhibitor (e.g. CHIR99021); and subsequently
culturing the one or more expanded intestine epithelial stem cells in contact with an extracellular matrix in the presence of a differentiation medium of the invention.

The invention further provides an organoid obtainable or obtained by a method of the invention.

The invention further provides an organoid in which at least 30%, at least 40%, at least 50%, at least 60%, at least 70%, at least 80%, at least 90% or at least 99% of the cells express enteroendocrine cell markers.

The invention further provides an organoid of the invention, or a cell derived from said organoid, for use in medicine.

The invention further provides the use of an organoid of the invention, or a cell derived from said organoid, in a drug discovery screen; toxicity assay; diagnostics; research of tissue embryology, cell lineages, and differentiation pathways; research to identify the chemical and/or neuronal signals that lead to the release of the respective hormones; gene expression studies including recombinant gene expression; research of mechanisms involved in tissue injury and repair; research of inflammatory and infectious diseases; studies of pathogenetic mechanisms; or studies of mechanisms of cell transformation and aetiology of cancer.

The invention further provides a pharmaceutical formulation comprising one or more EGFR pathway inhibitors, a Notch inhibitor and one or more Wnt inhibitors.

A method for screening for a therapeutic or prophylactic pharmaceutical drug or cosmetic, wherein the method comprises:
contacting a differentiated organoid of the invention with a candidate molecule (or a library of candidate molecules),
evaluating said organoid for any effects (e.g. any change in the cell, such as a reduction in or loss of proliferation, a morphological change and/or cell death) or a change in organoid (e.g. the organoid size or motility);
identifying the candidate molecule that causes said effects as a potential drug or cosmetic; and optionally
preparing said candidate molecule as pharmaceutical or cosmetic.

The invention further provides a method for inducing quiescence in an Lgr5+ stem cell, wherein said method comprises:
treating the cell with one or more EGFR pathway inhibitors.

The invention further provides a quiescent stem cell population obtained by a method for inducing Lgr5+ progenitor stem cell quiescence of the invention, wherein the cells express Lgr5 and Lef1 and do not express KI67 and M phase marker phospho-Histone H4.

The invention further provides a method of obtaining a population of cells enriched in EECs, wherein the method comprises culturing a population of cells in a differentiation medium of the invention.

The invention further provides a method of obtaining a population of cells enriched in GLP1-secreting EECs, wherein the method comprises culturing a population of cells in a differentiation medium of the invention, wherein the differentiation medium comprises a BMP inhibitor.

The invention further provides a method of obtaining a population of cells enriched in secretin-secreting EECs, wherein the method comprises culturing a population of cells in a differentiation medium of the invention, wherein the differentiation medium comprises a BMP pathway activator.

The invention further provides a BMP inhibitor for use in a method of treating or preventing diabetes mellitus or an associated disease or disorder, wherein the method comprises administering a therapeutically effective amount of the BMP inhibitor to a subject in need thereof.

The invention further provides a BMP activator for use in a method of treating hyperchlorhydria or obesity, wherein the method comprises administering a therapeutically effective amount of the BMP activator to a subject in need thereof.

### DESCRIPTION OF THE DRAWINGS

FIGS. 1A-1G. EGFR Inhibition induces cell cycle exit in intestinal organoids. FIG. 1A, Experimental setup. Organoids were treated with either EGFR (or MEK/ERK inhibitors) or DMSO one week after plating in BME. Samples were collected 1 (d1), 2 (d2), 4 (d4) or 7 (d7) after the treatment. The procedure was repeated from the treatment step onwards for replating experiments.

FIG. 1B, Intestinal organoids after 4 days in control (ENR) or EGFR inhibition (EGFRi). Lgr5^{GFPiresCreER} fluorescence increases following EGFRi treatment. RFP channel is used to display the background. Lower panels are brightfield images. FIG. 1C, Analysis of the cell cycle of intestinal organoids. EdU was administered 1 h prior to the sacrifice. Control (ENR) organoids continuously incorporate EdU (upper panels) and express KI67 (lower panels), while EGFRi treated organoids exit the cell cycle over time. FIG. 1D, Quantification of C. FIG. 1E, HOECHST analysis of the DNA content of control or EGFRi treated organoids. Left-hand peak is EGFRi treated organoids and right-hand peak is control organoids in the ENR medium. Bars on the right show quantification of 3 independent experiments. The top bands of the bars represent cells in G2/M phase, the middle bands of the bars represent cells in S phase and the bottom bands of the bars represent cells in G₀/G₁ phase. FIG. 1F, Analysis of the cell cycle of intestinal organoids following reintroduction of EGF in the culture medium. FIG. 1G, Lgr5^{GFPiresCreER}+ cells exit the cell cycle upon 4 days of EGFRi treatment. Phospho-histone H3 (pH3) staining is used to visualize M phase. The graph at the bottom shows the quantification. DAPI is used to visualize the nuclei. Scale bars=50 μm.

FIGS. 2A-2B. EGFR signalling induced cell cycle exit is mediated by MAPK signalling pathway. FIG. 2A, Histological analysis of ERK phosphorylation following EGFR inhibition. A rapid loss in pERK is gradually over 24 h, but remains low over 48 hours (upper panels). This period coincides with the cell cycle exit of organoids (KI67 staining, lower panels). FIG. 2B, Single inhibition of Mek or Erk as well as simultaneous inhibition of EGFR and ErbB-2 using Afatinib yields similar results to Gefitinib induced EGFR inhibition. EdU is added to the culture medium 1 hour before the sacrifice. Middle panels show endogenous GFP expression from the Lgr5GFPiresCreER allele. Lower panels are brightfield images. DAPI is used to visualize the nuclei. Scale bars=50 μm.

FIGS. 3A-3D. Lineage tracing indicates that qLgr5+ cells are stem cells. FIG. 3A, Experimental setup. Organoids were re-plated in BME either in ENR (control) or EGFRi medium 7 days after dissociation. 4 days after the treatment, recombination was induced with 4′OH Tamoxifen (T) for 16 hours and EGF signalling was restored. Organoids were collected either 4 or 12 days after Tamoxifen induction, or re-plated in ENR for 2 rounds. FIG. 3B, Recombined (YFP+) cells generated CHGA+ EECs, LYZ+ Paneth cells (left panels) or Tuft cells identified by their apical actin and acetylated tubulin dense bundles (right panels). Both active (upper panels) and quiescent (lower panels) Lgr5+ cells were traced. FIG. 3C, Quantification of FIG. 3B. The bottom band in each bar is CBC, the band immediately above the bottom band is EEC, the next band above is Paneth, the next band above is Tuft and the top band in each bar is Rest. FIG. 3D, Recombined (identified by X-Gal staining) active (upper left panel) and quiescent (lower left panel) Lgr5+ cells generate entire organoids displaying multipotency. Recombined Dclk1+ cells (right panels) did not expand in either condition. Scale bars=50 μm.

FIGS. 4A-4F. RNA sequencing identifies key molecular differences between qLgr5+ and aLgr5+ stem cells. FIG. 4A, Hierarchical clustering of the whole transcript of sorted Lgr5+ cells using the Lgr5^{GFPDTR} (Lgr5^{DTR}), Lgr5^{GFPiresCreER} (Lgr5^{GFP}) and Tuft cells using the Dclk1^{GFPiresCreER} (Dclk1) alleles from control (ENR) or EGFR inhibited (EGFRi) conditions. Control organoids were added as a reference. FIG. 4B, Principal component analysis (PCA). FIG. 4C, A volcano plot showing the comparison of active and quiescent Lgr5 cells. X axis shows adjusted p value (q value, in −log 10) and y axis shows fold change (in log 2). Grey dots indicate genes with a false discovery rate (FDR) less than 0.01, black dots depict genes that are not significantly changed. FIG. 4D, Heatmap displaying genes differentially regulated by EGFR inhibition in Lgr5+ cells. Colours indicate z valued of each row (gene). FIG. 4E, Boxplots displaying normalized expression values of marker genes. FIG. 4F, Heatmap displaying k-means clustering of Pearson correlation of the whole transcriptome of individual active and quiescent Lgr5+ cells.

FIGS. 5A-5C. Derivation of a high purity EEC culture. FIG. 5A, Marker analysis of EECs (CHGA) and Paneth cells (LYZ). Organoids were treated for 4 days with Notch inhibitor DAPT (D), inhibitor of Wnt secretion IWP-2 (I), Gefitinib (EGFRi) or a combination of these treatments. DMSO is used as a control. FIG. 5B, Inhibition of Mek signalling (Meki) together with Wnt and Notch signalling pathways similarly increases EEC cell numbers. Dark-grey and light-grey V-shaped arrows point to areas of high SCT and high GIP expression, respectively, in the left half of the figure. Dark-grey and light-grey V-shaped arrows point to areas of high CCK and high SST expression, respectively, in the left half of the figure. Gastric inhibitory protein (GIP), Secretin (SCT), Somatostatin (SST) and cholecystokinin (CCK) positive cell numbers dramatically increase. FIG. 5C, qPCR analysis of EEC related marker gene expression in organoids. EI: EGFRi and MI: Meki. Scale bars=50 μm. Error bars indicate standard deviation.

FIGS. 6A-6D. Single cell transcriptome profiling reveals heterogeneity among induced EECs. FIG. 6A, Heatmap displaying k-means clustering of Pearson correlation of the whole transcriptome of individual live organoid cells from Meki and EGFRi experiments. Numbers indicates clusters. The colours code for Pearson correlation of the whole cellular transcriptome. FIG. 6B, t-SNE map depicting individual cells and marker genes expressed by group of cells. FIG. 6C, t-SNE maps displaying the log 2 transformed colour coded transcript counts of respective genes. FIG. 6D, Heatmap displaying colour-coded transcript counts of respective genes in clusters 2, 5, 6 and 7.

FIGS. 7A-7G. The role of niche signalling pathways in proliferation of Lgr5+ cells. FIG. 7A, FACS plots showing the endogenous Lgr5^{GFPDTR} fluorescence (upper panels, x axis) and KI67^{efluor660} immunostaining (lower panels, x axis). PL3 channel is used (y axis) to discriminate background. Gates shows positive cells with respect to wild type controls. FIG. 7B, Brightfield (upper) and fluorescent (lower) images of control (ENR) and EGFR inhibited (EGFRi) organoids using the Lgr5^{GFPiresCreER} and Rosa-TOP^{CFP} alleles. RFP channel is used to discriminate background. FIG. 7C, Marker gene analysis of Lgr5^{GFPiresCreER}+ cells 4 days after EGFRi treatment. Light-grey and dark-grey V-shaped arrows point to areas of high CHGA and LYZ expression, respectively, in the left-hand image. In the middle and right-hand images, the light-grey V-shaped arrows point to areas of high Phalloidin expression. FIG. 7D, qPCR analysis of marker gene expression in 4 days EGFRi treated organoids. FIG. 7E, Relative number of marker protein positive cells following reintroduction of EGF signalling for 1 (d1), 3 (d3) or 5 (d5) days compared to respective controls (DMSO treated, same days in culture). FIG. 7F, Cell cycle entry of organoids following EGF introduction after repeated cycles of EGF withdrawal (NR) or EGFRi treatment (2×NR+ EGFRi). FIG. 7G, Quantification of F. Scale bars=50 μm. Error bars indicate standard deviation. Key: ENR=EGF, Noggin and R-spondin1; −R=R-spondin1 withdrawal; −N=Noggin withdrawal; EGFRi=inhibition of EGFR signalling (using Gefitinib accompanied by withdrawal of EGF from the culture medium).

FIGS. 8A-8C. Cellular composition of organoids after EGFRi treatment. FIG. 8A, Alcian blue, PAS and Mucin2 (MUC2) staining on paraffin sections of control (ENR) or EGFRi treated organoids. FIG. 8B, 3D reconstruction of whole organoids stained for Paneth cell (LYZ) and EEC (CHGA) markers. Both cell types are more concentrated in EGFRi treated organoids (lower panel) compared to controls. Graph on the right provides the quantification. FIG. 8C, Tuft cell numbers, visualized using the endogenous fluorescence of the Dclk1^{GFPiresCreER} allele, increase after EGFRi treatment (right panel) compared to the controls (left panel). GFP fluorescence does not overlap with EEC (CHGA) or Paneth cell markers (LYZ). Scale bars=50 μm.

FIGS. 9A-9C. Gene ontology (GO) term and single cell analysis of Lgr5+ cell signatures. FIG. 9A, GO term analysis (using Revigo) of genes downregulated after EGFRi treatment in Lgr5 cells. Cell cycle and division related terms as well as small molecule biosynthesis (all related to the cell cycle progression) are significantly higher in active compared to quiescent Lgr5+ cells. x-axis indicates the p-value (−log 10), y axis shows the size of the GO term set. FIG. 9B, t-SNE map displaying the distribution of the sequenced Lgr5+ cells. FIG. 9C, t-SNE maps displaying log 2 transformed color coded transcript levels of Lyz and Dclk1 mRNAs that are used to identify Paneth and Tuft cells, respectively.

FIGS. 10A-10C. Initial analysis of the single cell sequencing of induced EEC organoids. FIG. 10A, t-SNE map displaying the distribution of clusters identified by RaceID. FIG. 10B, t-SNE map comparing the distribution of cells derived from IDEGFRi and IDMeki experiments. FIG. 10C, t-SNE maps displaying log 2 transformed colour coded transcript levels of the Apoa1 mRNA that is used to identify enterocytes.

Summary of intestinal progenitor cell fates. An intestinal progenitor cell can differentiate into a number of cell types, such as an enterocyte, goblet cell, Paneth cell or enteroendocrine cell. It was previously known that Notch activation, coupled with Wnt inhibition promoted enterocyte differentiation. It was also known that Notch inhibition coupled with Wnt activation could promote Paneth cell differentiation. In addition, it was known that Wnt and Notch inhibition could promote Goblet cell differentiation. However, there was previously a lack of understanding regarding how to enhance enteroendocrine cell differentiation.

FIGS. 11A-11B. Composition of EEC culture. Treatment with IDMI differentiation medium resulted in differentiation mainly biased towards enterochromaffin differentiation. The absolute increase in EEC per organoid is mainly enterochromaffin cells. GLP1, CCK, NTS, STT and GIP producing cells are present to a lesser extent.

FIGS. 12A-12H. qPCR results showing messenger RNA levels in different EEC differentiation protocols. Bar graphs show fold mRNA expression of various EEC markers relative to a control. Activation of the BMP pathway selectively enhances secretin messenger RNA at the expense of TAC1 (marker of Enterochromaffin cells). BMP4 was used to activate the BMP pathway and was present at a concentration of 10 g/ml. Basic condition: IDMI (IWP2, DAPT and a MEK inhibitor). MHY1485 is an mTOR activator that was tested at a concentration of 5 μM. Vismogenib is a Hedgehog inhibitor (specifically a Smoothened inhibitor) that was tested at a concentration of 5 M.

FIGS. 13A-13F. Staining for Secretin and GIP in different Enteroendocrine differentiation protocols. Activation of the BMP pathway greatly enhances the number of S cells.

FIGS. 14A-14F. Staining for Serotonin in different Enteroendocrine differentiation protocols. Activation of the BMP pathway decreases the number of Enterochromaffin cells.

FIGS. 15A-15B. qPCR results showing log 2-change in messenger levels of different EEC markers in EEC differentiation protocol compared to standard differentiation medium. EEC differentiation medium greatly enhances the number of EECs in the human organoids, but not of Paneth cells.

FIGS. 16A-16F. CHGA expression in different Enteroendocrine differentiation protocols.

FIG. 17. Triple inhibition of Notch, Wnt and MEK generates cultures enriched in EECs. 30-80% CHGA+ cells per organoid were observed.

FIG. 18. Hormone expression in EEC culture. All different subtypes of EEC are present in the IDMI cultures and express a single hormone. Some cells are CHGA− but positive for hormone.

FIGS. 19A-19B. Hormone secretion by EECs in culture. 2-day culture medium was collected for the −forskolin results. The cultures were then induced with forskolin for 1 hr and the medium was collected for the + forskolin results.

FIGS. 20A-20E. Differential localization of Tac1, GLP1 and Secretin immunoreactive cells in the intestinal crypt and villus. FIGS. 20A-20C, PYY-GLP1 double positive cells are located in the crypt, whereas L-cells in the villus lose expression of GLP1. FIGS. 20D-20E, Enterochromaffin cells in the crypt that express Serotonin, co-express Tac1. Tac1 expression in the villus is lost, where Enterochromaffin cells start co-expressing Secretin.

FIGS. 21A-21C. BMP signaling is a driver of the villus hormone signature. FIG. 21A, Enteroendocrine cell differentiation (Inhibition of Wnt, Notch and MAPK) medium in which BMP signaling is inhibited by the presence of Noggin, generates a hormone signature reminiscent of the crypt: Secretin is absent, and Serotonin+ EECs always co-express Tac1. Exclusion of Noggin and the introduction of BMP4 to this differentiation cocktail, defined as EEC BMP high, greatly reduces GLP1 numbers, as well as Tac1. Single Serotonin+ Enteroendocrine cells, as well as Secretin+ cells, appear in this medium. FIG. 21B, Quantification of FIG. 21A. FIG. 21C, Brightfield image of small intestinal organoids in either BMP low or high EEC differentiation medium. GCG-Venus reporter was used to follow GLP1 positive cells. Although the different differentiation protocols generate morphologically indistinguishable organoids, GLP1 expression is greatly reduced in the background of BMP activation.

FIGS. 22A-22B. Inhibition of BMP signalling in vivo causes an expansion of the GLP1+ compartment, and suppresses Secretin expression. FIG. 22A, 60 hr treatment through oral gavage of mice with LDN193189 causes an expansion of GLP1 numbers. GLP1 is widely expressed by L-cells in the villus, which do not always co-express PYY. FIG. 22B, Secretin is greatly reduced in the villus of BMPR inhibited mice compared to control treated.

### DETAILED DESCRIPTION

Methods for differentiating progenitor cells from a variety of tissues have previously been described in WO 2010/090513, WO 2012/014076, WO 2012/168930 and WO 2015/173425. Methods for promoting differentiation of an intestinal progenitor cell towards an enterocyte, goblet cell or Paneth cell fate have been described. These are summarised in FIG. 11. However, methods and media for enhancing differentiation of an intestinal progenitor cell to an EEC fate have not been previously described. The present inventors have surprisingly found that the combination of a Wnt inhibitor, an EGFR pathway inhibitor and a Notch inhibitor in a differentiation medium enhances the differentiation of progenitor cells to an EEC fate (see Example 2). EECs represent fewer than 1% of the cell of the intestinal epithelium in vivo. However, the inventors' methods and differentiation media were able to generate organoids in which ˜50% of the cells are EECs.

The inventors have also surprisingly found that by modulating the BMP signalling pathway they can modulate EEC phenotype, particularly in relation to expression levels and hormone secretion (see Example 5). The inventors have shown that certain EEC phenotypes can be obtained by activating or inhibiting BMP signalling. The inventors have also shown that these methods can be effective in vivo thus providing promising new uses for BMP activators and inhibitors in therapy.

The inventors also surprisingly found that the presence of an EGFR pathway inhibitor in a culture medium promotes quiescence of Lgr5+ stem cells (see Example 1). In particular, the inventors discovered a new quiescent state where stem cell potential is maintained but proliferation is paused. It was not previously known that stem cell potential and proliferation could be separated.

### Wnt Inhibitors

The differentiation medium of the invention comprises a Wnt inhibitor. Any suitable Wnt inhibitor may be used.

The Wnt signalling pathway when activated typically prevents β-catenin degradation and enhances β-catenin-mediated signalling. This pathway is defined by a series of events that occur when the cell-surface Wnt receptor complex, comprising a Frizzled receptor and LRP5/6 is activated, usually by an extracellular signalling molecule, such as a member of the Wnt family. This results in the activation of Dishevelled family proteins which inhibit a destruction complex of proteins that degrades intracellular β-catenin. The destruction complex is formed of structural components including APC and axin, to which casein kinases CK1α, δ and ε and GSK-3 are recruited. The destruction complex is thought to phosphorylate β-catenin and to expose it to a ubiquitin ligase, β-TrCP. Ubiquitination of the β-catenin then results in its degradation in the proteasome.

The main effector function of β-catenin is in the nucleus, where it regulates transcription through interaction with various transcription factors, including the TCF/LEF family transcription factors (e.g. Tcf-1, Tcf-3, Tcf-4 and Lef1).

The Wnt pathway is highly regulated. For instance, Wnt signalling is enhanced when Rspondin binds to its receptors (Lgr4, Lgr5 and/or Lgr6). However, two transmembrane E3 ubiquitin ligases, Rnf43 and Znrf3, have been shown to remove Rspondin receptors (e.g. Lgr4, Lgr5 and/or Lgr6) from the cell surface (see, e.g., de Lau et al. 2016). Rspondins are vertebrate-specific Wnt-enhancing agents. In addition, the binding of Dishevelled family proteins to the Frizzled receptor can be inhibited by Dapper family proteins (e.g. Dapper1 and Dapper3). Furthermore, the activity of the destruction complex is thought to be partly regulated by the phosphorylation status of APC, axin and GSK-3. For example, dephosphorylation of APC or axin by phosphatases (e.g. serine/threonine phosphatases such as PP1, PP2C or PP2A) may inhibit β-catenin degradation. In addition, phosphorylation of GSK-3 by kinases (e.g. p38 MAPK, PKA, PKB, PKC, p90RSK or p70S6K) may inhibit GSK-3 activity and so inhibit β-catenin degradation.

The stability of the destruction complex is thought to be partly regulated by two PARPs, Tankyrases 1 and 2. Poly(ADP-ribosyl)ation of axin and auto-poly(ADP-ribosyl)ation by these Tankyrases may promote deoligomerisation of the destruction complex.

In the nucleus, Dishevelled family proteins can form a complex with the histone deacetylase SIRT1, which supports the transcription of Wnt target genes.

A protein that is thought to be key to the secretion of Wnt is the multipass membrane protein Porcupine (Porc), the loss of which results in Wnt accumulating in the endoplasmic reticulum.

The Wnt signalling pathway can be inhibited at many levels and Wnt inhibitors are reviewed in detail in Voronkov and Krauss (2013) Current Pharmaceutical Design 19:634-664.

A Wnt inhibitor is defined as an agent that inhibits TCF/LEF-mediated transcription in a cell or in a population of cells. Accordingly, Wnt inhibitors suitable for use in the invention include:
(1) inhibitors of Wnt secretion (e.g. inhibitors of Porc, such as LGK974, IWP-1 or IWP-2),
(2) competitive and non-competitive inhibitors of the interaction between Wnt or Rspondin and their respective receptors (e.g. OMP-18R5, OMP54F28),
(3) factors that promote the degradation of components of the Wnt receptor complex, such as LRP (e.g. niclosamide) and factors that promote the degradation of Rspondin receptors, such as Znrf3 and/or Rnf43 or factors that activate Znrf3 and/or Rnf43,
(4) inhibitors of Dishevelled family proteins, such as inhibitors that reduce the binding of Dishevelled family proteins to Frizzled receptors and/or components of the destruction complex (e.g. Dapper family proteins, FJ9, sulindac, 3289-8625, J01-017a, NSC668036) or inhibitors that downregulate the expression of Dishevelled family proteins (e.g. niclosamide),
(5) factors that promote destruction complex activity, including (a) inhibitors of phosphatases (e.g. PP1, PP2A and/or PP2C) that dephosphorylate components of the destruction complex, such as axin and/or APC (e.g. okadaic acid or tautomycin) and (b) inhibitors of kinases (e.g. p38 MAPK, PKA, PKB, PKC, p90RSK or p70S6K) that phosphorylate GSK-3 (e.g. SB239063, SB203580 or Rp-8-Br-cAMP),
(6) inhibitors of the deoligomerisation of the destruction complex, such as inhibitors of Tankyrases 1 and/or 2 (e.g. XAV939, IWR1, JW74, JW55, 2-[4-(4-fluorophenyl)piperazin-1-yl]-6-methylpyrimidin-4(3H)-one or PJ34), and
(7) inhibitors of β-catenin target gene expression, including inhibitors of the β-catenin:TCF/Lef transcription complex, such as inhibitors that disrupt the β-catenin:TCF-4 complex (e.g. iCRT3, CGP049090, PKF118310, PKF115-584, ZTM000990, PNU-74654, BC21, iCRT5, iCRT14 or FH535) and inhibitors of the histone deacetylase SIRT1 (e.g. cambinol).

The differentiation medium of the invention comprises a Wnt inhibitor. Any suitable Wnt inhibitor may be used as described in (1)-(7) above. For instance, in one preferred embodiment, the Wnt inhibitor is an inhibitor of Wnt secretion, such as a Porc inhibitor, e.g. selected from IWP-2, IWP-1 and LGK974. In another preferred embodiment, the Wnt inhibitor is an inhibitor of β-catenin target gene expression, for example, an inhibitor of the β-catenin:TCF/Lef transcription complex or an inhibitor of the histone deacetylase SIRT1 (e.g. cambinol). In some embodiments, the inhibitor of the β-catenin:TCF/Lef transcription complex is an inhibitor that disrupts the β-catenin:TCF-4 complex, for example an inhibitor selected from iCRT3, CGP049090, PKF118310, PKF115-584, ZTM000990, PNU-74654, BC21, iCRT5, iCRT14 and FH535.

In some embodiments, the Wnt inhibitor is selected from IWP-2, OMP-18R5, OMP54F28, LGK974, 3289-8625, FJ9, NSC 668036, IWR1 and XAV939.

In some embodiments, the Wnt inhibitor is selected from iCRT3, PFK115-584, CGP049090, iCRT5, iCRT14 and FH535.

In some embodiments, the Wnt inhibitor is one of the compounds listed in Table 1 below.

TABLE 1
Wnt inhibitors

| Structure | Compound | Target |
|---|---|---|
| 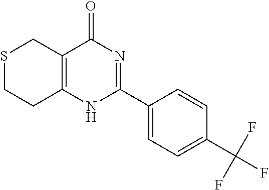 [SMILES: O=c1nc(-c2ccc(C(F)(F)F)cc2)[nH]c2c1CSCC2] | XAV939 | Tankyrases 1, 2 |
| 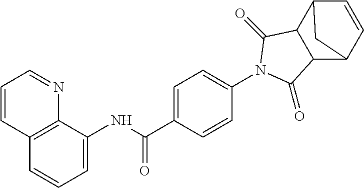 [SMILES: O=C(Nc1cccc2cccnc12)c1ccc(N2C(=O)C3C4C=CC(C4)C3C2=O)cc1] | IWR1 | Tankyrases 1, 2 |
| 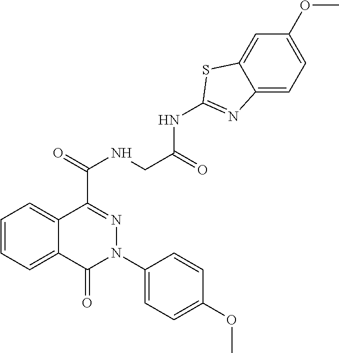 [SMILES: COc1ccc(-n2nc(C(=O)NCC(=O)Nc3nc4ccc(OC)cc4s3)c3ccccc3c2=O)cc1] | IWP-1 | Porcupine |
| 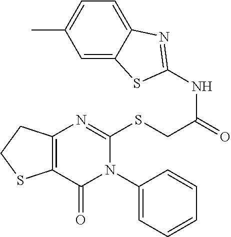 [SMILES: Cc1ccc2nc(NC(=O)CSc3nc4c(c(=O)n3-c3ccccc3)SCC4)sc2c1] | IWP-2 | Porcupine |
| 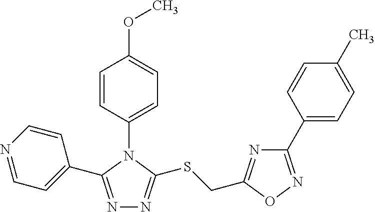 [SMILES: COc1ccc(-n2c(SCc3nc(-c4ccc(C)cc4)no3)nnc2-c2ccncc2)cc1] | JW74 | Tankyrases 1, 2 |
| 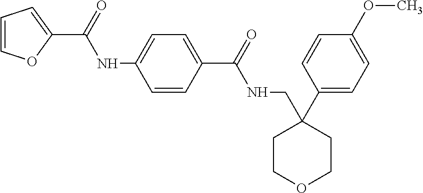 [SMILES: COc1ccc(C2(CNC(=O)c3ccc(NC(=O)c4ccco4)cc3)CCOCC2)cc1] | JW55 | Tankyrases 1, 2 |
| 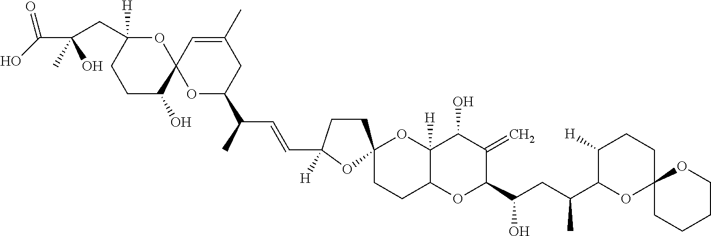 | Okadaic acid | PP2A phosphatase |
| 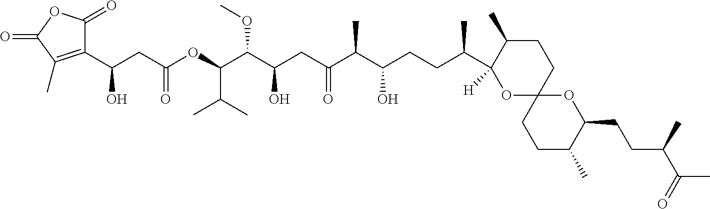 [SMILES: CO[C@H]([C@H](O)CC(=O)[C@@H](C)[C@@H](O)CC[C@@H](C)[C@@H]1OC2(CC[C@@H](C)[C@H](CC[C@@H](C)C(C)=O)O2)CC[C@@H]1C)[C@H](OC(=O)C[C@@H](O)C1=C(C)C(=O)OC1=O)C(C)C] | Tautomycin | PP1 phosphatase |
| 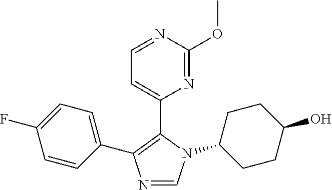 [SMILES: COc1nccc(-c2c(-c3ccc(F)cc3)ncn2[C@H]2CC[C@H](O)CC2)n1] | SB239063 | p38 MAPK |
| 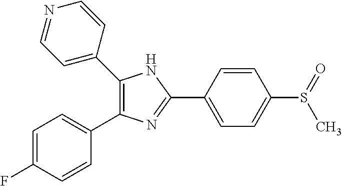 [SMILES: CS(=O)c1ccc(-c2nc(-c3ccc(F)cc3)c(-c3ccncc3)[nH]2)cc1] | SB203580 | p38 MAPK |
| 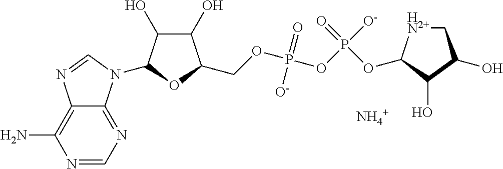 | ADP-HPD | PARG |
| 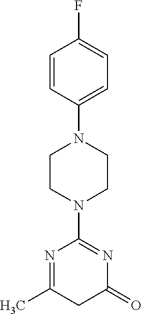 [SMILES: CC1=NC(N2CCN(c3ccc(F)cc3)CC2)=NC(=O)C1] | 2-[4-(4- fluorophenyl) piperazin-1-yl]- 6- methylpyrimidin- 4(3H)-one | Tankyrases 1, 2 |
| 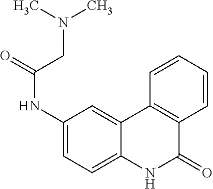 [SMILES: CN(C)CC(=O)Nc1ccc2[nH]c(=O)c3ccccc3c2c1] | PJ34 | Tankyrases 1, 2 |
| 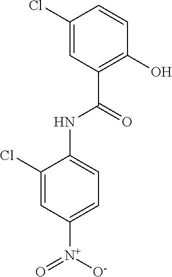 [SMILES: O=C(Nc1ccc([N+](=O)[O-])cc1Cl)c1cc(Cl)ccc1O] | Niclosamide | Down- regulates Dvl-2, triggers LRP6 degradation |
| 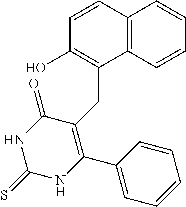 [SMILES: O=c1[nH]c(=S)[nH]c(-c2ccccc2)c1Cc1c(O)ccc2ccccc12] | Cambinol | SIRT1 |
| 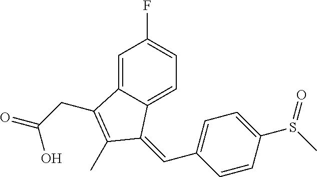 [SMILES: CC1=C(CC(=O)O)c2cc(F)ccc2/C1=C\c1ccc(S(C)=O)cc1] | Sulindac | PDZ domain of Dishevelled |
| 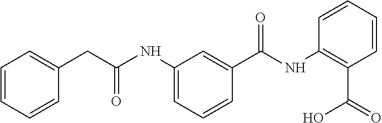 [SMILES: O=C(Cc1ccccc1)Nc1cccc(C(=O)Nc2ccccc2C(=O)O)c1] | 3289-8625 | Dishevelled |
| 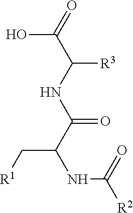 | Scaffold A for series of analogs | Dishevelled |
| 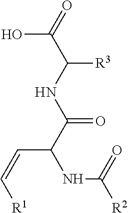 | Scaffold B for series of analogs | Dishevelled |
| 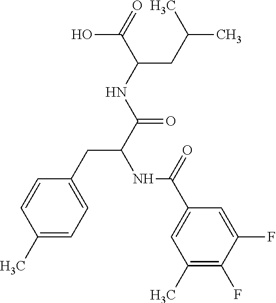 [SMILES: Cc1ccc(CC(NC(=O)c2cc(C)c(F)c(F)c2)C(=O)NC(CC(C)C)C(=O)O)cc1] | J01-017a | Dishevelled |
| 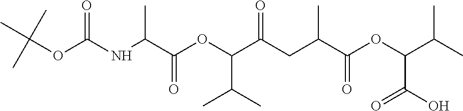 [SMILES: CC(CC(=O)C(OC(=O)C(C)NC(=O)OC(C)(C)C)C(C)C)C(=O)OC(C(=O)O)C(C)C] | NSC668036 | Dishevelled |
| 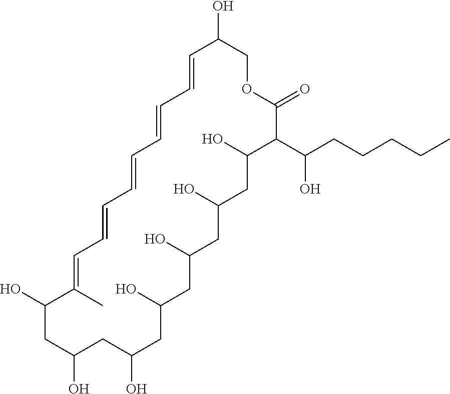 [SMILES: CCCCCC(O)C1C(=O)OCC(O)/C=C/C=C/C=C/C=C/C=C(\C)C(O)CC(O)CC(O)CC(O)CC(O)CC(O)CC1O] | Filipin | Caveolin- mediated endocytosis |
| 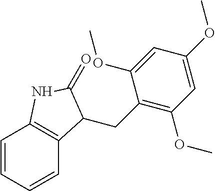 [SMILES: COc1cc(OC)c(CC2C(=O)Nc3ccccc32)c(OC)c1] | IC261 | CK1ε/δ |
| 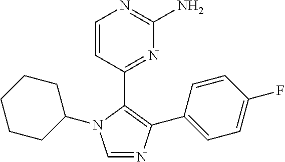 [SMILES: Nc1nccc(-c2c(-c3ccc(F)cc3)ncn2C2CCCCC2)n1] | PF670462 | CK1δ and CK1ε |
| 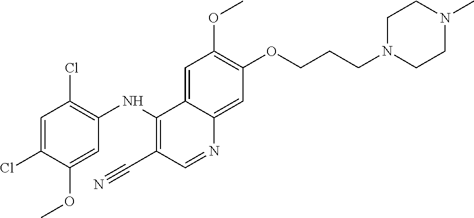 [SMILES: COc1cc(Nc2c(C#N)cnc3cc(OCCCN4CCN(C)CC4)c(OC)cc23)c(Cl)cc1Cl] | Bosutinib | Src kinase |
| 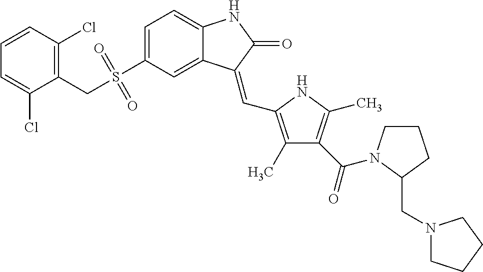 [SMILES: Cc1[nH]c(/C=C2\C(=O)Nc3ccc(S(=O)(=O)Cc4c(Cl)cccc4Cl)cc32)c(C)c1C(=O)N1CCCC1CN1CCCC1] | PHA665752 | c-Met |
| 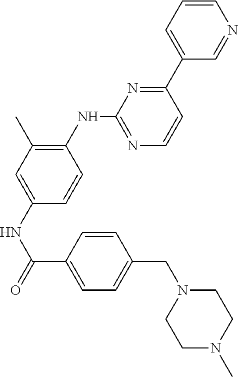 [SMILES: Cc1cc(NC(=O)c2ccc(CN3CCN(C)CC3)cc2)ccc1Nc1nccc(-c2cccnc2)n1] | Imatinib | Different tyrosine kinases |
| 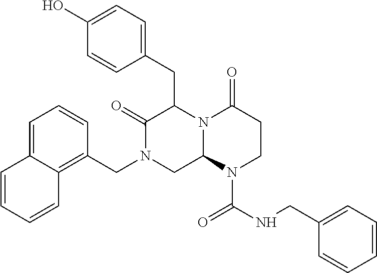 [SMILES: O=C1C(Cc2ccc(O)cc2)N2C(=O)CCN(C(=O)NCc3ccccc3)[C@H]2CN1Cc1cccc2ccccc12] | ICG-001 | CREB binding protein (CBP) |
| 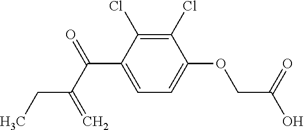 [SMILES: C=C(CC)C(=O)c1ccc(OCC(=O)O)c(Cl)c1Cl] | Ethacrynic acid | Lef-1 |
| 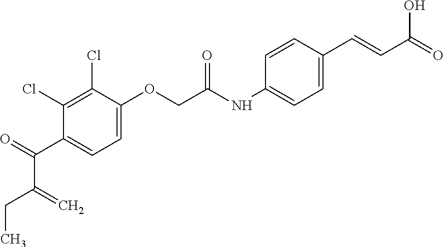 [SMILES: C=C(CC)C(=O)c1ccc(OCC(=O)Nc2ccc(/C=C/C(=O)O)cc2)c(Cl)c1Cl] | Ethacrynic acid derivative | Lef-1 |
| 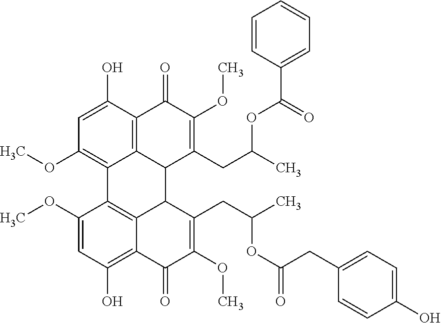 [SMILES: COC1=C(CC(C)OC(=O)Cc2ccc(O)cc2)C2c3c(c(O)cc(OC)c3-c3c(OC)cc(O)c4c3C2C(CC(C)OC(=O)c2ccccc2)=C(OC)C4=O)C1=O] | PKF115-584 | β-catenin |
| 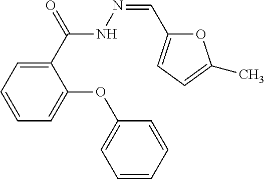 [SMILES: Cc1ccc(/C=N\NC(=O)c2ccccc2Oc2ccccc2)o1] | PNU-74654 | β-catenin |
| 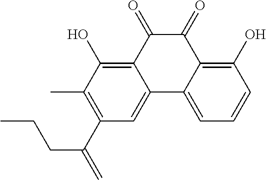 [SMILES: C=C(CCC)c1cc2c(c(O)c1C)C(=O)C(=O)c1c(O)cccc1-2] | PKF118-744 | β-catenin |
| 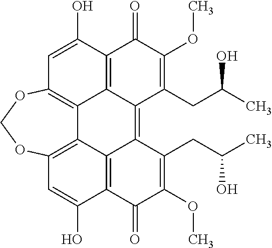 [SMILES: COc1c(C[C@H](C)O)c2c3c(C[C@H](C)O)c(OC)c(=O)c4c(O)cc5c(c6c(cc(O)c(c1=O)c62)OCO5)c43] | CGP049090 | β-catenin |
| 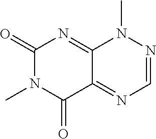 | PKF118-310 | β-catenin |
| 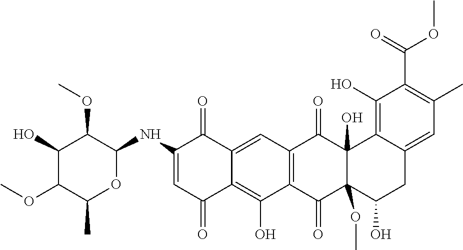 | ZTM000990 | β-catenin |
| 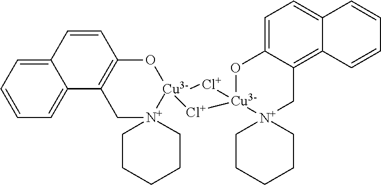 | BC21 | β-catenin |
| 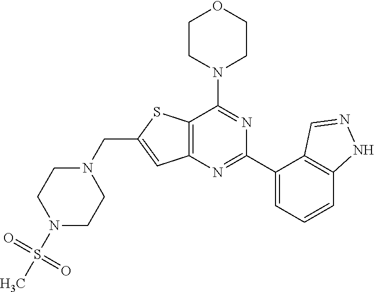 [SMILES: CS(=O)(=O)N1CCN(Cc2cc3nc(-c4cccc5[nH]ncc45)nc(N4CCOCC4)c3s2)CC1] | GDC-0941 | PI3K |
| 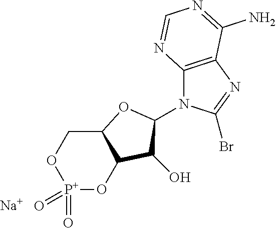 | Rp-8-Br-cAMP | PKA |

In some embodiments, a differentiation medium of the invention comprises one or more of any of the Wnt inhibitors listed in table 1.

The Wnt inhibitor is preferably added to the media in an amount effective to inhibit a Wnt activity in a cell by at least 10%, more preferred at least 20%, more preferred at least 30%, more preferred at least 50%, more preferred at least 70%, more preferred at least 90%, more preferred 100%, relative to a level of said Wnt activity in the absence of said molecule, as assessed in the same cell type. As is known to a skilled person, Wnt activity can be determined by measuring the transcriptional activity of Wnt, for example by pTOPFLASH and pFOPFLASH Tcf luciferase reporter constructs (Korinek et al. (1997) Science 275:1784-1787). New Wnt inhibitors can therefore easily be identified by a skilled person using an assay known in the art.

In some embodiments, the differentiation medium of the invention comprises a Wnt inhibitor at a concentration of 0.01-150 μM, 0.1-150 μM, 0.5-100 μM, 0.1-100 μM, 0.5-50 μM, 1-100 μM or 10-80 μM, 1-20 μM or 1-5 μM.

In some embodiments, the differentiation medium of the invention comprises IWP-2 at a concentration of 0.01-150 μM, 0.1-100 μM, 0.5-50 μM, 1-20 μM or 1-5 μM. For example, in some embodiments, the differentiation medium of the invention comprises IWP-2 at a concentration of about 1.5 μM.

In some embodiments, the differentiation medium does not comprise a Wnt agonist that binds and activates the Wnt receptor complex including any and all of the Wnt family proteins and Rspondin.

In other embodiments, the differentiation medium further comprises a Wnt agonist, such as R-spondin 1-4 or a biologically active fragment or variant thereof. As described above R-spondins enhance Wnt signalling at receptors at the cell surface. The inventors have shown that removal of R-spondin from the EEC differentiation medium decreases the efficiency of EEC differentiation (see Example 5). It is hypothesised that some Wnt signalling may be required to direct the cells towards the secretory (rather than absorptive) lineage. Therefore, in some embodiments the differentiation medium comprises both a Wnt agonist (particularly an R-spondin) and a Wnt inhibitor. For example, in some embodiments, the differentiation medium comprises an R-spondin and a Porc inhibitor, such as IWP-2. In some embodiments, the R-spondin is used at a final concentration of between 1 and 1000 ng/ml, between 50 and 1000 ng/ml or between 100 and 1000 ng/ml. In some embodiments, the R-spondin is used at a final concentration of between 0.1 and 100 μg/ml, between 0.1 and 50 μg/ml, between 0.1 and 20 μg/ml, between 0.1 and 10 μg/ml, between 0.1 and 5 μg/ml, between 0.5 and 100 μg/ml, between 0.5 and 50 μg/ml, between 0.5 and 20 μg/ml, between 0.5 and 10 μg/ml, between 0.5 and 5 μg/ml, between 1 and 10 μg/ml, or between 1 and 5 μg/ml. In some embodiments the R-spondin is used at a final concentration of at least 1 ng/ml, at least 50 ng/ml, at least 100 ng/ml, at least 500 ng/ml or at least 1 μg/ml. In some embodiments the R-spondin is used at a final concentration of about 100 ng/ml. In some embodiments the R-spondin is used at a final concentration of about 1 μg/ml.

### EGFR Pathway Inhibitors

The differentiation medium of the invention comprises an EGFR pathway inhibitor. Any suitable inhibitor as defined herein may be used.

Epidermal growth factor receptor (EGFR), also known as ErbB1 or HER1, is a cell surface receptor for members of the epidermal growth factor (EGF) family of extracellular protein ligands. EGFR belongs to the HER family of receptors which comprise four related proteins (EGFR(HER1/ErbB1), ErbB2(HER2), ErbB3(HER3) and ErbB4(HER4)). The HER receptors are known to be activated by binding to different ligands, including EGF, TGFA, heparin-binding EGF-like growth factor, amphiregulin, betacellulin, and epiregulin. After a ligand binds to the extracellular domain of the receptor, the receptor forms functionally active dimers (EGFR-EGFR (homodimer) or EGFR-HER2, EGFR-HER3, EGFR-HER4 (heterodimer)). Dimerization induces the activation of the tyrosine kinase domain, which leads to autophosphorylation of the receptor on multiple tyrosine residues. This leads to recruitment of a range of adaptor proteins (such as SHC, GRB2) and activates a series of intracellular signalling cascades to affect gene transcription.

The pathways mediating downstream effects of EGFR have been well studied and three major signalling pathways have been identified. The first pathway involves RAS-RAF-MAPK pathway, where phosphorylated EGFR recruits the guanine-nucleotide exchange factor via the GRB2 and Shc adapter proteins, activating RAS and subsequently stimulating RAF and the MAP kinase pathway to affect cell proliferation, tumor invasion, and metastasis. Activated RAS activates the protein kinase activity of RAF kinase. RAF kinase phosphorylates and activates MEK (also known as MAP2K or MAPKK), which phosphorylates and activates a MAP kinase (also known as an ERK, an extracellular signal-regulated kinase). The second pathway involves PI3K/AKT pathway, which activates the major cellular survival and anti-apoptosis signals via activating nuclear transcription factors such as NFKB. The third pathway involves JAK/STAT pathway which is also implicated in activating transcription of genes associated with cell survival. EGFR activation may also lead to phosphorylation of PLCG and subsequent hydrolysis of phosphatidylinositol 4,5 biphosphate (PIP2) into inositol 1,4,5-triphosphate (IP3) and diacylglycerol (DAG), resulting in activation of protein kinase C (PRKC) and CAMK.

EGFR inhibitors, such as anti-EGFR monoclonal antibodies and small-molecule EGFR tyrosine kinase inhibitors, are available. Some anti-EGFR antibodies, such as cetuximab and panitumumab, bind to the extracellular domain of EGFR monomer and compete for receptor binding by the endogenous ligands; in this way they block ligand-induced receptor activation. Some small molecule EGFR inhibitors, such as erlotinib, gefitinib and lapatinib, compete with ATP to bind the kinase domain of EGFR which in turn inhibits EGFR autophosphorylation and downstream signalling.

One or more EGFR pathway inhibitors may be used, for example, 2, 3, 4 or more.

The EGFR pathway inhibitor is preferably added to the media in an amount effective to inhibit an EGFR pathway activity in a cell by at least 10%, more preferred at least 20%, more preferred at least 30%, more preferred at least 50%, more preferred at least 70%, more preferred at least 90%, more preferred 100%, relative to a level of said EGFR pathway activity in the absence of said molecule, as assessed in the same cell type. As is known to a skilled person, EGFR pathway activity can be measured in a variety of ways. For example, an assay for monitoring EGFR activity and inhibitor sensitivities is described in Ghosh et al. (2013) Assay and Drug Development Technologies 11(1):44-51. This particular assay involves peptide substrates that are covalently immobilized to magnetic beads. After kinase reactions, the beads are washed and phosphorylation of the peptides is detected by chemifluorescence using an HRP-conjugated primary antibody against phosphorylated tyrosine. The fluorescence intensity measured is directly proportional to substrate phosphorylation, which in turn is proportional to EGFR kinase activity. This assay could also be used to screen for inhibitors of other kinases in the EGFR pathway (e.g. RAS, RAF, MEK or ERK). An alternative method for assaying kinase activity involves detecting incorporation of terminal phosphate from P³²-labelled ATP. New EGFR pathway inhibitors can therefore easily be identified by a skilled person using an assay known in the art.

In some embodiments, the EGFR pathway inhibitor is an EGFR inhibitor that inhibits EGFR kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is a RAS inhibitor that inhibits RAS kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an RAF inhibitor that inhibits RAF kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an MEK inhibitor that inhibits MEK kinase activity by at least 10%, more preferred at least 20%, more preferred at least 30%, more preferred at least 50%, more preferred at least 70%, more preferred at least 90%, more preferred 100%.

In some embodiments, the EGFR pathway inhibitor is an ERK inhibitor that inhibits ERK kinase activity by at least 10%, more preferred at least 20%, more preferred at least 30%, more preferred at least 50%, more preferred at least 70%, more preferred at least 90%, more preferred 100%.

In some embodiments, EGF is present in the differentiation medium at a concentration of less than 1 mM. In a preferred embodiment, EGF is not present in the differentiation medium.

In some embodiments, the EGFR pathway inhibitor is an EGFR inhibitor, such as Gefitinib (Santa Cruz Biotechnology), AG-18, AG-490 (tyrphostin B42), AG-1478 (tyrphostin AG-1478), AZ5104, AZD3759, brigatinib, erlotinib, cetuximab, CL-387785 (EKI-785), CNX-2006, icotinib, necitumumab, osimertinib (AZD9291), OSI-420, PD153035 HCl, PD168393, pelitinib (EKB-569), rociletinib (CO-1686, AVL-301), TAK-285, tyrphostin 9, vandetanib, WHI-P154, WZ3146, WZ4002, WZ8040, panitumumab, zalutumumab, nimotuzumab or matuzumab. In some embodiments, the EGFR inhibitor binds to the extracellular domain of EGFR monomer and competes for receptor binding by EGF. In some embodiments, the EGFR inhibitor competes with ATP to bind the kinase domain of EGFR. One or more EGFR inhibitors may be used, for example, 2, 3, 4 or more.

In some embodiments, the EGFR pathway inhibitor is an EGFR and ErbB-2 inhibitor, such as Afatinib (Selleckchem), afatinib dimaleate, AC480 (BMS-599626), AEE788 (NVP-AEE788), AST-1306, canertinib, CUDC-101, dacomitinib, lapatinib, neratinib, poziotinib (HM781-36B), sapitinib (AZD8931) or varlitinib. One or more EGFR and ErbB-2 inhibitors may be used, for example, 2, 3, 4 or more.

In some embodiments, the EGFR pathway inhibitor is an inhibitor of the RAS-RAF-MAPK pathway. In some embodiments, the EGFR pathway inhibitor is an inhibitor of the PI3K/AKT pathway. In some embodiments, the EGFR pathway inhibitor is an inhibitor of the JAK/STAT pathway.

In some embodiments, the EGFR pathway inhibitor is a RAF inhibitor, such as GW5074, ZM 336372, NVP-BHG712, TAK-632, darafenib (GSK2118436), sorafenib, sorafenib tosylate, PLX-4720, AZ 628, CEP-32496 or vemurafenib (PLX4032, RG7204).

In some embodiments, the EGFR pathway inhibitor is an MEK inhibitor, such as PD0325901 (Sigma Aldrich). In some embodiments, the EGFR pathway inhibitor is an ERK inhibitor, such as SCH772984 (Selleckchem).

In some embodiments, the EGFR pathway inhibitor is used at a concentration of 0.01-200 μM, 0.01-100 μM, 0.1-50 μM, 0.1-20 μM, 1-100 μM, 1-50 μM, 1-30 μM, 5-100 μM, 5-50 μM or 5-20 μM. For example, in some embodiments, the differentiation medium comprises: (i) Gefitinib at a concentration of about 5 μM, (ii) Afatinib at a concentration of about 10 μM, (iii) PD0325901 at a concentration of about 5 μM, or (iv) SCH772984 at a concentration of about 10 μM.

### Notch Inhibitor

The differentiation medium comprises a Notch inhibitor. Any suitable Notch inhibitor may be used.

Notch is a transmembrane surface receptor that can be activated through multiple proteolytic cleavages, one of them being cleavage by a complex of proteins with protease activity, termed gamma-secretase. Gamma-secretase is a protease that performs its cleavage activity within the membrane. Gamma-secretase is a multicomponent enzyme and is composed of at least four different proteins, namely, presenilins (presenilin 1 or 2), nicastrin, PEN-2 and APH-I. Presenilin is the catalytic centre of gamma-secretase. On ligand binding the Notch receptor undergoes a conformational change that allows ectodomain shedding through the action of an ADAM protease which is a metalloprotease. This is followed immediately by the action of the gamma-secretase complex which results in the release of the Notch intracellular domain (NICD). NICD translocates to the nucleus where it interacts with CSL (C-promoter-binding factor/recombinant signal-sequence binding protein Jx/Supressor-of-Hairless/Lagl). The binding of NICD converts CSL from a transcriptional repressor to an activator which results in the expression of Notch target genes.

In some embodiments, the Notch inhibitor is an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases.

In some embodiments the Notch inhibitor is a gamma-secretase inhibitor, for example DAPT, dibenzazepine (DBZ), benzodiazepine (BZ) or LY-411575. One or more Notch inhibitors may be used, for example, 2, 3, 4 or more.

In some embodiments, the Notch inhibitor (e.g. DAPT) is used at a concentration of 0.001-200 mM, 0.01-100 mM, 0.1-50 mM, 0.1-20 mM, 0.5-10 mM or 0.5-5 mM. For example, in some embodiments, the differentiation medium comprises DAPT at a concentration of about 1 mM.

### Basal Medium

A differentiation medium that is used in a method of the invention comprises a basal medium. The basal medium is any suitable basal medium for animal or human cells, subject to the limitations provided herein.

Basal media for animal or human cell culture typically contain a large number of ingredients, which are necessary to support maintenance of the cultured cells. Suitable combinations of ingredients can readily be formulated by the skilled person, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature and above. In some embodiments, the culture medium is further supplemented with one or more standard cell culture ingredient, for example selected from amino acids, vitamins, lipid supplements, inorganic salts, a carbon energy source, and a buffer.

The skilled person will understand from common general knowledge the types of culture media that might be used as the basal medium in the differentiation medium of the invention. Potentially suitable cell culture media are available commercially, and include, but are not limited to, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

For example, the basal medium may be selected from DMEM/F12 and RPMI 1640 supplemented with glutamine, insulin, penicillin/streptomycin and transferrin. In a further preferred embodiment, Advanced DMEM/F12 or Advanced RPMI is used, which is optimized for serum free culture and already includes insulin. In this case, said Advanced DMEM/F12 or Advanced RPMI medium is preferably supplemented with glutamine and penicillin/streptomycin. AdDMEM/F12 (Invitrogen) supplemented with N2 and B27 is also preferred. Preferably, the basal medium is Advanced DMEM/F12. More preferably, the basal medium comprises Advanced DMEM/F12, glutamine and B27.

In some embodiments, the basal medium comprises Advanced DMEM/F12, HEPES, penicillin/streptomycin, Glutamine, N-Acetylcysteine and B27.

In some embodiments, the basal culture medium comprises or consists of Advanced DMEM/F12 supplemented with penicillin/streptomycin, 10 mM HEPES, Glutamax, B27 (all from Life Technologies, Carlsbad, CA) and about 1 mM N-acetylcysteine (Sigma).

It is furthermore preferred that said basal culture medium is supplemented with a purified, natural, semi-synthetic and/or synthetic growth factor and does not comprise an undefined component such as fetal bovine serum or fetal calf serum. Various different serum replacement formulations are commercially available and are known to the skilled person. Where a serum replacement is used, it may be used at between about 1% and about 30% by volume of the medium, according to conventional techniques.

The differentiation medium used in the invention may comprise serum, or may be serum-free and/or serum-replacement free, as described elsewhere herein. Culture media and cell preparations are preferably GMP processes in line with standards required by the FDA for biologics products and to ensure product consistency.

A differentiation medium of the invention will normally be formulated in deionized, distilled water. A differentiation medium of the invention will typically be sterilized prior to use to prevent contamination, e.g. by ultraviolet light, heating, irradiation or filtration. The differentiation medium may be frozen (e.g. at −20° C. or −80° C.) for storage or transport. The medium may contain one or more antibiotics to prevent contamination. The medium may have an endotoxin content of less that 0.1 endotoxin units per ml, or may have an endotoxin content less than 0.05 endotoxin units per ml. Methods for determining the endotoxin content of culture media are known in the art.

A preferred basal culture medium is a defined synthetic medium that is buffered at a pH of 7.4 (preferably with a pH 7. 2-7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

In some embodiments, a differentiation medium is provided with a basal medium ready for use in a differentiation method. In other embodiments, the differentiation medium is provided without a basal medium, e.g. as a culture medium supplement, and the basal medium (or other components) can be added prior to use in a differentiation method. Accordingly, there is provided any differentiation medium described herein wherein the basal medium is absent or wherein the basal medium is only an optional component.

### Additional Factors

A number of factors have been previously shown to enhance the differentiation of progenitor cells in some contexts. In some embodiments, one or more of these factors are included in the differentiation medium of the invention. These factors include but are not limited to p38 inhibitors, TGF-beta inhibitors, gastrin, glucocorticoids, receptor tyrosine kinase ligands, BMP pathway activators, Hedgehog activators, Hedgehog inhibitors, modulators of mTOR signalling GSK-3 inhibitors, CHIR99021 agonists, AP-1 stimulants, muscarinic acetylcholine receptor agonists, carbachol agonists and cAMP pathway activators. In some embodiments, these factors are selected from p38 inhibitors, TGF-beta inhibitors, gastrin, glucocorticoids, receptor tyrosine kinase ligands, BMP pathway activators, BMP pathway inhibitors, Hedgehog activators, Hedgehog inhibitors, modulators of mTOR signalling GSK-3 inhibitors, CHIR99021 agonists, AP-1 stimulants, muscarinic acetylcholine receptor agonists, carbachol agonists and cAMP pathway activators. In some embodiments, these factors are selected from gastrin, glucocorticoids, receptor tyrosine kinase ligands, BMP pathway activators, BMP pathway inhibitors, Hedgehog activators, Hedgehog inhibitors, modulators of mTOR signalling GSK-3 inhibitors, CHIR99021 agonists, AP-1 stimulants, muscarinic acetylcholine receptor agonists, carbachol agonists and cAMP pathway activators.

### P38 Inhibitor

In some embodiments of the invention, the differentiation medium further comprises a p38 inhibitor, meaning any inhibitor that, directly or indirectly, negatively regulates p38 signalling. In some embodiments, an inhibitor according to the invention binds to and reduces the activity of p38 (GI number 1432). p38 protein kinases are part of the family of mitogen-activated protein kinases (MAPKs). MAPKs are serine/threonine-specific protein kinases that respond to extracellular stimuli, such as environmental stress and inflammatory cytokines, and regulate various cellular activities, such as gene expression, mitosis, differentiation, proliferation, and cell survival/apoptosis. The p38 MAPKs exist as α, β, β2, γ and δ isoforms. A p38 inhibitor is an agent that binds to and reduces the activity of at least one p38 isoform. Various methods for determining if a substance is a p38 inhibitor are known, and might be used in conjunction with the invention. Examples include: phospho-specific antibody detection of phosphorylation at Thr180/Tyr182, which provides a well-established measure of cellular p38 activation or inhibition; biochemical recombinant kinase assays; tumor necrosis factor alpha (TNFα) secretion assays; and DiscoverRx high throughput screening platform for p38 inhibitors (see http.//www.discoverx.com/kinases/literature/biochemical/collaterals/DRx_poster_p38%20KBA.pdf). Several p38 activity assay kits also exist (e.g. Millipore, Sigma-Aldrich).

Various p38 inhibitors are known in the art. In some embodiments, the inhibitor that directly or indirectly negatively regulates p38 signalling is selected from the group consisting of SB-202190, SB-203580, VX-702, VX-745, PD-169316, RO-4402257 and BIRB-796.

In one embodiment, the p38 inhibitor according to the invention binds to and reduces the activity of its target by more than 10%; more than 30%; more than 60%; more than 80%; more than 90%; more than 95%; or more than 99% compared to a control, as assessed by a cellular assay. Examples of cellular assays for measuring target inhibition are well known in the art as described above.

SB-203580 may be added to the differentiation medium at a concentration of between 50 nM and 100 μM, or between 100 nM and 50 μM, or between 1 μM and 50 μM. For example, SB-203580 may be added to the culture medium at approximately 30 μM.

### TGF-Beta Inhibitor

In some embodiments, the differentiation medium further comprises a TGF-beta inhibitor.

TGF-beta signalling is involved in many cellular functions, including cell growth, cell fate and apoptosis. Signalling typically begins with binding of a TGF-beta superfamily ligand to a type II receptor which recruits and phosphorylates a type I receptor. The type I receptor then phosphorylates SMADs, which act as transcription factors in the nucleus and regulate target gene expression.

The TGF-beta inhibitor signalling pathway has previously been implicated in promoting the differentiation of progenitor cells. For example, the addition of TGF-beta to liver explants facilitates the biliary differentiation in vitro (Clotman et al. (2005) Genes Dev. 19(16):1849-54). In addition, it has previously been shown that inclusion of a TGF-beta inhibitor in a differentiation medium can inhibit biliary duct cell-fate and trigger the differentiation of the cells towards a more hepatocytic phenotype (see WO 2012/168930). In particular, inclusion of a TGF-beta inhibitor (such as A83-01) in a differentiation medium was found to enhance the expression of mature hepatocyte markers and increase the number of hepatocyte-like cells.

The TGF-beta superfamily ligands comprise bone morphogenic proteins (BMPs), growth and differentiation factors (GDFs), anti-mullerian hormone (AMH), activin, nodal and TGF-betas. In general, Smad2 and Smad3 are phosphorylated by the ALK4, 5 and 7 receptors in the TGF-beta/activin pathway. By contrast, Smad1, Smad5 and Smad8 are phosphorylated as part of the bone morphogenetic protein (BMP) pathway. Although there is some cross-over between pathways, in the context of this invention, a “TGF-beta inhibitor” or an “inhibitor of TGF-beta signalling” is preferably an inhibitor of the TGF-beta pathway which acts via Smad2 and Smad3 and/or via ALK4, ALK5 or ALK7. Therefore, in some embodiments the TGF-beta inhibitor is not a BMP inhibitor, i.e. the TGF-beta inhibitor is not Noggin. In some embodiments, a BMP inhibitor is added to the culture medium in addition to the TGF-beta inhibitor. Thus the TGF-beta inhibitor may be any agent that reduces the activity of the TGF-beta signalling pathway, preferably the signalling pathway that acts via Smad2 and/or Smad3, more preferably the signalling pathway that acts via ALK4, ALK5 or ALK7.

There are many ways of disrupting the TGF-beta signalling pathway that are known in the art and that can be used in conjunction with this invention. For example, the TGF-beta signalling may be disrupted by: inhibition of TGF-beta expression by a small-interfering RNA strategy; inhibition of furin (a TGF-beta activating protease); inhibition of the pathway by physiological inhibitors; neutralisation of TGF-beta with a monoclonal antibody; inhibition with small-molecule inhibitors of TGF-beta receptor kinase 1 (also known as activin receptor-like kinase, ALK5), ALK4, ALK6, ALK7 or other TGF-beta-related receptor kinases; inhibition of Smad 2 and Smad 3 signalling e.g. by overexpression of their physiological inhibitor, Smad 7, or by using thioredoxin as an Smad anchor disabling Smad from activation (Fuchs, O. Inhibition of TGF—Signalling for the Treatment of Tumor Metastasis and Fibrotic Diseases. Current Signal Transduction Therapy, Volume 6, Number 1, January 2011, pp. 29-43(15)).

Various methods for determining if a substance is a TGF-beta inhibitor are known and might be used in conjunction with the invention. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al. (2005) Br J Pharmacol. 145(2): 166-177). New TGF-beta inhibitors can therefore be easily identified by the skilled person in the art.

A TGF-beta inhibitor according to the present invention may be a protein, peptide, small-molecules, small-interfering RNA, antisense oligonucleotide, aptamer or antibody. The inhibitor may be naturally occurring or synthetic. In one embodiment, the TGF-beta inhibitor is an inhibitor of ALK4, ALK5 and/or ALK7. For example, the TGF-beta inhibitor may bind to and directly inhibit ALK4, ALK5 and/or ALK7. Examples of preferred small-molecule TGF-beta inhibitors that can be used in the context of this invention include but are not limited to the small molecule inhibitors listed in table 2 below.

TABLE 2
Small-molecule TGF-beta inhibitors targeting receptor kinases

| | | IC50 | Mol | | |
|---|---|---|---|---|---|
| Inhibitor | Targets | (nM) | Wt | Name | Formula |
| A83-01 | ALK5 | 12 | 421.52 | 3-(6-Methyl-2-pyridinyl)-N- | C25H19N5S |
| | (TGF-βR1) | | | phenyl-4-(4-quinolinyl)-1H- | |
| | ALK4 | 45 | | pyrazole-1-carbothioamide | |
| | ALK7 | 7.5 | | | |
| SB-431542 | ALK5 | 94 | 384.39 | 4-[4-(1,3-benzodioxol-5-yl)-5-(2- | C22H16N4O3 |
| | ALK4 | | | pyridinyl)-1H-imidazol-2- | |
| | ALK7 | | | yl]benzamide | |
| SB-505124 | ALK5 | 47 | 335.4 | 2-(5-benzo[1,3]dioxol-5-yl-2- | C20H21N3O2 |
| | ALK4 | 129 | | tert-butyl-3Himidazol- | |
| | | | | 4-yl)-6-methylpyridine | |
| | | | | hydrochloride hydrate | |
| SB-525334 | ALK5 | 14.3 | 343.42 | 6-[2-(1,1-Dimethylethyl)-5-(6- | C21H21N5 |
| | | | | methyl-2-pyridinyl)-1H- | |
| | | | | imidazol-4-yl]quinoxaline | |
| SD-208 | ALK5 | 49 | 352.75 | 2-(5-Chloro-2-fluorophenyl)-4- | C17H10ClFN6 |
| | | | | [(4-pyridyl)amino]pteridine | |
| LY-36494 | TGR-βRI | 59 | 272.31 | 4-[3-(2-Pyridinyl)-1H-pyrazol-4- | C17H12N4 |
| | TGR-βRII | 400 | | yl]-quinoline | |
| | MLK-7K | 1400 | | | |
| SJN-2511 | ALK5 | 23 | 287.32 | 2-(3-(6-Methylpyridine-2-yl)- | C17H13N5 |
| | | | | 1H-pyrazol-4-yl)-1,5- | |
| | | | | naphthyridine | |

In some embodiments, the TGF-beta inhibitor is a small molecule inhibitor optionally selected from the group consisting of: A83-01, SB-431542, SB-505124, SB-525334, LY 364947, SD-208 and SJN 2511.

In some embodiments, no more than one TGF beta inhibitor is present in the differentiation medium. In other embodiments, more than one TGF beta inhibitor is present in the differentiation medium, e.g. 2, 3, 4 or more. In some embodiments, a differentiation medium of the invention comprises one or more of any of the inhibitors listed in table 2. A differentiation medium may comprise any combination of one inhibitor with another inhibitor listed. For example, a medium may comprise SB-525334 or SD-208 or A83-01; or SD-208 and A83-01. The skilled person will appreciate that a number of other small-molecule inhibitors exist that are primarily designed to target other kinases, but at high concentrations may also inhibit TGF-beta receptor kinases. For example, SB-203580 is a p38 MAP kinase inhibitor that, at high concentrations (for example, approximate 10 μM or more) is thought to inhibit ALK5. Any such inhibitor that inhibits the TGF-beta signalling pathway can also be used in the context of this invention.

In some embodiments, the TGF-beta inhibitor (e.g. A83-01) is present in the differentiation medium at at least 1 nM, for example, at least 5 nM, at least 50 nM, at least 100 nM, at least 300 nM, at least 450 nM or at least 475 nM. For example, the TGF-beta inhibitor (e.g. A83-01) is present in the differentiation medium at 1 nM-200 μM, 10 nM-200 μM, 100 nM-200 μM, 1 μM-200 μM, 10 nM-100 μM, 50 nM-100 μM, 50 nM-10 μM, 100 nM-1 μM, 200 nM-800 nM, 350-650 nM or at about 500 nM. Accordingly, in some embodiments, the differentiation medium comprises A83-01 at a concentration of about 500 nM.

In some embodiments, the differentiation medium does not comprise a TGF-beta inhibitor. In some embodiments, the differentiation medium does not comprise A83-01.

### Gastrin

In some embodiments, the differentiation medium of the invention further comprises gastrin. In some embodiments, the differentiation medium of the invention comprises gastrin at a concentration of 0.01-500 nM, 0.1-100 nM, 1-100 nM, 1-20 nM or 5-15 nM. For example, in some embodiments, the differentiation medium of the invention comprises gastrin at a concentration of about 10 nM.

### Glucocorticoid

In some embodiments, the differentiation medium further comprises a glucocorticoid.

Glucocorticoids are a class of corticosteroids, which are a class of steroid hormones. Glucocorticoids are corticosteroids that bind to the glucocorticoid receptor. Cortisol is the most important human glucocorticoid. Hydrocortisone is the synthetic version of cortisol. Many other synthetic glucocorticoids with related structures exist (e.g. prednisone, prednisolone, methylprednisolone, dexamethasone, betamethasone, triamcinolone, beclomethasone and fludrocortisone acetate). Glucocorticoids have varying potency for activating a glucocorticoid receptor. This is called the glucocorticoid potency and is usually measured in comparison to cortisol. The biochemistry, pharmacology and mechanism of action of various glucocorticoids are reviewed in, for example, Cecil Textbook of Medicine (1988), pages 128-130, and The Science and Practice of Pharmacy 20th Edition (2000), pages 1363-1370.

In some embodiments, the differentiation medium comprises a glucocorticoid. Any suitable glucocorticoid may be used. In some embodiments, the glucocorticoid is selected from one or more of the following: cortisol, cortisone, hydrocortisone acetate, hydrocortisone hydrochloride, hydrocortisone valerate, prednisone, prednisolone, methylprednisolone, dexamethasone, betamethasone, betamethasone dipropionate, betamethasone valerate, triamcinolone, triamcinolone acetonide, beclomethasone, beclomethasone dipropionate, fludrocortisone, fludrocortisone acetate, fluticasone, fluticasone acetonide, fluticasone propionate, flunisolide, budesonide, clobetasol, clobetasol propionate, difluorasone, difluorasone diacetate, halobetasol, halobetasol propionate, amcinonide, desoximetasone, fluocinonide, fluocinonide acetonide, halcinonide, mometasone, mometasone furoate, fluandrenolide, prednicarbate, aclometasone, aclometasone dipropionate, desonide, flucinolone, flucinolone acetonide, pramoxine and pramoxine hydrochloride.

Dexamethasone is one of the most potent glucocorticoids and is a preferred glucocorticoid for use in the differentiation medium of the invention. In some embodiments, the glucocorticoid is any glucocorticoid with the same or higher glucocorticoid potency than dexamethasone.

Betamethasone and fludrocortisone acetate are also highly potent glucocorticoids. Accordingly, betamethasone is a preferred glucocorticoid for use in the differentiation medium of the invention.

In some embodiments, the glucocorticoid is any glucocorticoid with the same or higher glucocorticoid potency than betamethasone. Accordingly, fludrocortisone acetate is a preferred glucocorticoid for use in the differentiation medium of the invention. In some embodiments, the glucocorticoid is any glucocorticoid with the same or higher glucocorticoid potency than fludrocortisone acetate.

In some embodiments, the glucocorticoid is any glucocorticoid with the same or higher glucocorticoid potency than cortisol.

A list of exemplary glucocorticoids for use in the differentiation medium of the invention is provided below. The potency presented refers to oral dosing.

| Glucocorticoid | Glucocorticoid potency |
|---|---|
| Cortisol (hydrocortisone) | 1 |
| Cortisone | 0.8 |
| Prednisone | 3.5-5 |
| Prednisolone | 4 |
| Methylprednisolone | 5-7.5 |
| Dexamethasone | 25-80 |
| Betamethasone | 25-30 |
| Triamcinolone | 5 |
| Fludrocortisone acetate | 15 |

In some embodiments, the glucocorticoid (e.g. dexamethasone) is used at a concentration of 0.01-150 μM, 0.1-15 μM, 0.5-10 μM or 1-5 μM. In a preferred embodiment, the glucocorticoid is dexamethasone. For example, in some embodiments, the differentiation medium comprises dexamethasone at a concentration of about 3 μM.

In some embodiments, the differentiation medium does not comprise a glucocorticoid.

### Receptor Tyrosine Kinase Ligands

In some embodiments, a differentiation medium of the invention further comprises one or more receptor tyrosine kinase ligand.

Receptor tyrosine kinases (RTKs) are high-affinity cell surface receptors for polypeptide growth factors, cytokines, and hormones. RTKs are key regulators of cell maintenance, growth and development, and also to have a critical role in the development and progression of many types of cancer. In the context of the invention, a receptor tyrosine kinase ligand is any ligand that activates an RTK. Many receptor tyrosine kinase ligands are mitogenic growth factors. Thus in some embodiments, the one or more receptor tyrosine kinase ligands in the differentiation medium comprises one or more mitogenic growth factor.

There are approximately 20 different known classes of RTKs, including RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family), RTK class XVII (MuSK receptor family). In some embodiments, the one or more receptor tyrosine kinase ligands comprises ligands for one or more, or all of these 20 classes of RTKs.

In some embodiments, the one or more receptor tyrosine kinase ligands comprises a ligand for RTK class IV (FGF receptor family). In some embodiments, the one or more receptor tyrosine kinase ligands comprises a ligand for RTK class VI (HGF receptor family). In some embodiments, the one or more receptor tyrosine kinase ligands comprises a ligand for RTK class IV (FGF receptor family) and a ligand for RTK class VI (HGF receptor family).

Thus, in some embodiments, the one or more receptor tyrosine kinase ligands in the differentiation medium are selected from the group consisting of fibroblast growth factor (FGF) and hepatocyte growth factor (HGF). In some embodiments, the one or more receptor tyrosine kinase ligands comprises FGF and HGF. In some embodiments, only one receptor tyrosine kinase ligand is included in the differentiation medium, for example, wherein the receptor tyrosine kinase is selected from FGF and HGF.

The FGF is preferably an FGF able to bind to FGFR2 or FGFR4 and is preferably FGF19.

Three or more, for example, 3, 4, 5 or more receptor tyrosine kinase ligands may be used in the differentiation media of the invention.

As explained above, the inventors found that inhibition of the EGFR pathway contributed to enteroendocrine cell differentiation. Therefore, preferably, the one or more receptor tyrosine kinase ligands does not comprise a ligand that activates EGFR (e.g. EGF). In some embodiments, the differentiation medium comprises less than 1 mM EGF.

### BMP Pathway Activator

The inventors have surprisingly found that including an BMP pathway activator in an EEC differentiation medium increases the presence of secretin-secreting cells among the differentiated EEC population (see FIGS. 13 and 14). Therefore, in some embodiments, the differentiation medium further comprises a BMP pathway activator. In some embodiments, the differentiation medium does not comprise a BMP pathway inhibitor (e.g. Noggin). In some embodiments, the differentiation medium further comprises a BMP pathway activator and does not comprise a BMP pathway inhibitor (e.g. Noggin).

Methods for identifying suitable BMP activators are known in the art. A suitable assay for measuring BMP activity is described in Zilberberg et al., BMC Cell Biology 2007 8:41.

In some embodiments, the BMP pathway activator is selected from BMP7, BMP4 and BMP2. BMP7 is preferred. BMP7 induces the phosphorylation of SMAD1 and SMAD5. Thus in some embodiments, the BMP pathway activator is any compound that is capable of inducing the phosphorylation of SMAD1 and SMAD5. In addition, where BMP7 is mentioned, any compound that induces the phosphorylation of SMAD1 or SMAD5 can be used instead of BMP7.

In some embodiments, the BMP pathway activator, such as BMP4 or BMP7 is present in the differentiation medium at at least 0.01 ng·ml, at least 0.1 ng/ml, at least 1 ng/ml, at least 10 ng/ml, at least 20 ng/ml, at least 25 ng/ml, at least 100 ng/ml, at least 500 ng/ml, at least 1 g/ml, at least g/ml or at least 50 g/ml. In some embodiments, the BMP pathway activator, such as BMP4 or BMP7 is present in the differentiation medium from about 0.01 ng/ml to about 500 ng/ml, from about 1 ng/ml to about 500 ng/ml, from about 10 ng/ml to about 500 ng/ml, from about 20 ng/ml to about 500 ng/ml. In some embodiments, the BMP pathway activator, such as BMP4 or BMP7, is present in the differentiation medium from about 0.01 ng/ml to about 200 ng/ml, from about 0.1 ng/ml to about 100 ng/ml, from about 1 ng/ml to about 100 ng/ml, from about 10 ng/ml to about 100 ng/ml, from about 10 ng/ml to about 50 ng/ml, from about 15 ng/ml to about 30 ng/ml. In some embodiments, the BMP pathway activator, such as BMP4 or BMP7 is present in the differentiation medium at about 25 ng/ml. In some embodiments, BMP4 is present in the differentiation medium from about 0.1 g/ml to about 50 g/ml, from about 1 to about 50 g/ml, from about 5 g/ml to about 25 g/ml or from about 5 g/ml to about 15 g/ml. In some embodiments, BMP4 is present in the differentiation medium at about 10 g/ml.

In some embodiments, the differentiation medium does not comprise a BMP pathway activator.

There is also provided a method for increasing secretin secretion in a population of cells comprising EECs, wherein the method comprises contacting the population of cells with a BMP activator.

There is also provided a method for decreasing GLP-1 secretion in a population of cells comprising EECs, wherein the method comprises contacting the population of cells with a BMP activator.

There is also provided a method for increasing expression of Pyy and/or Nts in a population of cells comprising EECs, wherein the method comprises contacting the population of cells with a BMP activator.

### BMP Inhibitor

The inventors have also surprisingly found that including an inhibitor of BMP signalling in an EEC differentiation medium promotes a crypt hormone signature in the differentiated EEC population (see Example 5). A crypt hormone signature is characterized by high expression of GLP-1 and by lack of expression of secretin. Therefore, in some embodiments, the differentiation medium of the invention further comprises a BMP inhibitor. In these embodiments, the differentiation medium preferably does not comprise a BMP activator.

BMPs are small signalling molecules that bind to two classes of cell surface bone morphogenetic protein receptors (BMPR-I and BMPRII). The BMPR-I receptor class consists of three receptor types, activin receptor-like kinase-2 (ALK-2 or ActR-IA), ALK-3 (BMPR-IA) and ALK-6 (BMPR-IB). The BMPR-II receptor class is comprised of three receptor types, BMPR-II, ActR-IIA and ActR-IIB. Binding of BMPs results in the formation of heterotetrameric complexes containing two type I and two type II receptors. In addition to an extracellular binding domain, each BMP receptor contains an intracellular serine/threonine kinase domain. Following binding of BMPs, constitutively active type II receptor kinases phosphorylate type I receptor kinase domains that in turn phosphorylate BMP-responsive SMADs 1, 5, and 8, which can enter the cell nucleus and function as transcription factors. Phosphorylation of these specific SMADs results in various cellular effects, including growth regulation and differentiation. A BMP inhibitor is any inhibitor that results in a significant reduction in signaling via these pathways. For example, a BMP inhibitor may be able to disrupt the interaction of a BMP with a BMP receptor; bind to a BMP receptor and inhibit activation of downstream signalling; inhibit phosphorylation of Smad 1, Smad 5 or Smad 8; inhibit translocation of Smad 1, Smad 5 or Smad 8 to the nucleus; inhibit SMAD 1, SMAD 5 or SMAD 8 mediated transcription of target genes; or inhibit expression, folding or secretion of a BMP. In some embodiments, the BMP inhibitor reduces signaling via the BMPR-I receptor class. In some embodiments, the BMP inhibitor reduces signaling via BMPR-II receptor class. In some embodiments, the BMP inhibitor reduces signaling via SMAD 1/5/8. The inhibition may be direct or indirect.

Many BMP inhibitors are known in the art, e.g. as disclosed in Cuny, et al., (2008) Structure-activity relationship study of bone morphogenetic protein (BMP) signaling inhibitors. Bioorg Med Chem Lett 18: 4388-4392. Any of these BMP inhibitors are suitable for use in the methods of the invention. Methods for identifying suitable BMP inhibitors are known in the art. A suitable assay is described in Zilberberg et al., BMC Cell Biology 2007 8:41. Another suitable assay for a BMP inhibitor (in particular a BMP inhibitor that inhibits phosphorylation of Smad 1, 5 or 8 via ALK2 and ALK3) can be identified by a person skilled in the art using the cytobot cellular ELISA assay described in Cuny, et al., (2008) Structure-activity relationship study of bone morphogenetic protein (BMP) signaling inhibitors. Bioorg Med Chem Lett 18: 4388-4392.

In some embodiments the BMP inhibitor is selected from noggin, chordin, follistatin, gremlin, tsg (twisted gastrulation), sog (short gastrulation), dorsomorphin and LDN193189. In some embodiments, the BMP inhibitor is selected from:
a. dorsomorphin or LDN193189 or an analog or variant thereof, and/or
b. noggin, sclerostin, chordin, CTGF, follistatin, gremlin, tsg, sog or an analog or variant thereof.

In some preferred embodiments the BMP inhibitor is noggin. Noggin is particularly suitable for in vitro culture methods. In other preferred embodiments, the BMP inhibitor is LDN193189. LDN193189 is particularly suitable for in vivo methods of treatment for because it is orally available and so suitable for oral administration (see further comments on methods of treatment later).

In some embodiments, noggin is included in the differentiation medium at a final concentration of between 1 and 1000 ng/ml, between 10 and 1000 ng/ml, between 100 and 1000 ng/ml, between 1 and 500 ng/ml, between 1 and 200 ng/ml, between 1 and 100 ng/ml, between 10 and 500 ng/ml, between 20 and 500 ng/ml, between 10 and 200 ng/ml, between 20 and 200 ng/ml, between 50 and 500 ng/ml, or between 50 and 200 ng/ml. In some embodiments, noggin is included in the differentiation medium at a final concentration of about 100 ng/ml.

In some embodiments, LDN193189 is included in the differentiation medium at a final concentration of between 1 nM and 10 μM, between 5 nM and 10 μM, between 10 nM and 10 μM, between 100 nM and 10 μM, between 1 μM and 10 μM, or between 1 μM and 5 μM. In some embodiments, LDN193189 is included in the differentiation medium at a final concentration of at least 1 nM, at least 5 nM, at least 10 nM, at least 100 nM, at least 1 μM, or about 10 μM.

There is also provided a method for increasing GLP-1 secretion in a population of cells comprising EECs, wherein the method comprises contacting the population of cells with a BMP inhibitor. There is also provided a method for decreasing secretin secretion in a population of cells comprising EECs, wherein the method comprises contacting the population of cells with a BMP inhibitor.

There is also provided a method for increasing Tac1 expression in a population of cells comprising EECs, wherein the method comprises contacting the population of cells with a BMP inhibitor.

There is also provided a method for increasing Gcg expression in a population of cells comprising EECs, wherein the method comprises contacting the population of cells with a BMP inhibitor.

### Hedgehog Activators and Inhibitors

The inventors have surprisingly found that including an inhibitor of Hedgehog signalling in an EEC differentiation medium can in some contexts increase the presence of GLP1- and CCK-secreting cells among the differentiated EEC population (see FIG. 14). Therefore, in some embodiments, the differentiation medium of the invention further comprises one or more Hedgehog inhibitors. The one or more Hedgehog inhibitors may be any suitable inhibitor that decreases Hedgehog signalling in a cell. This medium is suitable for obtaining an EEC population that is enriched in GLP1- and CCK-secreting cells. Alternatively, an EEC population in which the number of GLP1- and CCK-secreting cells has been depleted is obtainable using a differentiation medium of the invention that further comprises a Hedgehog activator. Accordingly, in some embodiments, the differentiation medium of the invention further comprises one or more Hedgehog activators. The one or more Hedgehog activators may be any suitable activator that increases Hedgehog signalling in a cell.

Mammalian hedgehog (Hh) ligands include Sonic hedgehog (Shh), Indian hedgehog (Ihh) and Desert hedgehog (Dhh). Hh ligands are typically synthesized as precursor proteins that undergo autocatalytic cleavage and concomitant cholesterol modification at the carboxy terminus and palmitoylation at the amino terminus, resulting in a secreted, dually-lipidated protein. Hh ligands are released from the cell surface through the combined actions of Dispatched and Scube2, and subsequently trafficked over multiple cells through interactions with the cell surface proteins LRP2 and the Glypican family of heparan sulfate proteoglycans (GPC1-6).

Hh proteins initiate signalling through binding to the canonical receptor Patched (PTCH1) and to the co-receptors GAS1, CDON and BOC. Hh binding to PTCH1 results in derepression of the GPCR-like protein Smoothened (SMO) that results in SMO accumulation in cilia and phosphorylation of its cytoplasmic tail. SMO mediates downstream signal transduction that includes dissociation of GLI proteins (the transcriptional effectors of the Hh pathway) from kinesin-family protein, Kif7, and the key intracellular Hh pathway regulator SUFU.

In some embodiments, the one or more Hedgehog inhibitors comprise a SMO inhibitor. In some embodiments, the SMO inhibitor is cyclopamine or a cyclopamine-competitive inhibitor (e.g. vismogenib, saridegib or cyclopamine). In other embodiments, the SMO inhibitor is not a cyclopamine-competitive inhibitor (e.g. itraconazole).

In some embodiments, the one or more Hedgehog inhibitors comprise an anti-PTCH1 antibody that inhibits Hh binding to PTCH1 (see, e.g., Nakamura et al. (2007) Anticancer Research 27:3743-3748).

In some embodiments, the one or more Hedgehog activators comprise an SMO activator. In some embodiments, the SMO activator is SAG (Hh-Ag1.3) or purmorphamine.

Hedgehog inhibitors and Hedgehog activators can be identified using methods known in the art, for example, using a RT-PCR method to determine the mRNA levels of Gli1 and Ptch1 (see, e.g., Nakamura et al. (2007) Anticancer Research 27:3743-3748). Gli1 and Ptch1 are target genes of Gli1 trans-activation, and so can be used as markers of Hh signalling pathway activity. For example, suppressed expression of Gli1 and Ptch1 is indicative of decreased Hh signalling pathway activity.

In one embodiment, the Hedgehog inhibitor according to the invention reduces the activity of the Hedgehog signalling pathway by more than 10%; more than 30%; more than 60%; more than 80%; more than 90%; more than 95%; or more than 99% compared to a control, as assessed by a RT-PCR assay. Examples of RT-PCR assays for measuring inhibition are well known in the art as described above.

In one embodiment, the Hedgehog activator according to the invention increases the activity of the Hedgehog signalling pathway by more than 10%; more than 30%; more than 60%; more than 80%; more than 90%; more than 95%; or more than 99% compared to a control, as assessed by a RT-PCR assay. Examples of RT-PCR assays for measuring activation are well known in the art as described above.

In some embodiments, the Hedgehog inhibitor is used at a concentration of 0.01-200 μM, 0.01-100 μM, 0.05-100 μM, 0.1-50 μM, 0.1-20 μM, 1-100 μM, 1-50 μM, 1-30 μM, 1-10 μM, 5-100 μM, 5-50 μM or 5-20 μM. In some embodiments, the Hedgehog inhibitor (e.g. vismogenib) is present at a concentration of about 5 μM.

In some embodiments, the Hedgehog activator is used at a concentration of 0.01-200 μM, 0.01-100 μM, 0.05-100 μM, 0.1-50 μM, 0.1-20 μM, 1-100 μM, 1-50 μM, 1-30 μM, 1-10 μM, 5-100 μM, 5-50 μM or 5-20 μM. In some embodiments, the Hedgehog activator (e.g. SAG (Hh-Ag1.3) or purmorphamine) is present at a concentration of about 5 μM.

In some embodiments, the differentiation medium of the invention comprises two, three, four or more Hedgehog inhibitors.

In some embodiments, the differentiation medium of the invention comprises two, three, four or more Hedgehog activators.

In some embodiments, the differentiation medium does not comprise a Hedgehog inhibitor.

Modulators of mTOR Signalling

The inventors have surprisingly found that including a modulator of mTOR signalling may affect the relative ratios of the EEC subtypes (e.g. mTOR activators may promote differentiation towards GLU-expressing EECs). In some embodiments, the differentiation medium of the invention further comprises one or more modulators of mTOR signalling. In some embodiments, the one or more modulators of mTOR signalling comprise an inhibitor of mTOR signalling. The one or more mTOR inhibitors may be any suitable inhibitor that decreases mTOR signalling in a cell. In some embodiments, the one or more modulators of mTOR signalling comprise an activator of mTOR signalling (e.g. MHY1485). The one or more mTOR activators may be any suitable activator that increases mTOR signalling in a cell.

The mechanistic target of rapamycin (mTOR) is an atypical serine/threonine kinase that is present in two distinct complexes. The first, mTOR complex 1 (mTORC1), is composed of mTOR, Raptor, GβL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis, or limit catabolic processes such as autophagy. The small GTPase Rheb, in its GTP-bound state, is a necessary and potent stimulator of mTORC1 kinase activity, which is negatively regulated by its GAP, the tuberous sclerosis heterodimer TSC1/2. Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, notably the v-ATPase, Ragulator, the Rag GTPases, and GATOR1/2. The second complex, mTOR complex 2 (mTORC2), is composed of mTOR, Rictor, GβL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation.

In some embodiments, the activator of mTOR signalling is MHY1485.

In some embodiments, the inhibitor of mTOR signalling is rapamycin or a rapamycin analogue (e.g. rapamycin, deforolimus (AP23573), everolimus (RAD001), and temsirolimus (CCI-779)). In some embodiments, the inhibitor of mTOR signalling is an ATP-competitive mTOR kinase inhibitor (e.g. MLN0128, pp242 or AZD8055).

mTOR inhibitors and mTOR activators can be identified using methods known in the art, for example, using an ELISA-based mTOR kinase assay (e.g. using a K-LISA™ mTOR activity kit, which is an ELISA-based activity assay for detection of phosphorylation of a p70S6K-GST fusion protein (a specific mTOR substrate) in the presence of ATP).

In one embodiment, the mTOR inhibitor according to the invention reduces the activity of the mTOR signalling pathway by more than 10%; more than 30%; more than 60%; more than 80%; more than 90%; more than 95%; or more than 99% compared to a control, as assessed by an ELISA-based assay. Examples of ELISA-based assays for measuring inhibition are well known in the art as described above.

In one embodiment, the mTOR activator according to the invention increases the activity of the mTOR signalling pathway by more than 10%; more than 30%; more than 60%; more than 80%; more than 90%; more than 95%; or more than 99% compared to a control, as assessed by an ELISA-based assay. Examples of ELISA-based assays for measuring activation are well known in the art as described above.

In some embodiments, the mTOR inhibitor is used at a concentration of 0.01-200 μM, 0.01-100 μM, 0.05-100 μM, 0.1-50 μM, 0.1-20 μM, 1-100 μM, 1-50 μM, 1-30 μM, 1-10 μM, 5-100 μM, 5-50 μM or 5-20 μM. In some embodiments, the mTOR inhibitor (e.g. rapamycin, deforolimus (AP23573), everolimus (RAD001) or temsirolimus (CCI-779)) is present at a concentration of about 5 μM.

In some embodiments, the mTOR activator is used at a concentration of 0.01-200 μM, 0.01-100 μM, 0.05-100 μM, 0.1-50 μM, 0.1-20 μM, 1-100 μM, 1-50 μM, 1-30 μM, 1-10 μM, 5-100 μM, 5-50 μM or 5-20 μM. In some embodiments, the mTOR activator (e.g. MHY1485) is present at a concentration of about 5 μM.

In some embodiments, the differentiation medium of the invention comprises two, three, four or more mTOR inhibitors.

In some embodiments, the differentiation medium of the invention comprises two, three, four or more mTOR activators.

### GSK-3 Inhibitor

It was previously found that the inclusion of a GSK-3 inhibitor (e.g. CHIR99021) also improved the differentiation of epithelial stem cells (see GB 1603569.3). As explained above, GSK-3 is a component of the β-catenin destruction complex. Inhibiting GSK-3 activity results in reduced destruction of β-catenin, and so GSK-3 inhibitors are Wnt agonists. The enhanced differentiation elicited by the inclusion of a GSK-3 inhibitor in the differentiation medium was greatly increased by the addition of a Wnt inhibitor (iCRT3) that inhibits TCF/LEF-mediated transcription downstream from the destruction complex. For example, there was a pronounced improvement in the expression of hepatocyte markers (albumin and Cyp3A4) when both a Wnt agonist (CHIR99021) and a Wnt inhibitor (iCRT3) are included in a differentiation medium for liver progenitor cells. Without wishing to be bound by any theory, the inventors believe that GSK-3 inhibitors could both stimulate and inhibit differentiation in epithelial stem cells (with the stimulation being stronger than the inhibition). The inhibition of differentiation by GSK-3 inhibitors is thought to occur via the promotion of β-catenin destruction. In contrast, the promotion of differentiation by GSK-3 inhibitors is thought to occur by a distinct effector mechanism. Accordingly, when a GSK-3 inhibitor is included in the differentiation medium, the Wnt inhibitor should be a Wnt inhibitor that inhibits TCF/LEF-mediated transcription downstream from the destruction complex in the GSK-3 inhibitor-comprising differentiation medium counteracts the differentiation-inhibiting effects of the GSK-3 inhibitor. Wnt inhibitors that act downstream of the destruction complex include Wnt inhibitors in class (7) mentioned above, i.e. inhibitors of β-catenin target gene expression, including inhibitors of the β-catenin:TCF/Lef transcription complex, such as inhibitors that disrupt the β-catenin:TCF-4 complex (e.g. iCRT3, CGP049090, PKF118310, PKF115-584, ZTM000990, PNU-74654, BC21, iCRT5, iCRT14 or FH535) and inhibitors of the histone deacetylase SIRT1 (e.g. cambinol).

Thus, the differentiation-promoting effects of GSK-3 inhibitors remain unaffected and are added to by the differentiation-promoting effects of the Wnt inhibitor.

Accordingly, in some embodiments, the differentiation medium of the invention comprises a GSK-3 inhibitor. Any suitable GSK-3 inhibitor may be used. A GSK-3 inhibitor is defined as being an agent that reduces GSK-3 kinase activity.

Two different isoforms of GSK-3 are found in humans (GSK-3a and GSK-30). Inhibitors are known that inhibit one or both of these isoforms. Accordingly, in some embodiments, the GSK-3 inhibitor inhibits GSK-3a and GSK-30. In some embodiments, the GSK-3 inhibitor inhibits GSK-3a but does not inhibit GSK-30. In some embodiments, the GSK-3 inhibitor inhibits GSK-30 but does not inhibit GSK-3a.

In some embodiments, the differentiation medium comprises more than one GSK-3 inhibitor (e.g. two, three, four, five or more GSK-3 inhibitors).

In some embodiments, the GSK-3 inhibitor is CHIR99021.

In some embodiments, the GSK-3 inhibitor is added to the media in an amount effective to inhibit a GSK-3 activity in a cell by at least 10%, more preferred at least 20%, more preferred at least 30%, more preferred at least 50%, more preferred at least 70%, more preferred at least 90%, more preferred 100%, relative to a level of said GSK-3 activity in the absence of said molecule, as assessed in the same cell type. As is known to a skilled person, GSK-3 activity can be measured by monitoring phosphorylation of specific GSK-3 substrates, e.g. β-catenin (see, e.g., Cole et al. (2008) Methods Mol Biol. 468:45-65). New GSK-3 inhibitors can therefore easily be identified by a skilled person using an assay known in the art.

In some embodiments, the GSK-3 inhibitor is selected from one or more of the following: CHIR99201, 6-BIO, Dibromocantharelline, Hymenialdesine, Indirubins, Meridianins, CT98014, CT98023, CT99021, TWS119, SB-216763, SB-41528, AR-A014418, AZD-1080, Alsterpaullone, Cazpaullone, Kenpaullone, Aloisines, Manzamine A, Palinurine, Tricantine, TDZD-8, NP00111, NP031115, Tideglusib, HMK-32 and L803-mts.

In some embodiments, the differentiation medium of the invention comprises a GSK-3 inhibitor at a concentration of 0.001-500 μM, 0.01-150 μM, 0.1-100 μM, 1-100 μM, 0.5-50 μM, 1-50 μM, 1-20 μM or 1-5 μM.

In some embodiments, the differentiation medium of the invention comprises CHIR99021 at a concentration of 0.01-150 μM, 0.1-100 μM, 1-100 μM, 0.5-50 μM, 1-50 μM, 1-20 μM or 1-5 μM.

For example, in some embodiments, the differentiation medium of the invention comprises CHIR99021 at a concentration of about 3 μM.

In some embodiments, the differentiation medium of the invention comprises a GSK-3 inhibitor and a Wnt inhibitor that acts downstream of the destruction complex such as inhibitors of β-catenin target gene expression as described earlier. For example, in some embodiments, differentiation medium of the invention comprises a GSK-3 inhibitor and an inhibitor of β-catenin target gene expression. Inhibitors of β-catenin target gene expression include inhibitors of the β-catenin:TCF/Lef transcription complex, such as inhibitors that disrupt the β-catenin:TCF-4 complex (e.g. iCRT3, CGP049090, PKF118310, PKF115-584, ZTM000990, PNU-74654 or BC21) and inhibitors of the histone deacetylase SIRT1 (e.g. cambinol).

Accordingly, in some embodiments, the differentiation medium of the invention comprises a GSK-3 inhibitor (e.g. at a concentration of 0.001-500 μM, 0.01-150 μM, 0.1-100 μM, 0.5-50 μM, 1-20 μM or 1-5 μM) and an inhibitor of β-catenin target gene expression (e.g. at a concentration of 0.001-500 μM, 0.01-150 μM, 0.1-100 μM, 0.5-50 μM, 1-500 μM, 1-150 μM, 1-100 μM, 1-50 μM, 1-20 μM or 1-5 μM). In some embodiments, the inhibitor of β-catenin target gene expression is an inhibitor of the β-catenin:TCF/Lef transcription complex.

Accordingly, in some embodiments, the differentiation medium of the invention comprises CHIR99021 (e.g. at a concentration of 0.01-150 μM, 0.1-100 μM, 0.5-50 μM, 1-20 μM or 1-5 μM) and iCRT3 (e.g. at a concentration of 0.05-150 μM, 0.1-150 μM, 1-150 μM, 0.5-100 μM, 1-100 μM or 10-80 μM). For example, in some embodiments, the differentiation medium of the invention comprises CHIR99021 at a concentration of about 3 μM and iCRT3 at a concentration of about 50 μM.

In some embodiments, the differentiation medium does not comprise a GSK-3 inhibitor.

### CHIR99021 Agonists

As explained above, it was previously found that inclusion of a Wnt agonist, CHIR99021, in a differentiation medium promotes differentiation. Accordingly, in some embodiments, the differentiation medium of the invention comprises CHIR99021 or a CHIR99021 agonist. A CHIR99021 agonist is defined herein as being an agent that shares one or more biological activities with CHIR99021.

### AP-1 Stimulants

The activating protein 1 (AP-1) complex is a transcription factor that is a heterodimer composed of proteins belonging to the Fos protein family, Jun, ATF and JDP families. This transcription factor regulates gene expression in response to a variety of stimuli including cytokines, growth factors, stress and bacterial and viral infections.

It was previously observed that human liver organoids have a reduced expression of the components of the AP-1 complex in comparison to primary hepatocytes. Based on this observation, they hypothesised that stimulating AP-1 complex formation would promote differentiation of epithelial stem cells in the liver organoids to a hepatocyte fate. The inventors found that including an AP-1 stimulant, carbachol (2-[(Aminocarbonyl)oxy]-N,N,N-trimethylethanaminium chloride), in a differentiation medium increased the expression of hepatocyte markers (including Cyp3A4 and albumin).

The AP-1 stimulant previously tested is a muscarinic acetylcholine receptor agonist. There are five subtypes of muscarinic acetylcholine receptor (M₁-M₅). These are G-protein-coupled receptors that are found in the cell membranes of various cell types (e.g. neurones).

It is envisaged that an AP-1 stimulant or muscarinic acetylcholine receptor agonist may also enhance the differentiation of epithelial stem cells from tissues other than liver (e.g. intestine, stomach, pancreas or lung).

Accordingly, in some embodiments, the differentiation medium of the invention comprises an AP-1 stimulant. In some embodiments, the AP-1 stimulant is a muscarinic acetylcholine receptor agonist. Any suitable muscarinic acetylcholine receptor agonist may be used. In some embodiments, the muscarinic acetylcholine receptor agonist is an M₃ muscarinic acetylcholine receptor agonist (e.g. acetylcholine, bethanechol, carbachol, oxotremorine or pilocarpine).

In some embodiments, the muscarinic acetylcholine receptor agonist is selected from one or more of the following: acetylcholine, bethanecol, carbachol, oxotremorine, L-689,660 (1-azabicyclo[2.2.2]octane, 3-(6-chloropyrazinyl)maleate), pilocarpine, muscarine, McN-A 343 (4-[[[(3-Chlorophenyl)amino]carbonyl]oxy]-N,N,N-trimethyl-2-butyn-1-aminium chloride), 77-LH-218-1 (1-[3-(4-Butyl-1-piperidinyl)propyl]-3,4-dihydro-2(1H)-quinolinone) and methacholine.

In some embodiments, the muscarinic acetylcholine receptor agonist is carbachol.

In some embodiments the AP-1 stimulant (e.g. the muscarinic acetylcholine receptor agonist) is present in the differentiation medium of the invention at a concentration of 0.001-500 μM, 0.01-500 μM, 0.01-250 μM, 0.01-150 μM, 0.1-500 μM, 0.1-100 μM, 0.5-500 μM, 0.5-100 μM, 0.5-50 μM, 1-500 μM, 1-300 μM, 1-200 μM, 1-20 μM, 1-5 μM, 10-300 μM, 50-300 μM, 50-200 μM or 50-150 μM.

In some embodiments, the differentiation medium of the invention comprises carbachol at a concentration of 0.001-500 μM, 0.001-300 μM, 0.01-500 μM, 0.01-300 μM, 0.1-500 μM, 0.1-300 M, 1-500 μM, 10-500 μM, 100-500 μM, 1-300 μM, 10-300 μM, 50-300 μM, 50-200 μM or 50-150 μM. For example, in some embodiments, the differentiation medium of the invention comprises carbachol at a concentration of 100 μM.

### Muscarinic Acetylcholine Receptor Agonists

As explained above, it was previously found that inclusion of a muscarinic acetylcholine receptor agonist, carbachol, in a differentiation medium promotes differentiation. Accordingly, in some embodiments, the differentiation medium of the invention further comprises a muscarinic acetylcholine receptor agonist.

Any suitable muscarinic acetylcholine receptor agonist may be used. In some embodiments, the muscarinic acetylcholine receptor agonist is an M₃ muscarinic acetylcholine receptor agonist (e.g. acetylcholine, bethanechol, carbachol, oxotremorine or pilocarpine).

In some embodiments, the muscarinic acetylcholine receptor agonist is selected from one or more of the following: acetylcholine, bethanecol, carbachol, oxotremorine, L-689,660 (1-azabicyclo[2.2.2]octane, 3-(6-chloropyrazinyl)maleate), pilocarpine, muscarine, McN-A 343 (4-[[[(3-Chlorophenyl)amino]carbonyl]oxy]-N,N,N-trimethyl-2-butyn-1-aminium chloride), 77-LH-218-1 (1-[3-(4-Butyl-1-piperidinyl)propyl]-3,4-dihydro-2(1H)-quinolinone) and methacholine.

In some embodiments, the muscarinic acetylcholine receptor agonist is carbachol.

In some embodiments the AP-1 stimulant (e.g. the muscarinic acetylcholine receptor agonist) is present in the differentiation medium of the invention at a concentration of 0.001-500 μM, 0.01-500 μM, 0.01-250 μM, 0.01-150 μM, 0.1-500 μM, 0.1-100 μM, 0.5-500 μM, 0.5-100 μM, 0.5-50 μM, 1-500 μM, 1-300 μM, 1-200 μM, 1-20 μM, 1-5 μM, 10-300 μM, 50-300 μM, 50-200 μM or 50-150 μM.

In some embodiments, the differentiation medium of the invention comprises carbachol at a concentration of 0.001-500 μM, 0.001-300 μM, 0.01-500 μM, 0.01-300 μM, 0.1-500 μM, 0.1-300 M, 1-500 μM, 10-500 μM, 100-500 μM, 1-300 μM, 10-300 μM, 50-300 μM, 50-200 μM or 50-150 μM. For example, in some embodiments, the differentiation medium of the invention comprises carbachol at a concentration of 100 μM.

### Carbachol Agonist

As explained above, it was previously found that inclusion of a muscarinic acetylcholine receptor agonist, carbachol, in a differentiation medium promotes differentiation. Accordingly, in some embodiments, the differentiation medium of the invention further comprises carbachol or a carbachol agonist. A carbachol agonist is defined herein as being an agent that shares one or more biological activities with carbachol.

cAMP Pathway Activators

In some embodiments, the differentiation medium of the invention further comprises a cAMP pathway activator. The cAMP pathway activator may be any suitable activator which increases the levels of cAMP in a cell. The cAMP pathway involves activation of many types of hormone and neurotransmitter G-protein coupled receptors. Binding of the hormone or neurotransmitter to its membrane-bound receptor induces a conformational change in the receptor that leads to activation of the α-subunit of the G-protein. The activated G subunit stimulates, while the non-activated G subunit inhibits adenylyl cyclase. Stimulation of adenylyl cyclase catalyzes the conversion of cytoplasmic ATP to cAMP thus increasing the levels of cAMP in the cell. Therefore, the cAMP pathway activator may, for example, be an adenylyl cyclase activator. Examples of suitable adenylyl cyclase activators include forskolin, a forskolin analogue and cholera toxin. In some embodiments, the cAMP pathway activator is forskolin. In some embodiments, the cAMP pathway activator is not cholera toxin. In some embodiments the cAMP pathway activator may be a cAMP analog, for example 8-bromo-cAMP. 8-bromo-cAMP is a cell-permeable cAMP analog having greater resistance to hydrolysis by phosphodiesterases than cAMP. In some embodiments, the cAMP pathway activator is NKH477 (e.g. catalogue no. Tocris 1603).

cAMP pathway activators can be identified using methods known in the art, for example, using a competitive immunoassay which measures cAMP levels. The CatchPoint® Cyclic-AMP Fluorescent Assay Kit (Molecular Devices LLC) is an example of a commercially available kit for carrying out such an immunoassay. The cAMP in the sample or standard competes with horseradish peroxidase (HRP)-labeled cAMP conjugate for binding sites on the anti-cAMP antibodies. In the absence of cAMP, most of the HRP-cAMP conjugate is bound to the antibody. Increasing concentrations of cAMP competitively decrease the amount of bound conjugate, thus decreasing measured HRP activity. A cAMP pathway activator would result in increased levels of cAMP and decreased measured HRP activity, compared to a control.

In some embodiments, the cAMP pathway activator is used at a concentration of between about 10 nM to about 500 μM, about 10 nM to about 100 μM, about 1 μM to about 50 μM, about 1 μM to about 25 μM, about 5 μM to about 1000 μM, about 5 μM to about 500 μM, about 5 μM to about 100 μM, about 5 μM to about 50 μM, about 5 μM to about 25 μM, about 10 μM to about 1000 μM, about 10 μM to about 500 μM, about 10 μM to about 100 μM, about 10 μM to about 50 μM, about 10 μM to about 25 μM, or about 20 μM. In some embodiments the cAMP pathway activator is used at a concentration of at least 10 nM, 20 nM, 50 nM, 100 nM, 200 nM, 500 nM, 1 μM, at least 2 μM, at least 5 μM, at least 10 μM, at least 20 μM, at least 30 μM, at least 50 μM, or at least 100 μM.

The concentration selected may depend upon the cAMP pathway activator used and can be determined by the person skilled in the art depending upon the potency of the cAMP pathway activator. For example, NKH477 is generally more potent than 8-BR-cAMP and forskolin. A more potent cAMP pathway activator can be used at lower concentrations to the same effect.

For example, NKH477 can in some embodiments be used at a concentration of between about 100 nM and about 10 μM, or at a concentration of about 100 nM, about 1 μM or about 10 μM. 8-BR-cAMP or forskolin can in some embodiments be used at a concentration of between about 1 μM and about 100 μM, or at a concentration of about 1 μM, about 10 μM or about 100 μM.

Cholera toxin can in some embodiments be used at a concentration of between about 1 ng/ml and about 500 ng/ml, about 10 ng/ml and about 100 ng/ml, about 50 ng/ml and about 100 ng/ml, or about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 200 ng/ml, about 300 ng/ml, about 400 ng/ml or about 500 ng/ml.

### Supplements

The differentiation medium of the invention is preferably supplemented with one or more (e.g. 1, 2, 3 or all) of the compounds selected from the group consisting of B27, N-acetylcysteine and N2. Thus in some embodiments the differentiation medium further comprises one or more components selected from the group consisting of: B27, N2 and N-Acetylcysteine. For example, in some embodiments, the differentiation medium further comprises B27, N-Acetylcysteine and N2.

B27 (Invitrogen), N-Acetylcysteine (Sigma) and N2 (Invitrogen), and Nicotinamide (Sigma) are believed to control proliferation of the cells and assist with DNA stability.

In some embodiments, N-Acetylcysteine is present in the differentiation medium at a concentration of 0.1-200 mM, 0.1-100 mM, 0.1-50 mM, 0.1-10 mM, 0.1-5 mM, 0.5-200 mM, 0.5-100 mM, 0.5-50 mM, 0.5-10 mM, 0.5-5 mM, 1-100 mM, 1-50 mM, 1-10 mM, 1-5 mM. In some embodiments, N-Acetylcysteine is present in the differentiation medium at a concentration of about 1.25 mM.

In some embodiments, the B27 supplement is ‘B27 Supplement minus Vitamin A’ (also referred to herein as “B27 without Vitamin A” or “B27 wo VitA”; available from Invitrogen, Carlsbad, CA; www.invitrogen.com; currently catalog no. 12587010; and from PAA Laboratories GmbH, Pasching, Austria; www.paa.com; catalog no. F01-002; Brewer et al. (1993) J Neurosci Res. 35(5):567-76). In some embodiments, the B27 supplement can be replaced with a generic formulation that comprises one or more of the components selected from the list: biotin, cholesterol, linoleic acid, linolenic acid, progesterone, putrescine, retinyl acetate, sodium selenite, tri-iodothyronine (T3), DL-alpha tocopherol (vitamin E), albumin, insulin and transferrin.

The B27 Supplement supplied by PAA Laboratories GmbH comes as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists. B27 Supplement may be added to a differentiation medium as a concentrate or diluted before addition to a differentiation medium. It may be used at a 1× final concentration or at other final concentrations (e.g. 0.1× to 4× concentration, 0.1× to 2× concentration, 0.5× to 2× concentration, 1× to 4× concentration, or 1× to 2× concentration). Use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into a differentiation medium of the invention. It is also envisaged that some or all of these components may be added separately to the differentiation medium instead of using the B27 Supplement. Thus, the differentiation medium may comprise some or all of these components.

In some embodiments, retinoic acid is absent from the B27 Supplement used in the differentiation medium, and/or is absent from the differentiation medium.

‘N2 Supplement’ (also referred to herein as “N2”) is available from Invitrogen, Carlsbad, CA; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979. The N2 Supplement supplied by PAA Laboratories GmbH comes as a 100× liquid concentrate, containing 500 g/ml human transferrin, 500 g/ml bovine insulin, 0.63 μg/ml progesterone, 1611 g/ml putrescine, and 0.52 g/ml sodium selenite. N2 Supplement may be added to a differentiation medium as a concentrate or diluted before addition to a differentiation medium. It may be used at a 1× final concentration or at other final concentrations (e.g. 0.1× to 4× concentration, 0.1× to 2× concentration, 0.5× to 2× concentration, 1× to 4× concentration, or 1× to 2× concentration). Use of N2 Supplement is a convenient way to incorporate transferrin, insulin, progesterone, putrescine and sodium selenite into a differentiation medium of the invention. It is of course also envisaged that some or all of these components may be added separately to the differentiation medium instead of using the N2 Supplement. Thus, the differentiation medium may comprise some or all of these components.

In some embodiments in which the medium comprises B27, it does not also comprise N2. The embodiments of the present invention can therefore be adapted to exclude N2 when B27 is present, if desired.

In some embodiments N2 is not present in the differentiation medium.

In some embodiments in which the medium comprises N2, it does not also comprise B27. The embodiments of the present invention can therefore be adapted to exclude B27 when N2 is present, if desired.

In some embodiments B27 is not present in the differentiation medium.

In some embodiments the differentiation medium is supplemented with B27 and/or N2.

In some embodiments, the basal medium is supplemented with 150 ng/ml to 250 ng/ml N-Acetylcysteine; preferably, the basal medium is supplemented with about 200 ng/ml N-Acetylcysteine.

Any suitable pH may be used. For example, the pH of the medium may be in the range from about 7.0 to 7.8, in the range from about 7.2 to 7.6, or about 7.4. The pH may be maintained using a buffer. A suitable buffer can readily be selected by the skilled person. Buffers that may be used include carbonate buffers (e.g. NaHCO₃), and phosphates (e.g. NaH₂PO₄). These buffers are generally used at about 50 to about 500 mg/l. Other buffers such as N-[2-hydroxyethyl]-piperazine-N′-[2-ethanesul-phonic acid](HEPES) and 3-[N-morpholino]-propanesulfonic acid (MOPS) may also be used, normally at around 1000 to around 10,000 mg/l. In some embodiments, the buffer is selected from one or more of the list: phosphate buffer (e.g. KH₂PO₄, K₂HPO₄, Na₂HPO₄, NaCl, NaH₂PO₄) acetate buffer (e.g. HOAc or NaOAc), citrate buffer (e.g. Citric acid or Na-citrate), or a TRIS buffer (e.g. TRIS, TRIS-HCl) or an organic buffer. In some embodiments, the organic buffer is a zwitterionic buffer, such as a Good's buffer, e.g. selected from HEPES, MOPS, MES, ADA, PIPES, ACES, MOPSO, Cholamine Chloride, BES, TES, DIPSO, acetamindoglycine, TAPSO, POPSO, HEPPSO, HEPPS, Tricine, Glycinamide, Bicine, TAPS, AMPSO, CABS, CHES, CAPS and CAPSO. A preferred buffer is HEPES, e.g. at a concentration of 0.1-100 mM, 0.1-50 mM, 0.5-50 mM, 1-50 mM, 1-20 mM. or 5-15 mM. In some embodiments, HEPES is added to the culture medium at about 10 mM. A differentiation medium may also comprise a pH indicator, such as phenol red, to enable the pH status of the medium to be easily monitored (e.g. at about 5 to about 50 mg/litre).

A differentiation medium for use in the invention may comprise one or more amino acids. The skilled person understands the appropriate types and amounts of amino acids for use in differentiation media. Amino acids which may be present include L-alanine, L-arginine, L-asparagine, L-aspartic acid, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine and combinations thereof. Some differentiation media will contain all of these amino acids. Generally, each amino acid when present is present at about 0.001 to about 1 g/L of medium (usually at about 0.01 to about 0.15 g/L), except for L-glutamine which is present at about 0.05 to about 1 g/L (usually about 0.1 to about 0.75 g/L). The amino acids may be of synthetic origin.

A differentiation medium for use in the invention may comprise one or more vitamins. The skilled person understands the appropriate types and amounts of vitamins for use in differentiation media. Vitamins which may be present include thiamine (vitamin B1), riboflavin (vitamin B2), niacin (vitamin B3), D-calcium pantothenate (vitamin B5), pyridoxal/pyridoxamine/pyridoxine (vitamin B6), folic acid (vitamin B9), cyanocobalamin (vitamin B12), ascorbic acid (vitamin C), calciferol (vitamin D2), DL-alpha tocopherol (vitamin E), biotin (vitamin H) and menadione (vitamin K).

A differentiation medium for use in the invention may comprise one or more inorganic salts. The skilled person understands the appropriate types and amounts of inorganic salts for use in differentiation media. Inorganic salts are typically included in differentiation media to aid maintenance of the osmotic balance of the cells and to help regulate membrane potential. Inorganic salts which may be present include salts of calcium, copper, iron, magnesium, potassium, sodium, zinc. The salts are normally used in the form of chlorides, phosphates, sulphates, nitrates and bicarbonates. Specific salts that may be used include CaCl₂), CuSO₄-5H₂O, Fe(NO₃)-9H₂O, FeSO₄-7H₂O, MgCl, MgSO₄, KCl, NaHCO₃, NaCl, Na₂HPO₄, Na₂HPO₄—H₂O and ZnSO₄-7H₂O.

The osmolarity of the medium may be in the range from about 200 to about 400 mOsm/kg, in the range from about 290 to about 350 mOsm/kg, or in the range from about 280 to about 310 mOsm/kg. The osmolarity of the medium may be less than about 300 mOsm/kg (e.g. about 280 mOsm/kg).

A differentiation medium for use in the invention may comprise a carbon energy source, in the form of one or more sugars. The skilled person understands the appropriate types and amounts of sugars to use in differentiation media. Sugars which may be present include glucose, galactose, maltose and fructose. The sugar is preferably glucose, particularly D-glucose (dextrose). A carbon energy source will normally be present at between about 1 and about 10 g/L.

A differentiation medium of the invention may contain serum. Serum obtained from any appropriate source may be used, including fetal bovine serum (FBS), goat serum or human serum. Preferably, human serum is used. Serum may be used at between about 1% and about 30% by volume of the medium, according to conventional techniques.

In other embodiments, a differentiation medium of the invention may contain a serum replacement. Various different serum replacement formulations are commercially available and are known to the skilled person. Where a serum replacement is used, it may be used at between about 1% and about 30% by volume of the medium, according to conventional techniques.

In other embodiments, a differentiation medium of the invention may be serum-free and/or serum replacement-free. A serum-free medium is one that contains no animal serum of any type. Serum-free media may be preferred to avoid possible xeno-contamination of the stem cells. A serum replacement-free medium is one that has not been supplemented with any commercial serum replacement formulation.

In a preferred embodiment, the differentiation medium is supplemented with a purified, natural, semi-synthetic and/or synthetic growth factor and does not comprise an undefined component, such as fetal bovine serum or fetal calf serum. For example, supplements such as B27 (Invitrogen), N-Acetylcysteine (Sigma) and N2 (Invitrogen) stimulate proliferation of some cells. In some embodiments, the differentiation medium is supplemented with one or more of these supplements, for example one, any two or all three of these supplements.

A differentiation medium for use in the invention may comprise one or more trace elements, such as ions of barium, bromium, cobalt, iodine, manganese, chromium, copper, nickel, selenium, vanadium, titanium, germanium, molybdenum, silicon, iron, fluorine, silver, rubidium, tin, zirconium, cadmium, zinc and/or aluminium.
The medium may comprise a reducing agent, such as beta-mercaptoethanol at a concentration of about 0.1 mM.

A differentiation medium of the invention may comprise one or more additional agents, such as nutrients or growth factors previously reported to improve stem cell culture, such as cholesterol/transferrin/albumin/insulin/progesterone, putrescine, selenite/other factors.

### Compositions and Vessels

The invention also provides a composition or cell culture vessel comprising cells and/or organoids according to any one of the aspects of the invention described above, and a differentiation medium according to any one of the aspects of the invention described above. For example, such a composition or cell culture vessel may comprise any number of cells or organoids cultured according to a method of the invention, in a differentiation medium as described above.

According to a still further aspect of the invention, there is provided a hermetically-sealed vessel containing a differentiation medium of the invention. Hermetically-sealed vessels may be preferred for transport or storage of the differentiation media, to prevent contamination. The vessel may be any suitable vessel, such as a flask, a plate, a bottle, a jar, a vial or a bag.

### Exemplary Differentiation Media of the Invention

In some embodiments, the differentiation medium of the invention comprises: a Wnt inhibitor, a Notch inhibitor and an EGFR inhibitor. In some embodiments, the differentiation medium further comprises a basal medium.

Accordingly, in some embodiments the differentiation medium of the invention comprises: a Wnt inhibitor, a Notch inhibitor and an EGFR inhibitor (e.g. Gefitinib). For example, in some embodiments, the differentiation medium of the invention comprises IWP-2 (e.g. at a concentration of about 1.5 μM), DAPT (e.g. at a concentration of about 1 mM) and Gefitinib (e.g. at a concentration of about 5 μM).

For example, in some embodiments the differentiation medium of the invention comprises: a Wnt inhibitor, a Notch inhibitor and an EGFR and ErbB2 inhibitor (e.g. Afatinib). For example, in some embodiments, the differentiation medium of the invention comprises IWP-2 (e.g. at a concentration of about 1.5 μM), DAPT (e.g. at a concentration of about 1 mM) and Afatinib (e.g. at a concentration of about 10 μM).

In some embodiments, the differentiation medium of the invention comprises: a Wnt inhibitor, a Notch inhibitor and a RAS-RAF-MAPK pathway inhibitor (e.g. a MEK inhibitor).

For example, in some embodiments, the differentiation medium of the invention comprises IWP-2 (e.g. at a concentration of about 1.5 μM), DAPT (e.g. at a concentration of about 1 mM) and a MEK inhibitor, such as PD0325901 (e.g. at a concentration of about 1.5 μM).

For example, in some embodiments, the differentiation medium of the invention comprises IWP-2 (e.g. at a concentration of about 1.5 μM), DAPT (e.g. at a concentration of about 1 mM) and an ERK inhibitor, such as SCH772984 (e.g. at a concentration of about 10 μM).

In some embodiments, the differentiation medium of the invention further comprises a BMP pathway activator (e.g. BMP4). As discussed above, the inventors surprisingly found that inclusion of a BMP pathway activator may promote secretin-producing cell differentiation and inhibit enterochromaffin cell differentiation. In other embodiments, the differentiation medium of the invention further comprises a BMP inhibitor (e.g. noggin). As discussed above, the inventors surprisingly found that inclusion of a BMP inhibitor may promote GLP-1-producing cell differentiation and inhibit secretin-producing cell differentiation.

Accordingly, in some embodiments, the differentiation medium of the invention comprises: a Wnt inhibitor (e.g. IWP2), a Notch inhibitor (e.g. DAPT), an EGFR inhibitor (e.g. Gefitinib) and a BMP pathway activator (e.g. BMP4). For example, in some embodiments, the differentiation medium of the invention comprises IWP-2 (e.g. at a concentration of about 1.5 μM), DAPT (e.g. at a concentration of about 1 mM), Gefitinib (e.g. at a concentration of about 5 μM) and BMP4 (e.g. at a concentration of about 10 μg/ml). For example, in some embodiments the differentiation medium of the invention comprises: a Wnt inhibitor, a Notch inhibitor, an EGFR and ErbB2 inhibitor (e.g. Afatinib) and a BMP pathway activator (e.g. BMP4). For example, in some embodiments, the differentiation medium of the invention comprises IWP-2 (e.g. at a concentration of about 1.5 μM), DAPT (e.g. at a concentration of about 1 mM), Afatinib (e.g. at a concentration of about 10 μM) and BMP4 (e.g. at a concentration of about 10 μg/ml).

In some embodiments, the differentiation medium of the invention comprises: a Wnt inhibitor, a Notch inhibitor, a RAS-RAF-MAPK pathway inhibitor (e.g. a MEK inhibitor) and a BMP pathway activator, such as BMP4 (e.g. at a concentration of about 10 μg/ml). For example, in some embodiments, the differentiation medium of the invention comprises IWP-2 (e.g. at a concentration of about 1.5 μM), DAPT (e.g. at a concentration of about 1 mM), a MEK inhibitor, such as PD0325901 (e.g. at a concentration of about 1.5 μM) and a BMP pathway activator, such as BMP4 (e.g. at a concentration of about 10 g/ml). For example, in some embodiments, the differentiation medium of the invention comprises IWP-2 (e.g. at a concentration of about 1.5 μM), DAPT (e.g. at a concentration of about 1 mM), an ERK inhibitor, such as SCH772984 (e.g. at a concentration of about 10 μM) and a BMP pathway activator, such as BMP4 (e.g. at a concentration of about 10 μg/ml).

In some embodiments, the differentiation medium of the invention comprises: a Wnt inhibitor (e.g. IWP2), a Notch inhibitor (e.g. DAPT), an EGFR inhibitor (e.g. Gefitinib) and a BMP inhibitor (e.g. noggin). For example, in some embodiments, the differentiation medium of the invention comprises IWP-2 (e.g. at a concentration of about 1.5 μM), DAPT (e.g. at a concentration of about 1 mM), Gefitinib (e.g. at a concentration of about 5 μM) and noggin (e.g. at a concentration of about 100 ng/ml). For example, in some embodiments the differentiation medium of the invention comprises: a Wnt inhibitor, a Notch inhibitor, an EGFR and ErbB2 inhibitor (e.g. Afatinib) and a BMP inhibitor (e.g. noggin). For example, in some embodiments, the differentiation medium of the invention comprises IWP-2 (e.g. at a concentration of about 1.5 μM), DAPT (e.g. at a concentration of about 1 mM), Afatinib (e.g. at a concentration of about 10 μM) and noggin (e.g. at a concentration of about 100 ng/ml).

In some embodiments, the differentiation medium of the invention comprises: a Wnt inhibitor, a Notch inhibitor, a RAS-RAF-MAPK pathway inhibitor (e.g. a MEK inhibitor) and a BMP inhibitor, such as noggin (e.g. at a concentration of about 100 ng/ml). For example, in some embodiments, the differentiation medium of the invention comprises IWP-2 (e.g. at a concentration of about 1.5 μM), DAPT (e.g. at a concentration of about 1 mM), a MEK inhibitor, such as PD0325901 (e.g. at a concentration of about 1.5 μM) and a BMP inhibitor, such as noggin (e.g. at a concentration of about 100 ng/ml). For example, in some embodiments, the differentiation medium of the invention comprises IWP-2 (e.g. at a concentration of about 1.5 μM), DAPT (e.g. at a concentration of about 1 mM), an ERK inhibitor, such as SCH772984 (e.g. at a concentration of about 10 μM) and a BMP inhibitor, such as noggin (e.g. at a concentration of about 100 ng/ml).

In some embodiments, the differentiation medium of the invention further comprises an mTOR activator, such as MHY1485 (e.g. at a concentration of about 5 μM).

In some embodiments, the differentiation medium of the invention further comprises a Hedgehog inhibitor, such as an SMO inhibitor (e.g. at a concentration of about 5 μM). In some embodiments, the SMO inhibitor is vismogenib.

In preferred embodiments, the differentiation medium further comprises an R-spondin (e.g. at a concentration of about 1 g/ml).

In preferred embodiments, the concentration of EGF in the medium is less than 1 mM.

### Extracellular Matrix

In some embodiments, the methods for differentiating cells comprise culturing cells in contact with an extracellular matrix (ECM). Any suitable ECM may be used. Cells are preferably cultured in a microenvironment that mimics at least in part a cellular niche in which said cells naturally reside. A cellular niche is in part determined by the cells and by an ECM that is secreted by the cells in said niche. A cellular niche may be mimicked by culturing said cells in the presence of biomaterials or synthetic materials that provide interaction with cellular membrane proteins, such as integrins. An ECM as described herein is thus any biomaterial or synthetic material or combination thereof that mimics the in vivo cellular niche, e.g. by interacting with cellular membrane proteins, such as integrins.

In a preferred method of the invention, cells are cultured in contact with an ECM. “In contact” means a physical or mechanical or chemical contact, which means that for separating said resulting organoid or population of epithelial cells from said extracellular matrix a force needs to be used. In some embodiments, the ECM is a three-dimensional matrix. In some embodiment, the cells are embedded in the ECM. In some embodiments, the cells are attached to an ECM. A culture medium of the invention may be diffused into a three-dimensional ECM.

In another embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%. The suspension method may have advantages for upscale methods.

One type of ECM is secreted by epithelial cells, endothelial cells, parietal endoderm-like cells (e.g. Englebreth-Holm-Swarm Parietal Endoderm-Like cells described in Hayashi et al. (2004) Matrix Biology 23:47-62) and connective tissue cells. This ECM comprises of a variety of polysaccharides, water, elastin, and glycoproteins, wherein the glycoproteins comprise collagen, entactin (nidogen), fibronectin, and laminin. Therefore, in some embodiments, the ECM for use in the methods of the invention comprises one or more of the components selected from the list: polysaccharides, elastin, and glycoproteins, e.g. wherein the glycoproteins comprise collagen, entactin (nidogen), fibronectin, and/or laminin. For example, in some embodiments, collagen is used as the ECM. Different types of ECM are known, comprising different compositions including different types of glycoproteins and/or different combination of glycoproteins.

The ECM can be provided by culturing ECM-producing cells, such as for example epithelial cells, endothelial cells, parietal endoderm-like cells or fibroblast cells, in a receptacle, prior to the removal of these cells and the addition of isolated tissue fragments or isolated epithelial cells. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C. These are “naturally-produced ECMs”. Naturally-produced ECMs can be commercially provided. Examples of commercially available extracellular matrices include: extracellular matrix proteins (Invitrogen) and basement membrane preparations from Engelbreth-Holm-Swarm (EHS) mouse sarcoma cells (e.g. Cultrex® Basement Membrane Extract (Trevigen, Inc.) or Matrigel™ (BD Biosciences)).

Therefore, in some embodiments, is a naturally-produced ECM. In some embodiments the ECM is a laminin-containing ECM such as Matrigel™ (BD Biosciences). In some embodiments, the ECM is Matrigel™ (BD Biosciences), which comprises laminin, entactin, and collagen IV. In some embodiments, the ECM comprises laminin, entactin, collagen IV and heparin sulphate proteoglycan (e.g. Cultrex® Basement Membrane Extract Type 2 (Trevigen, Inc.)). In some embodiments, the ECM comprises at least one glycoprotein, such as collagen and/or laminin. A preferred ECM for use in a method of the invention comprises collagen and laminin. A further preferred ECM comprises laminin, entactin, and collagen IV. Mixtures of naturally-produced or synthetic ECM materials may be used, if desired.

In another embodiment, the ECM may be a synthetic ECM. For instance, a synthetic ECM, such as ProNectin (Sigma Z378666) may be used. In a further example, the ECM may be a plastic, e.g. a polyester, or a hydrogel. In some embodiments, a synthetic matrix may be coated with biomaterials, e.g. one or more glycoprotein, such as collagen or laminin.

A three-dimensional ECM supports culturing of three-dimensional epithelial organoids. The extracellular matrix material will normally be a drop on the bottom of the dish in which cells are suspended. Typically, when the matrix solidifies at 37° C., the medium is added and diffuses into the ECM. The cells in the medium stick to the ECM by interaction with its surface structure, for example interaction with integrins.

The culture medium and/or cells may be placed on, embedded in or mixed with the ECM.

For instance, in some embodiments, the single cell, population of cells, or tissue fragment is embedded in Matrigel™, which is optionally growth factor reduced and/or phenol red-free.

In some embodiments, the culture medium is placed on top of the ECM. The culture medium can then be removed and replenished as and when required. In some embodiments, the culture medium is replenished every 1, 2, 3, 4, 5, 6 or 7 days. If components are “added” or “removed” from the media, then this can in some embodiments mean that the media itself is removed from the ECM and then a new media containing the “added” component or with the “removed” component excluded is placed on the ECM.

### Progenitor and Stem Cells for Culture and Obtaining Said Cells

The differentiation methods are useful for progenitor cells. Progenitor cells are defined herein as any cells that have differentiation potential. The term “progenitor cells” therefore encompasses stem cells, including, but not limited to adult stem cells, embryonic stem cells and iPS cells. The progenitor cells may be, for example, primary stem cells or expanded stem cells or part-differentiated stem cells. In some embodiments, the progenitor cells are primary cells, meaning obtained directly from live tissue. In other embodiments, the progenitor cells are secondary cells, i.e. cells that have been cultured and/or passaged. In some embodiments, the progenitor cells are expanded cells. The term “expanded” means that the cells have been cultured in vitro in a culture medium that promotes expansion (e.g. proliferation) of cells in preference to differentiation.

In a preferred embodiment, the progenitor cells are adult progenitor cells, i.e. are derived from adult tissue. In some embodiments, the adult progenitor cells are adult stem cells (e.g. adult epithelial stem cells). In this context “adult” includes newly-born baby or child but excludes embryonic or foetal. In some embodiments the progenitor cells are not derived from embryonic stem cells or embryonic stem cell lines, for example human embryonic stem cells or human embryonic stem cell lines.

In a preferred embodiment, the progenitor cells are epithelial progenitor cells. For example, in a preferred embodiment, the progenitor cells are derived from epithelial tissue, more preferably adult epithelial tissue. Epithelial tissues include the liver, pancreas, intestine, stomach, prostate, lung, breast, ovary, salivary gland, hair follicle, skin, oesophagus, ear, bladder or thyroid. Thus in some embodiments, the progenitor cells are obtained from the liver, pancreas, intestine, stomach, prostate, lung, breast, ovary, salivary gland, hair follicle, skin, oesophagus, ear, bladder or thyroid. In some embodiments, the progenitor cells are obtained from the pancreas, stomach, lung or intestine. In a preferred embodiment, the progenitor cells are obtained from the intestine.

In a preferred embodiment, the progenitor cells are mammalian cells. For example, in a preferred embodiment, the cells are derived from mammalian tissue. For example, in some embodiments the progenitor cells are human cells. In some embodiments the progenitor cells are from a laboratory animal (e.g. mouse, rabbit, rat, guinea pig), a companion animal (e.g. dog, cat, horse) or a farm animal (e.g. cow, pig, sheep, goat).

Primary cells represent the best experimental models for in vivo situations. In a preferred embodiment of the invention, the progenitor cells are (or are derived in cell culture from) primary progenitor cells (e.g. primary epithelial stem cells). Primary cell cultures can be passaged to form secondary cell cultures. With the exception of cancer cells, traditional secondary epithelial cell cultures have a limited lifespan. After a certain number of population doublings (e.g. 50-100 generations) cells undergo the process of senescence and stop dividing. Cells from secondary cultures can become immortalized to become continuous cell lines. Immortalization can occur spontaneously, or may be virally- or chemically-induced. Immortalized cell lines are also known as transformed cells. In a preferred embodiment of the invention, the cells are obtained from expanded epithelial stem cell cultures, preferably expanded organoids, which have been expanded and/or passaged without immortalisation or transformation. In some embodiments, these expanded epithelial cultures or organoids may be genetically heterogeneous (unlike traditional cell lines). Thus in some embodiments, the progenitor cells are not immortalised or transformed cells or are not derived from an immortalised cell line or a transformed cell line.

The progenitor cells may be obtained by any suitable method, for example, as described in WO 2010/090513, WO 2012/014076, WO 2012/168930 or WO 2015/173425. In some embodiments, cells are isolated by collagenase digestion, for example, as described in Dorell et al., 2008 (Hepatology. 2008 October; 48(4):1282-91. Surface markers for the murine oval cell response. Dorrell C, Erker L, Lanxon-Cookson K M, Abraham S L, Victoroff T, Ro S, Canaday P S, Streeter P R, Grompe M). In some embodiments, collagenase digestion is performed on a tissue biopsy. In some embodiments, collagenase and accutase digestion are used to obtain the progenitor cells for use in the invention.

In some embodiments, progenitor cell is an epithelial stem cell that expresses Lgr5. An organoid is preferably obtained using a cell from an adult tissue, preferably an epithelial stem cell from an adult tissue, more preferably an epithelial stem cell that expresses Lgr5.

In a most preferred embodiment, progenitor cells are or comprise adult epithelial stem cells expressing Lgr5.

In some embodiments the progenitor cells are normal cells, meaning that the cells have a normal karyotype, genotype and/or phenotype. In alternative embodiments, the progenitor cells are disease cells, meaning that they have a disease karyotype, genotype and/or phenotype. For example, in some embodiments, the progenitor cells are cancer cells. Thus, it is envisaged, for example, that the epithelial stem cells may be Lgr5 positive cancer stem cells. Accordingly, the cells may be obtained from a tumour, if required. In alternative embodiments, the progenitor cells are diseased progenitor cells, for example progenitor cells infected with intracellular pathogens (e.g. bacteria, viruses or parasites).

### Exemplary Methods

The invention provides a method for differentiating progenitor cells, wherein said method comprises culturing the cells in a differentiation medium described herein. In a preferred embodiment, the cells are cultured in contact with an ECM as described herein.

The invention also provides a method for culturing progenitor cells, wherein said method comprises culturing the cells in an expansion medium and subsequently culturing the cells in a differentiation medium described herein. In some embodiments, the cells are cultured in contact with an ECM as described herein during the expansion and/or differentiation steps. In some embodiments, the expansion medium is removed prior to culturing the cells in the differentiation medium, e.g. by repeated washings or by splitting the cell culture.

The invention provides a method for culturing a single epithelial stem cell, a population of epithelial stem cells, or an isolated tissue fragment, preferably to obtain an organoid, wherein the method comprises:
culturing an epithelial stem cell, a population of epithelial stem cells or an isolated tissue fragment in an expansion medium to provide an expanded cell population;
optionally inducing quiescence of the expanded cell population (e.g. by treatment with one or more EGFR pathway inhibitors); and
culturing the expanded cell population in a differentiation medium.

The invention also provides a method for differentiating a single progenitor cell or a population of progenitor cells, wherein the method comprises:
culturing an progenitor cell or a population of progenitor cells in a differentiation medium.

The differentiation medium may be any differentiation medium described herein. For example, in some embodiments, the differentiation medium comprises: a Wnt inhibitor (e.g. IWP-2), a Notch inhibitor (e.g. DAPT) and a EGFR pathway inhibitor (e.g. one or more of Gefitinib, Afatinib, PD0325901 and SCH772984). This differentiation medium is particularly suitable for use in a method of obtaining a population of cells enriched in EECs. Markers characteristic of EECs include Chga, Chgb, Tac1, Tph1, Gip, Fabp5, Ghr1, Pyy, Nts, Neurod1, Sst, Sct, cholecystokinin, glucagon and/or pro-glucagon. Further EEC cell-types and their characteristics are described in Grun et al. (2015) Nature 525:251-255. See also Table 2 later. Thus in some embodiments, a differentiation method of the invention results in a population of cells expressing one or more marker selected from Chga, Chgb, Tac1, Tph1, Gip, Fabp5, Ghr1, Pyy, Nts, Neurod1, Sst, Sct, cholecystokinin, glucagon and/or pro-glucagon. In some embodiments the EECs obtained by methods of the invention express serotonin.

In some embodiments, the differentiation medium comprises: a Wnt inhibitor (e.g. IWP-2), a Notch inhibitor (e.g. DAPT) and a EGFR pathway inhibitor (e.g. one or more of Gefitinib, Afatinib, PD0325901 and SCH772984) and a BMP inhibitor (e.g. Noggin or LDN193189). This differentiation medium is particularly suitable for use in a method of obtaining a population of cells enriched in GLP1-secreting EECs. In some embodiments this method results in a population of cells positive for one or more marker selected from Tac1, GLP1 and Chg; and negative for one or more marker selected from secretin, pyy and nts. In some embodiments the population of cells expresses serotonin.

In some embodiments, the differentiation medium comprises: a Wnt inhibitor (e.g. IWP-2), a Notch inhibitor (e.g. DAPT) and a EGFR pathway inhibitor (e.g. one or more of Gefitinib, Afatinib, PD0325901 and SCH772984) and a BMP activator (e.g. BMP4). This differentiation medium is particularly suitable for use in a method of obtaining a population of cells enriched in secretin-secreting EECs. In some embodiments this method results in a population of cells negative for one or more marker selected from Tac1, GLP1 and Chg; and positive for one or more marker selected from secretin, pyy and nts. In some embodiments the population of cells expresses serotonin.

The expansion medium may be any suitable expansion medium for epithelial stem or progenitor cells, preferably a suitable expansion medium for epithelial stem cells (e.g. as described in WO 2010/090513, WO 2012/014076, WO 2012/168930 or WO 2015/173425).

In some embodiments, the expansion medium comprises one or more receptor tyrosine kinase ligands (e.g. EGF, HGF and/or FGF10), nicotinamide and one or more Wnt agonists (e.g. Rspondin conditioned medium and/or Wnt conditioned medium).

In some embodiments, the expansion medium comprises one or more receptor tyrosine kinase ligands (e.g. EGF, HGF and/or FGF10), one or more Wnt agonists (e.g. Rspondin conditioned medium and/or Wnt conditioned medium) and one or more TGF-beta inhibitors (e.g. A83-01).

In some embodiments, the expansion medium comprises one or more receptor tyrosine kinase ligands (e.g. EGF, HGF and/or FGF10), nicotinamide, one or more Wnt agonists (e.g. Rspondin conditioned medium and/or Wnt conditioned medium) and one or more TGF-beta inhibitors (e.g. A83-01).

In any of these embodiments, the expansion medium may further comprise a cAMP pathway activator (e.g. forskolin), gastrin and/or a BMP inhibitor (e.g. Noggin).

For example, in some embodiments (e.g. in a preferred embodiment for the intestine), the expansion medium comprises (i) EGF (e.g. at about 10 to 50 ng/ml); (ii) Noggin conditioned medium (e.g. at about 50 to 100 ng/ml or about 5% final volume); (iii) Rspondin conditioned medium (e.g. at about 1 μg/ml or about 5% final volume). In some embodiments, the expansion medium further comprises n-Acetylcysteine (e.g. at about 1 mM) and 1×B27.

In some embodiments, the expansion medium further comprises valproic acid (e.g. at about 1 mM) and a GSK-3 inhibitor (e.g. at about 3 μM, such as CHIR99021 at about 3 μM). Advantageously, the inclusion of valproic acid and a GSK-3 inhibitor was found to result in a cell population that is enriched in stem cells.

A preferred expansion medium for human organoids comprises (i) EGF (e.g. at about 10 to 50 ng/ml); (ii) Noggin conditioned medium (e.g. at about 50 to 100 ng/ml or about 5% final volume); (iii) Rspondin conditioned medium (e.g. at about 1 μg/ml or about 5% final volume); (iv) a p38 inhibitor (e.g. SB-203580 at a concentration of about 30 μM); (v) a TGF-β inhibitor (e.g. A83-01 at a concentration of about 500 nM); and (vi) Nicotinamide (e.g. at a concentration of about 10 mM).

Preferably, the cells are expanded to generate one or more (e.g. at least 2, 3, 4, 5, 6, 10, 15, 20 or more than 20) organoids prior to differentiation.

In some embodiments, cells are initially cultured in an expansion medium described herein and, once successful organoids have been established, the expansion medium is replaced with a differentiation medium described herein. Accordingly, in some embodiments after one or more (e.g. after two, three, four, five, six, seven, eight, nine, ten or more) passages, the expansion medium is replaced with a differentiation medium. In some embodiments, passaging is carried out weekly. In some embodiments, after two, three, four, five, six, seven, eight, nine, ten or more days, the expansion medium is replaced with a differentiation medium. In a preferred embodiment, the expansion medium is replaced with a differentiation medium after five or more days.

In some embodiments, quiescence of the cells is induced prior to differentiation.

In some embodiments, the epithelial stem cell, population of epithelial stem cells or isolated tissue fragment is washed and plated in an extracellular matrix (e.g. Matrigel) prior to differentiation. In some embodiments, washing is performed with a basal medium or PBS. Without wishing to be bound by any theory, the inventors believe that this washing step is advantageous for differentiation because it removes stem cell factors from the epithelial stem cell, population of epithelial stem cells or isolated tissue fragment.

In some embodiments, the epithelial stem cells population of epithelial stem cells or isolated tissue fragment is cultured in a differentiation medium that comprises a BMP inhibitor (e.g. Noggin) and Rspondin, and that does not comprise EGF, Nicotinamide, a TGFβ inhibitor or Wnt conditioned medium, prior to differentiation in a differentiation medium of the invention. In some embodiments, the culturing in the first differentiation medium is for about one day.

As will be apparent to the skilled reader, the preferred culture methods of the invention are advantageous because feeder cells are not required. Feeder cell layers are often used to support the culture of stem cells, and to inhibit their differentiation. A feeder cell layer is generally a monolayer of cells that is co-cultured with, and which provides a surface suitable for growth of, the cells of interest. The feeder cell layer provides an environment in which the cells of interest can grow. Feeder cells are often mitotically inactivated (e.g. by irradiation or treatment with mitomycin C) to prevent their proliferation. The use of feeder cells is undesirable, because it complicates passaging of the cells (the cells must be separated from the feeder cells at each passage, and new feeder cells are required at each passage). The use of feeder cells can also lead to contamination of the desired cells with the feeder cells. This is clearly problematic for any medical applications, and even in a research context, complicates analysis of the results of any experiments performed on the cells. As noted elsewhere herein, the culture media of the invention are particularly advantageous because they can be used to culture cells without feeder cell contact, i.e. the methods of the invention do not require a layer of feeder cells to support the cells whose growth is being sponsored.

Accordingly, the compositions of the invention may be feeder cell-free compositions. A composition is conventionally considered to be feeder cell-free if the cells in the composition have been cultured for at least one passage in the absence of a feeder cell layer. A feeder cell-free composition of the invention will normally contain less than about 5%, less than about 4%, less than about 3%, less than about 2%, less than about 1% feeder cells (expressed as a % of the total number of cells in the composition) or preferably no feeder cells at all.

In a further aspect, there is provided a method for obtaining a population of differentiated cells or an organoid, wherein the method comprises culturing progenitor cells in a differentiation medium of the invention. Preferably, the method comprises culturing the progenitor cells in a differentiation medium of the invention using a differentiation method as described herein.

In some embodiments, the method comprises obtaining the organoid/population of differentiated cells from a single epithelial stem cell. In another embodiment, the method comprises obtaining the organoid/population of differentiated cells from a population of epithelial stem cells, or from an epithelial tissue fragment.

In a particular embodiment, there is provided a method for obtaining a differentiated organoid, wherein the method comprises culturing epithelial progenitor cells (e.g. epithelial stem cells, optionally expressing Lgr5) in a differentiation medium of the invention, preferably wherein the epithelial progenitor cells are in contact with an ECM, preferably a three-dimensional ECM.

In some embodiments, the method comprises culturing the progenitor cells in an expansion medium for a period of time, for example, 3 days to 10 weeks, 1 to 10 weeks, 1 to 4 weeks or 10 days to 3 weeks, and then passaging the cells (e.g. dissociating the cells to a single cell density, seeding one or more cells at a ratio of 1 cell per container (e.g. per well)), continuing to expand the cells using an expansion medium for a period of time, for example, 3 days to 10 weeks, 1 to 10 weeks, 1 to 4 weeks or 10 days to 3 weeks and repeating the passaging and expanding steps at least once, at least twice, at least three times, at least four times, at least five times, at least six times, at least seven times, at least eight times, at least nine times, at least ten times, at least eleven times, at least twelve times, at least thirteen times or at least fourteen times, prior to differentiating the cells.

In some embodiments, the method comprises culturing the progenitor cells in a differentiation medium for at least 1, at least 2, at least 3, at least 4, at least 5, at least 6, at least 7, at least 8, at least 9, at least 10, at least 11, at least 12, at least 13, at least 14 days. In some embodiments, the method comprises culturing the progenitor cells in a differentiation medium for about 1 to about 20 days, for about 1 to about 10 days or for about 1 to about 5 days.

Following differentiation, the method may further comprise obtaining and/or isolating one or more differentiated cells or a differentiated organoid. For example, following culture of the progenitor cells, it may be useful to remove one or more cells and/or one or more organoids cultured in the culture medium from the culture medium for use in subsequent applications. Any one of a number of physical methods of separation known in the art may be used to select the cells of the invention and distinguish these from other cell types. Such physical methods may involve FACS and various immuno-affinity methods based upon makers specifically expressed by the cells of the invention.

In one embodiment, the cells may be isolated by FACS utilizing an antibody, for example, against one of these markers. As will be apparent to one skilled in the art, this may be achieved through a fluorescent labeled antibody, or through a fluorescent labeled secondary antibody with binding specificity for the primary antibody. Examples of suitable fluorescent labels includes, but is not limited to, FITC, Alexa Fluor® 488, GFP, CFSE, CFDA-SE, DyLight 488, PE, PerCP, PE-Alexa Fluor® 700, PE-Cy5 (TRI-COLOR®), PE-Cy5.5, PI, PE-Alexa Fluor® 750, and PE-Cy7. This list is provided by way of example only, and is not intended to be limiting.

Alternatively, cells may be isolated by immuno-affinity purification, which is a separation method well known in the art. This method relies upon the immobilisation of antibodies on a purification column. The cell sample is then loaded onto the column, allowing the appropriate cells to be bound by the antibodies, and therefore bound to the column. Following a washing step, the cells are eluted from the column using a competitor which binds preferentially to the immobilised antibody, and permits the cells to be released from the column.

It will be apparent to a person skilled in the art that immuno-affinity purification using an immobilised antibody will provide a purified cell population. However, in some embodiments, it may be preferable to purify the cell population further by performing a further round of immuno-affinity purification using one or more of the other identifiable markers and use an aliquot of the isolated clones to ascertain the expression of other relevant intracellular markers.

It will be apparent to a person skilled in the art that the sequential purification steps are not necessarily required to involve the same physical method of separation.

### Differentiated Cells and Organoids

The invention further provides a differentiated organoid or a population of one or more differentiated cells.

A differentiated organoid is a three-dimensional structure comprising differentiated epithelial cell types. A differentiated organoid is typically self-organising, meaning that the three-dimensional arrangement of the cells in the organoid occurs spontaneously as the cells differentiate. In some embodiments, a differentiated organoid is derived from epithelial stem cells, optionally expressing Lgr5.

In one embodiment, the invention provides a differentiated organoid or a population of one or more differentiated cells obtainable or obtained by a method of the invention, for example, which comprises culturing progenitor cells in a differentiation medium of the invention, preferably wherein the progenitor cells are in contact with a three-dimensional ECM.

A ‘population’ of cells is any number of cells greater than 1, but is preferably at least 10 cells, at least 50 cells, at least 100 cells, at least 500 cells, at least 1×10³ cells, at least 1×10⁴ cells, at least 1×10⁵ cells, at least 1×10⁶ cells, at least 1×10⁷ cells, at least 1×10⁸ cells, or at least 1×10⁹ cells. A differentiated organoid according to the present invention may comprise a population of cells of at least 10 cells, at least 50 cells, at least 100 cells, at least 500 cells, at least 1×10³ cells, at least 1×10⁴ cells, at least 1×10⁵ cells, at least 1×10⁶ cells, at least 1×10⁷ cells or more. In some embodiments, each organoid comprises between approximately 1×10³ cells and 5×10³ cells; generally, 10-20 organoids may be grown together in one well, for example of a 24 well plate.

It is clear to the skilled person that an organoid of the invention is not a naturally occurring tissue fragment and/or does not comprise a blood vessel.

Organoids of the invention are, for example, distinguished from naturally occurring tissue because they comprise only epithelial cell types (and not mesenchymal cells or other structural cell types). Thus in some embodiments, the differentiated organoid of the invention comprises only epithelial cells. In some embodiments, the differentiated organoids do not comprise non-epithelial cells. For example, in a particular embodiment, the differentiated organoids do not comprise mesenchymal cells.

The differentiation medium described herein preferably induces or promotes a specific differentiation of cells during at least five days of culture. Differentiation may be measured by detecting the presence of a specific marker associated with the particular tissue lineage, e.g. the enteroendocrine lineage, as defined herein. Differentiation may be measured by detecting the presence of a specific marker associated with the tissue lineage, e.g. the enteroendocrine lineage, as defined herein. Depending on the identity of the marker, the expression of said marker may be assessed by RTPCR or immuno-histochemistry after at least 5, 7, 8, 9, 10, 11, 12, 13, 14, 15, 16 or more days of culture in a differentiation medium as defined herein.

The term “expressed” is used to describe the presence of a marker within a cell. In order to be considered as being expressed, a marker must be present at a detectable level. By “detectable level” is meant that the marker can be detected using one of the standard laboratory methodologies such as PCR, blotting or FACS analysis. A gene is considered to be expressed by a cell of the population of the invention if expression can be reasonably detected after 30 PCR cycles, which corresponds to an expression level in the cell of at least about 100 copies per cell. The terms “express” and “expression” have corresponding meanings. At an expression level below this threshold, a marker is considered not to be expressed. The comparison between the expression level of a marker in a cell of the invention, and the expression level of the same marker in another cell, such as for example an embryonic stem cell, may preferably be conducted by comparing the two cell types that have been isolated from the same species. Preferably this species is a mammal, and more preferably this species is human. Such comparison may conveniently be conducted using a reverse transcriptase polymerase chain reaction (RT-PCR) experiment.

The differentiated organoid of the invention or the population of differentiated cells of the invention preferably comprises at least 50% viable cells, more preferred at least 60% viable cells, more preferred at least 70% viable cells, more preferred at least 80% viable cells, more preferred at least 90% viable cells. Viability of cells may be assessed using Hoechst staining or Propidium Iodide staining in FACS. The viable cells preferably possess corresponding in vivo functions or characteristics. For example, viable enteroendocrine cells preferably possess enteroendocrine functions or characteristics of enteroendocrine cells.

In preferred embodiments, the organoid is an intestine organoid. This means that the organoid is derived from intestine cells.

In preferred embodiments, the population of differentiated cells of the invention is derived from intestine cells.

The inventors have shown that intestine organoids obtained by the methods of the invention are improved because a greater proportion of the cells in these organoids are enteroendocrine cells than in the differentiated intestine organoids previously described in Gran et al. (2015) Nature 525(7568):251-5.

Some of the enteroendocrine cell types found in the mammalian gastrointestinal tract are summarised in Table 2 below.

TABLE 2
Enteroendocrine cells of the mammalian gastrointestinal tract

| | | Luminal | | |
|---|---|---|---|---|
| Cell | Products | receptors | Locations | Principal effects |
| A (X-like) cells and | Ghrelin, nesfatin-1 | T1R1-T1R3; | Stomach | Appetite control, |
| subtypes | | T2Rs | | growth hormone |
| | | | | release |
| ECL cell | Histamine | None (ECL | Stomach | Stimulation of gastric |
| | | cells do not | | acid secretion |
| | | contact the | | |
| | | lumen) | | |
| G cells | Gastrin | LPAR5; | Stomach | Stimulation of gastric |
| | | GPRC6A | | acid secretion |
| D cells | Somatostatin | LPAR5; | Stomach, | Inhibition of gastrin |
| | | GPRC6A | small | release (stomach); |
| | | | intestine | modulation of insulin |
| | | | (and | release (pancreas) |
| | | | pancreas) | |
| Enterochromaffin | 5-HT. 5-HT is also | FFARs 2, 3; | Stomach, | Facilitation of |
| cells | contained in | TRPA1; toxin | small and | intestinal motility |
| | subgroups of I, K | receptors; | large | reflexes and |
| | and L cells | TLRs | intestine | secretion; triggering |
| | | | | of emesis and nausea |
| | | | | in response to toxins |
| I cells | CCK (5-HT) | T2Rs; FFA1; | Proximal | Activation of |
| | | GPR120; | small | gallbladder |
| | | LPAR5; CaSR; | intestine | contraction and |
| | | TRPA1; TLRs | | stimulation of |
| | | | | pancreatic enzyme |
| | | | | secretion |
| K cells, and | GIP | GPR119, | Proximal | Stimulation of insulin |
| subtypes | | GPR120; | small | release |
| | | FFAR1 | intestine | |
| L cells, and | GLP-1, GLP-2, PYY, | T2Rs; T1R2- | Distal small | Stimulation of |
| subtypes | oxyntomodulin (5- | T1R3; FFARs | intestine, | carbohydrate uptake, |
| | HT) | 1-3; GPR119, | colon | slowing of intestinal |
| | | LPAR5, | | transit, appetite |
| | | GPR120; CaSR | | regulation, insulin |
| | | | | release |
| M cells | Motilin | Bile receptors | Small | Initiation of migrating |
| | | | intestine | myoelectric complex |
| | | | | in pig, dog and human |
| N cells | Neurotensin | FFARs | Small and | Inhibition of intestinal |
| | | | large | contractions |
| | | | intestine | |
| P cells | Leptin | Nutrient | Stomach | Appetite regulation, |
| | | receptors | | reduction of food |
| | | | | intake; leptin might |
| | | | | also be in chief cells |
| S cells | Secretin | Acid receptor | Proximal | Reduction of acidity in |
| | | | small | upper small intestine |
| | | | intestine | by stimulation of |
| | | | | bicarbonate release |

Markers characteristic of EECs include Chga, Chgb, Tac1, Tph1, Gip, Fabp5, Ghr1, Pyy, Nts, Neurod1, Sst, Sct, cholecystokinin, glucagon and/or pro-glucagon.

Further EEC cell-types and their characteristics are described in Grun et al. (2015) Nature 525:251-255.

In some embodiments, the differentiated organoid of the invention is a differentiated intestine organoid in which at least 10%, at least 20%, at least 30%, at least 40%, at least 50%, at least 60%, at least 70%, at least 80%, at least 90% or at least 99% of the cells express enteroendocrine cell markers (e.g. Chga, Chgb, Tac1, Tph1, Gip, Fabp5, Ghr1, Pyy, Nts, Neurod1, Sst, Sct, cholecystokinin, glucagon and/or pro-glucagon). In some embodiments, mRNA expression is measured by single-cell RNA sequencing analysis. In some embodiments, the differentiated organoid of the invention is a differentiated intestine organoid in which at least 10%, at least 20%, at least 30%, at least 40%, at least 50%, at least 60%, at least 70%, at least 80%, at least 90% or at least 99% of the cells express an enteroendocrine cell marker characteristic of a particular enteroendocrine cell type, particularly a cell type described in Table 2. For example, in some embodiments, the enteroendcorine cell marker characteristic of a particular enteroendocrine cell type is Gcg and/or GLP-1. Gcg is the gene expressing preproglucagon from which GLP-1 is derived. In other embodiments, the enteroendocrine cell marker characteristic of a particular enteroendocrine cell type is Sct.

In some embodiments, the differentiated organoid of the invention is a differentiated intestine organoid in which at least 10%, at least 20%, at least 30%, at least 40%, at least 50%, at least 60%, at least 70%, at least 80%, at least 90% or at least 99% of the cells express one or more (e.g. 2, 3, 4 or more) of the products listed in Table 2.

In some embodiments, the differentiated organoid of the invention is a differentiated intestine organoid in which less than 50%, less than 40%, less than 30%, less than 20%, less than 10% or less than 1% of the cells express Goblet or Paneth cell markers (e.g. Lyz1, Defa6, Agr2, Gob5, Muc2, Ttf3 and/or Defa24). In some embodiments, mRNA expression is measured by single-cell RNA sequencing analysis.

In some embodiments, the differentiated organoid of the invention is a differentiated intestine organoid in which less than 50%, less than 40%, less than 30%, less than 20%, less than 10% or less than 1% of the cells express enterocyte markers (e.g. Aldob, Apoa1 and/or Alpi). In some embodiments, mRNA expression is measured by single-cell RNA sequencing analysis.

In some embodiments, the differentiated organoid of the invention is a differentiated intestine organoid in which less than 50%, less than 40%, less than 30%, less than 20%, less than 10% or less than 1% of the cells express Tuft cell markers (e.g. Dclk1 and/or Trpm5). In some embodiments, mRNA expression is measured by single-cell RNA sequencing analysis.

In some embodiments, the differentiated organoid has a cystic structure with a central lumen. In some embodiments the central lumen is surrounded by an epithelial monolayer.

Also provided is a differentiated organoid or a differentiated population of enteroendocrine cells of the invention in a differentiation medium of the invention.

In one embodiment, there is provided an organoid in a differentiation medium, for example as described herein.

In an embodiment, a differentiated organoid is an organoid which is still being cultured using a method of the invention and is therefore in contact with an extracellular matrix. Preferably, a differentiated organoid is embedded in a non-mesenchymal extracellular matrix.

The organoid or population of progenitor cells may be from any mammalian tissue, but is preferably from a human. In some embodiments, it is from a mouse, rabbit, rat, guinea pig or other non-human mammal.

TABLE 3
differences between differentiated intestine
organoids and primary intestinal tissue

| | Differentiated organoid | Primary tissue |
|---|---|---|
| Cell composition | Approximately 30-80% | Approximately 1% |
| | EEC | EEC. |

### Uses of the Differentiated Organoids

Uses of the organoids described herein and cells derived from the organoids are likewise provided. Where organoids are referred to in this section, these are differentiated organoids in accordance with the invention. It will be understood by the skilled person that may of uses are also applicable to a population of differentiated cells obtained and/or obtainable by the methods of the invention. Such uses of the population of differentiated cells obtained and/or obtainable by the methods of the invention are also provided.

For example, the invention provides the use of a differentiated organoid, or a cell derived from said organoid, in a drug discovery screen; toxicity assay; research of tissue embryology, cell lineages, and differentiation pathways; research to identify the chemical and/or neuronal signals that lead to the release of the respective hormones; gene expression studies including recombinant gene expression; research of mechanisms involved in tissue injury and repair; research of inflammatory and infectious diseases; studies of pathogenetic mechanisms; or studies of mechanisms of cell transformation and aetiology of cancer.

The invention also provides an organoid of the invention, or a cell derived from said organoid, for use in medicine.

The invention also provides an organoid of the invention, or a cell derived from said organoid, for use in treating a disorder, condition or disease.

The invention also provides an organoid of the invention, or a cell derived from said organoid for use in regenerative medicine, for example, wherein the use involves transplantation of the organoid or cell into a patient.

The invention provides the use of an organoid of the invention or cells derived from said organoid in drug screening, (drug) target validation, (drug) target discovery, toxicology and toxicology screens, personalized medicine, regenerative medicine and/or as ex vivo cell/organ models, such as disease models.

Cells and organoids cultured according to the media and methods of the invention are thought to faithfully represent the in vivo situation. This is true both for differentiated populations of cells and organoids grown from normal tissue and for differentiated populations of cells and organoids grown from diseased tissue. Therefore, as well as providing normal ex vivo cell/organ models, the organoids of the invention can be used as ex vivo disease models.

Organoids of the invention can also be used for culturing of a pathogen and thus can be used as ex vivo infection models. Examples of pathogens that may be cultured using an organoid of the invention include viruses, bacteria, prions or fungi that cause disease in its animal host. Thus an organoid of the invention can be used as a disease model that represents an infected state. In some embodiments of the invention, the organoids can be used in vaccine development and/or production.

Diseases that can be studied by the organoids of the invention thus include genetic diseases, metabolic diseases, pathogenic diseases, inflammatory diseases etc., for example including, but not limited to: diabetes (such as type I or type II), cystic fibrosis, carcinomas, adenocarcinomas, adenomas, gastroenteropancreatic neuroendocrine tumours, inflammatory bowel disease (such as Crohn's disease).

Traditionally, cell lines and more recently iPS cells have been used as ex vivo cell/organ and/or disease models (for example, see Robinton et al. Nature 481, 295, 2012). However, these methods suffer a number of challenges and disadvantages. For example, cell lines cannot be obtained from all patients (only certain biopsies result in successful cell lines) and therefore, cell lines cannot be used in personalised diagnostics and medicine. iPS cells usually require some level of genetic manipulation to reprogramme the cells into specific cell fates. Alternatively, they are subject to culture conditions that affect karotypic integrity and so the time in culture must be kept to a minimum (this is also the case for human embryonic stem cells). This means that iPS cells cannot accurately represent the in vivo situation but instead are an attempt to mimic the behaviour of in vivo cells. Cell lines and iPS cells also suffer from genetic instability.

By contrast, the organoids of the invention provide a genetically stable platform which faithfully represents the in vivo situation. In some embodiments, the organoids of the invention comprise all differentiated cell types that are present in the corresponding in vivo situation. In other embodiments, the organoids of the invention may be further differentiated to provide all differentiated cell types that are present in vivo. Thus the organoids of the invention can be used to gain mechanistic insight into a variety of diseases and therapeutics, to carry out in vitro drug screening, to evaluate potential therapeutics, to identify possible targets (e.g. proteins) for future novel (drug) therapy development and/or to explore gene repair coupled with cell-replacement therapy.

The organoids of the invention can be frozen and thawed and put into culture without losing their genetic integrity or phenotypic characteristics and without loss of proliferative capacity. Thus the organoids can be easily stored and transported. Thus in some embodiments, the invention provides a frozen organoid.

For these reason the organoids or differentiated populations of cells of the invention can be a tool for drug screening, target validation, target discovery, toxicology and toxicology screens and personalized medicine.

Accordingly, in a further aspect, the invention provides the use of an organoid or cell derived from said organoid according to the invention in a drug discovery screen, toxicity assay or in medicine, such as regenerative medicine. For example, any one of the intestinal organoids may be used in a drug discovery screen, toxicity assay or in medicine, such as regenerative medicine.

### Mucosal Vaccines

An additional important use of the organoids is in the development of mucosal vaccinations. Mucosal vaccines are vaccines that are administered via the mucosa. This can be any mucosal surface such as via the nose, mouth, or rectum. They can be administered via an inhaler, a spray or other external aids. This has several clear benefits over injections such as that no medical staff are needed for administering the vaccine, which may be important, for example in developing countries.

In the intestine, M cells (or “microfold cells”) are cells found in the follicle-associated epithelium of the aggregated lymphoid nodules of the ileum. They transport organisms and particles from the gut lumen to immune cells across the epithelial barrier, and thus are important in stimulating mucosal immunity. They have the unique ability to take up antigen from the lumen of the small intestine via endocytosis or phagocytosis, and then deliver it via transcytosis to dendritic cells (an antigen presenting cell) and lymphocytes (namely T cells) located in a unique pocket-like structure on their basolateral side. M cells are a type of enteroendocrine cells. As explained above, the inventors have found an improved method for differentiating progenitor cells to an enteroendocrine cell fate. Therefore, the differentiated organoids of the invention are enriched in enteroendocrine cells, such as M cells.

Organoids can in some cases develop into M cells when stimulated with RANK ligand (e.g. see FIG. 49 of WO2012/169830). Therefore, in some embodiments, the differentiation medium further comprises RANK ligand.

The efficiency of mucosal vaccines can be substantially increased when they are targeted to M cells. Therefore, the differentiated cell population or organoid of the invention can be used for testing the ability of M cells to take up pathogens or antigens and to present them to the immune system. Therefore, in some embodiments the invention provides the use of an organoid of the invention in drug screening, for example in vaccine development and/or vaccine production. For example, in some embodiments the organoid may be used for the development or production of vaccines against viral, bacterial, fungal or other parasitic infections, for example (but not limited to) cholera, Respiratory syncytial virus (RSV), Rotavirus and HIV. In a particular embodiment, the invention provides organoids that have been differentiated in a culture medium of the invention, for use in mucosal vaccine development.

### Drug Screening

For preferably high-throughput purposes, said organoid of the invention is cultured in multiwell plates such as, for example, 96 well plates or 384 well plates. Libraries of molecules are used to identify a molecule that affects said organoids. Preferred libraries comprise antibody fragment libraries, peptide phage display libraries, peptide libraries (e.g. LOPAP™, Sigma Aldrich), lipid libraries (BioMol), synthetic compound libraries (e.g. LOP AC™, Sigma Aldrich) or natural compound libraries (Specs, TimTec). Furthermore, genetic libraries can be used that induce or repress the expression of one of more genes in the progeny of the stem cells. These genetic libraries comprise cDNA libraries, antisense libraries, and siRNA or other non-coding RNA libraries. The cells are preferably exposed to multiple concentrations of a test agent for a certain period of time. At the end of the exposure period, the cultures are evaluated. The term “affecting” is used to cover any change in a cell, including, but not limited to, a reduction in, or loss of, proliferation, a morphological change, and cell death. Said organoid of the invention can also be used to identify drugs that specifically target epithelial carcinoma cells, but not said organoid of the invention.

The ability to obtain a useful organoid of the invention in short time periods (days) shows that the organoids would be highly useful for testing individual patient responses to specific drugs and tailoring treatment according to the responsiveness. In some embodiments, wherein the organoid is obtained from a biopsy from a patient, the organoid is cultured for less than 21 days, for example less than 14 days, less than 13 days, less than 12 days, less than 11 days, less than 10 days, less than 9 days, less than 8 days, less than 7 days (etc.).

The organoids are also useful for wider drug discovery purposes (e.g. see WO2013/093812 which describes screening for drugs for cystic fibrosis or cholera). Therefore, in some embodiments, the organoids of the invention could be used for screening for cystic fibrosis drugs. However, it will be understood by the skilled person that the organoids of the invention would be widely applicable as drug screening tools for infectious, inflammatory and neoplastic pathologies of the human gastrointestinal tract and other diseases of the gastrointestinal tract and infectious, inflammatory and neoplastic pathologies and other diseases of other tissues described herein such as pancreas, stomach or lung. In some embodiments the organoids of the invention could be used for screening for cancer drugs.

In some embodiments, the organoids of the invention can be used to test libraries of chemicals, antibodies, natural product (plant extracts), etc. for suitability for use as drugs, cosmetics and/or preventative medicines. For instance, in some embodiments, a cell biopsy from a patient of interest, such as tumour cells from a cancer patient, can be cultured using culture media and methods of the invention and then treated with a a chemical compound or a chemical library. It is then possible to determine which compounds effectively modify, kill and/or treat the patient's cells. This allows specific patient responsiveness to a particular drug to be tested thus allowing treatment to be tailored to a specific patient. Thus, this allows a personalized medicine approach.

The added advantage of using the organoids for identifying drugs in this way is that it is also possible to screen normal organoids (organoids derived from healthy tissue) to check which drugs and compounds have minimal effect on healthy tissue. This allows screening for drugs with minimal off-target activity or unwanted side-effects.

Drugs for any number of diseases can be screened in this way. For example the organoids of the invention can be used for screening for drugs for diabetes, cystic fibrosis, carcinomas, adenocarcinomas, adenomas, gastroenteropancreatic neuroendocrine tumours, inflammatory bowel disease (such as Crohn's disease)etc. The testing parameters depend on the disease of interest. For example, when screening for cancer drugs, cancer cell death is usually the ultimate aim. For cystic fibrosis, measuring the expansion of the organoids in response to the drugs and stimuli of CFTR is of interest. In other embodiments, metabolics or gene expression may be evaluated to study the effects of compounds and drugs of the screen on the cells or organoids of interest.

Therefore, the invention provides a method for screening for a therapeutic or prophylactic pharmaceutical drug or cosmetic, wherein the method comprises:
contacting a differentiated cell population or organoid with a candidate molecule (or a library of candidate molecules,
evaluating said differentiated cell populations or organoid for any effects (e.g. any change in the cell, such as a reduction in or loss of proliferation, a morphological change and/or cell death) or a change in organoid (e.g. the organoid size or motility);
identifying the candidate molecule that causes said effects as a potential drug or cosmetic; and optionally
preparing said candidate molecule as pharmaceutical or cosmetic.

In some embodiments, the invention provides a method for preparing a pharmaceutical or cosmetic, wherein the method comprises:
contacting a differentiated cell population or organoid with a candidate molecule (or a library of candidate molecules,
   evaluating said differentiated cell populations or organoid for any effects (e.g. any change in the cell, such as a reduction in or loss of proliferation, a morphological change and/or cell death) or a change in organoid (e.g. the organoid size or motility);
identifying the candidate molecule that causes said effects as a potential drug or cosmetic; and optionally
preparing said candidate molecule as pharmaceutical or cosmetic.

In some embodiments, computer- or robot-assisted culturing and data collection methods are employed to increase the throughput of the screen. In some embodiments, the organoid is derived from a patient biopsy. In some embodiments, the candidate molecule that causes a desired effect on the cultured differentiated cell population (for example, an organoid) is administered to said patient.

Accordingly, in one aspect, there is provided a method of treating a patient comprising:
(a) obtaining a biopsy from the diseased tissue of interest in the patient;
(b) culturing the biopsy to obtain an organoid;
(c) screening for a suitable drug using a screening method of the invention; and
(d) treating said patient with the drug obtained in step (c).

In some embodiments, the drug or cosmetic is used for treating, preventing or ameliorating symptoms of genetic diseases, metabolic diseases, pathogenic diseases, inflammatory diseases etc, for example including, but not limited to: cystic fibrosis, inflammatory bowel disease (such as Crohn's disease), carcinoma, adenoma, adenocarcinoma, colon cancer, diabetes (such as type I or type II), gastroenteropancreatic neuroendocrine tumours, etc.

In some embodiments, the invention provides methods for screening for drugs for regenerative medicine.

### Target Discovery

In some embodiments, the organoids of the invention can be used for target discovery. Cells of the organoids originating from healthy or diseased tissue may be used for target identification. The organoids of the invention may be used for discovery of drug targets for cystic fibrosis, inflammatory bowel disease (such as Crohn's disease), carcinoma, adenoma, adenocarcinoma, colon cancer, diabetes (such as type I or type II), gastroenteropancreatic neuroendocrine tumours etc. Organoids cultured according to the media and methods of the invention are thought to faithfully represent the in vivo situation. For this reason they can be a tool to find novel (molecular) targets in specific diseases.

To search for a new drug target, a library of compounds (such as siRNA) may be used to transduce the cells and inactivate specific genes. In some embodiments, cells are transduced with siRNA to inhibit the function of a (large) group of genes. Any functional read out of the group of genes or specific cellular function can be used to determine if a target is relevant for the study. A disease-specific read out can be determined using assays well known in the art. For example, cellular proliferation is assayed to test for genes involved in cancer. For example, a Topflash assay as described herein, may be used to detect changes in Wnt activity caused by siRNA inhibition. Where growth reduction or cell death occurs, the corresponding siRNA related genes can be identified by methods known in the art. These genes are possible targets for inhibiting growth of these cells. Upon identification, the specificity of the identified target for the cellular process that was studied will need to be determined by methods well known in the art. Using these methods, new molecules can be identified as possible drug targets for therapy.

### Target and Drug Validation Screens

Patient-specific organoids obtained from diseased and/or normal tissue can be used for target validation of molecules identified in high throughput screens. The same goes for the validation of compounds that were identified as possible therapeutic drugs in high throughput screens. The use of primary patient material differentiated in the organoid culture system can be useful to test for false positives, etc. from high throughput drug discovery cell line studies.

In some embodiments, the organoid of the invention can be used for validation of compounds that have been identified as possible drugs or cosmetics in a high-throughput screen.

### Culturing Pathogens

Furthermore, an organoid of the invention can be used for culturing of a pathogen, such as a norovirus which presently lacks a suitable tissue culture or animal model.

### Regenerative Medicine and Transplantation

The invention provides the use of organoids in regenerative medicine and/or transplantation. The invention also provides methods of treatment wherein the method comprises transplanting an organoid into an animal or human.

Organoids of the invention, such as gastric organoids, intestinal organoids or pancreatic organoids are useful in regenerative medicine, for example in post-radiation and/or post-surgery repair of the intestinal epithelium, in the repair of the intestinal epithelium in patients suffering from inflammatory bowel disease such as Crohn's disease and ulcerative colitis, and in the repair of the intestinal epithelium in patients suffering from short bowel syndrome. Further use is present in the repair of the intestinal epithelium in patients with hereditary diseases of the small intestine/colon. Cultures comprising pancreatic organoids are also useful in regenerative medicine, for example as implants after resection of the pancreas or part thereof and for treatment of diabetes such as diabetes I and diabetes II.

In an alternative embodiment, the organoids or cells isolated from the organoids are reprogrammed into related tissue fates such as, for example, pancreatic cells including pancreatic beta-cells. The culturing methods of the present invention will enable to analyse for factors that trans-differentiate the closely related progenitor cell to a pancreatic cell, including a pancreatic beta-cell or a hepatocyte.

It will be clear to a skilled person that gene therapy can additionally be used in a method directed at repairing damaged or diseased tissue. Use can, for example, be made of an adenoviral or retroviral gene delivery vehicle to deliver genetic information, like DNA and/or RNA to stem cells. A skilled person can replace or repair particular genes targeted in gene therapy. For example, a normal gene may be inserted into a nonspecific location within the genome to replace a nonfunctional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the cells of an organoid or derived from an organoid are ex vivo treated by a gene therapy approach and are subsequently transferred to the mammal, preferably a human being in need of treatment.

Since small biopsies taken from adult donors can be expanded without any apparent limit or genetic harm, the technology may serve to generate transplantable epithelium for regenerative purposes. The fact that organoids can be frozen and thawed and put into culture without losing their 3D structure and integrity and without significant cell death further adds to the applicability of organoids for transplantation purposes. Furthermore, in some embodiments, organoids embedded in, or in contact with, an ECM can be transplanted into a mammal, preferably into a human. In another embodiment, organoids and ECM can be transplanted simultaneously into a mammal, preferably into a human.

The skilled person will understand that an ECM can be used as a 3D scaffold for obtaining tissue-like structures comprising expanded populations of cells or organoids according to the invention. Such structures can then be transplanted into a patient by methods well known in the art. An ECM scaffold can be made synthetically using ECM proteins, such as collagen and/or laminin, or alternatively an ECM scaffold can be obtained by “decellularising” an isolated organ or tissue fragment to leave behind a scaffold consisting of the ECM (for example see Macchiarini et al. The Lancet, Volume 372, Issue 9655, Pages 2023-2030, 2008). In some embodiments, an ECM scaffold can be obtained by decellularising an organ or tissue fragment, wherein optionally said organ or tissue fragment is from the intestine, pancreas, lung or stomach.

The invention provides an organoid of the invention or cells derived from said organoid for use in transplantation into a mammal, preferably into a human. Also provided is a method of treating a patient in need of a transplant comprising transplanting an organoid of the invention or cells derived from said organoid into said patient, wherein said patient is a mammal, preferably a human. In some embodiments, the organoid is further differentiated before transplantation into said patient.

For example, a small biopsy to be taken from an adult donor and expanded by an expansion method and subsequently differentiated according to the invention. Thus the technology provided herein may serve to generate transplantable epithelium for regenerative purposes.

The invention provides a method of treating an insulin-deficiency disorder such as diabetes in a patient, or a patient having a dysfunctional pancreas, comprising transplanting a pancreatic organoid of the invention or cells from a pancreatic organoid of the invention into the patient.

In some embodiments, the cells or organoid do not express or secrete insulin upon transplantation into the patient but differentiate within the patient such that they secrete insulin. For example, the ability to secrete insulin may not be detectable immediately upon transplantation, but may be present by about one month after transplantation, for example, by 6 weeks, 2 months or 3 months after transplantation.

The patient is preferably a human, but may alternatively be a non-human mammal, such as a cat, dog, horse, cow, pig, sheep, rabbit or mouse.

Thus, included within the scope of the invention are methods of treatment of a human or non-human animal patient through cellular therapy. Such cellular therapy encompasses the application of the stem cells or organoids of the invention to the patient through any appropriate means. Specifically, such methods of treatment involve the regeneration of damaged tissue. In accordance with the invention, a patient can be treated with allogeneic or autologous stem cells or organoids. “Autologous” cells are cells which originated from the same organism into which they are being re-introduced for cellular therapy, for example in order to permit tissue regeneration. However, the cells have not necessarily been isolated from the same tissue as the tissue they are being introduced into. An autologous cell does not require matching to the patient in order to overcome the problems of rejection. “Allogeneic” cells are cells which originated from an individual which is different from the individual into which the cells are being introduced for cellular therapy, for example in order to permit tissue regeneration, although of the same species. Some degree of patient matching may still be required to prevent the problems of rejection. Thus in some embodiments the transplantation involves autologous cells. In some embodiments, the transplantation involves allogeneic cells.

Generally the cells or organoids of the invention are introduced into the body of the patient by injection or implantation. Generally the cells will be directly injected into the tissue in which they are intended to act. Alternatively, the cells will be injected through the portal vein. A syringe containing cells of the invention and a pharmaceutically acceptable carrier is included within the scope of the invention. A catheter attached to a syringe containing cells of the invention and a pharmaceutically acceptable carrier is included within the scope of the invention.

The skilled person will be able to select an appropriate method and route of administration depending on the material that is being transplanted (i.e. population of cells, single cells in cell suspension, organoids or fragments of organoids) as well as the organ that is being treated.

As discussed above, organoids or cells of the invention can be used in the regeneration of tissue. In order to achieve this function, cells may be injected or implanted directly into the damaged tissue, where they may multiply and eventually differentiate into the required cell type, in accordance with their location in the body. Alternatively, the organoid can be injected or implanted directly into the damaged tissue. Tissues that are susceptible to treatment include all damaged tissues, particularly including those which may have been damaged by disease, injury, trauma, an autoimmune reaction, or by a viral or bacterial infection. In some embodiments of the invention, the cells or organoids of the invention are used to regenerate the colon, small intestine, lung, pancreas or gastric system.

For example, in one embodiment, the cells or organoids of the invention are injected into a patient using a Hamilton syringe.

The skilled person will be aware what the appropriate dosage of cells or organoids of the invention will be for a particular condition to be treated.

In one embodiment the organoids or cells of the invention, either in solution, in microspheres or in microparticles of a variety of compositions, will be administered into the artery irrigating the tissue or the part of the damaged organ in need of regeneration. Generally such administration will be performed using a catheter. The catheter may be one of the large variety of balloon catheters used for angioplasty and/or cell delivery or a catheter designed for the specific purpose of delivering the cells to a particular local of the body. For certain uses, the cells or organoids may be encapsulated into microspheres made of a number of different biodegradable compounds, and with a diameter of about 15 μm. This method may allow intravascularly administered cells or organoids to remain at the site of damage, and not to go through the capillary network and into the systemic circulation in the first passage. The retention at the arterial side of the capillary network may also facilitate their translocation into the extravascular space.

In another embodiment, the organoids or cells may be retrograde injected into the vascular tree, either through a vein to deliver them to the whole body or locally into the particular vein that drains into the tissue or body part to which the cells or organoids are directed. For this embodiment many of the preparations described above may be used.

In another embodiment, the cells or organoids of the invention may be implanted into the damaged tissue adhered to a biocompatible implant. Within this embodiment, the cells may be adhered to the biocompatible implant in vitro, prior to implantation into the patient. As will be clear to a person skilled in the art, any one of a number of adherents may be used to adhere the cells to the implant, prior to implantation. By way of example only, such adherents may include fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

In another embodiment, the organoids or cells of the invention may be embedded in a matrix, prior to implantation of the matrix into the patient. Generally, the matrix will be implanted into the damaged tissue of the patient. Examples of matrices include collagen based matrices, fibrin based matrices, laminin based matrices, fibronectin based matrices and artificial matrices. This list is provided by way of illustration only, and is not intended to be limiting.

In a further embodiment, the organoids or cells of the invention may be implanted or injected into the patient together with a matrix forming component. This may allow the cells to form a matrix following injection or implantation, ensuring that the cells or organoids remain at the appropriate location within the patient. Examples of matrix forming components include fibrin glue liquid alkyl, cyanoacrylate monomers, plasticizers, polysaccharides such as dextran, ethylene oxide-containing oligomers, block co-polymers such as poloxamer and Pluronics, non-ionic surfactants such as Tween and Triton‘8’, and artificial matrix forming components. This list is provided by way of illustration only, and is not intended to be limiting. It will be clear to a person skilled in the art, that any combination of one or more matrix forming components may be used.

In a further embodiment, the organoids or cells of the invention may be contained within a microsphere. Within this embodiment, the cells may be encapsulated within the centre of the microsphere. Also within this embodiment, the cells may be embedded into the matrix material of the microsphere. The matrix material may include any suitable biodegradable polymer, including but not limited to alginates, Poly ethylene glycol (PLGA), and polyurethanes. This list is provided by way of example only, and is not intended to be limiting.

In a further embodiment, the cells or organoids of the invention may be adhered to a medical device intended for implantation. Examples of such medical devices include stents, pins, stitches, splits, prosthetic joints, artificial skin, and rods. This list is provided by way of illustration only, and is not intended to be limiting. It will be clear to a person skilled in the art, that the cells may be adhered to the medical device by a variety of methods. For example, the cells or organoids may be adhered to the medical device using fibrin, one or more members of the integrin family, one or more members of the cadherin family, one or more members of the selectin family, one or more cell adhesion molecules (CAMs), one or more of the immunoglobulin family and one or more artificial adherents.

This list is provided by way of illustration only, and is not intended to be limiting. It will be clear to a person skilled in the art, that any combination of one or more adherents may be used.

The organoid or population of differentiated cells obtained using a method of the invention have a variety of uses. For example, the invention provides the use of the organoid or population of differentiated cells as described herein in a drug discovery screen; toxicity assay; research of embryology, cell lineages, and differentiation pathways; research to identify the chemical and/or neuronal signals that lead to the release of the respective hormones; gene expression studies including recombinant gene expression; research of mechanisms involved in injury and repair; research of inflammatory and infectious diseases; studies of pathogenetic mechanisms; or studies of mechanisms of cell transformation and aetiology of cancer.

In one aspect, the invention provides the use of an organoid or population of differentiated cells as described herein in a drug discovery screen, toxicity assay or in regenerative medicine. Similarly, the invention provides the use of the progeny of organoids of the invention for these uses.

Toxicity assays may be in vitro assays using an organoid or part thereof or a cell derived from an organoid. Such progeny and organoids are easy to culture and more closely resemble primary epithelial cells than, for example, epithelial cell lines such as Caco-2 (ATCC HTB-37), I-407 (ATCC CCL6), and XBF (ATCC CRL 8808) which are currently used in toxicity assays. It is anticipated that toxicity results obtained with organoids more closely resemble results obtained in patients. A cell-based toxicity test is used for determining organ specific cytotoxicity. Compounds that are tested in said test comprise cancer chemopreventive agents, environmental chemicals, food supplements, and potential toxicants. The cells are exposed to multiple concentrations of a test agent for certain period of time. The concentration ranges for test agents in the assay are determined in a preliminary assay using an exposure of five days and log dilutions from the highest soluble concentration. At the end of the exposure period, the cultures are evaluated for inhibition of growth. Data are analysed to determine the concentration that inhibited end point by 50 percent (TC50).

For example, according to this aspect of the invention, a candidate compound may be contacted with cell or organoid as described herein, and any change to the cells or in activity of the cells may be monitored.

For high-throughput purposes, said organoids are cultured in multiwell plates such as, for example, 96 well plates or 384 well plates. Libraries of molecules are used to identify a molecule that affects said organoids. Preferred libraries comprise antibody fragment libraries, peptide phage display libraries, peptide libraries (e.g. LOPAP™, Sigma Aldrich), lipid libraries (BioMol), synthetic compound libraries (e.g. LOP AC™, Sigma Aldrich) or natural compound libraries (Specs, TimTec). Furthermore, genetic libraries can be used that induce or repress the expression of one of more genes in the progeny of the adenoma cells. These genetic libraries comprise cDNA libraries, antisense libraries, and siRNA or other non-coding RNA libraries. The cells are preferably exposed to multiple concentrations of a test agent for certain period of time. At the end of the exposure period, the cultures are evaluated. The term “affecting” is used to cover any change in a cell, including, but not limited to, a reduction in, or loss of, proliferation, a morphological change, and cell death. Said organoids can also be used to identify drugs that specifically target epithelial carcinoma cells, but not said organoids.

Organoids according to the invention can further replace the use of cell lines such as Caco-2 cells in drug discovery screens and in toxicity assays of potential novel drugs or known drugs or known or novel food supplements.

Furthermore, such organoids can be used for culturing of a pathogen.

The invention further provides a differentiated organoid of the invention or a population of differentiated cells of the invention for use in therapy. Also provided is a differentiation organoid of the invention or a cell derived from said organoid for use in treating a disease or condition as described herein.

Similarly, there is provided a method of treating a disease or condition as described herein comprising administering one or more organoids of the invention, or cell derived from said organoid.

The inventors have also demonstrated successful transplantation of organoids into immunodeficient mice (see example 7 of WO 2012/014076), with transplanted liver organoid-derived cells generating both cholangyocytes and hepatocytes in vivo. Therefore, in one embodiment the invention provides organoids or organoid-derived cells of the invention for transplanting into human or animals.

An advantage of the organoids of the invention is that they can be frozen and later be defrosted without loss of function. This enables cell banking, easy storage and rapid availability for acute use. This could be useful for example, in the preparation of an “off-the-shelf” product, for example, in the case of liver, that might be used for the treatment of acute liver toxicity. Organoids can also be grown from cells or tissue fragments taken as small biopsies from live donors minimising any ethical objections to the treatment. The donor may even be from the patient that is to be treated, which could reduce any negative side-effects associated with transplantation of foreign cells and organs and reduce the need for immunosuppressive drugs.

### Pharmaceutical Formulations

In some embodiments, the invention also provides a pharmaceutical formulation comprising the components of the differentiation medium described herein and a pharmaceutically acceptable diluent and/or excipient. For example, there is provided a pharmaceutical formulation comprising one or more Wnt inhibitors (e.g. IWP-2), one or more EGFR pathway inhibitors (e.g. one or more of Gefitinib, Afatinib, PD0325901 and SCH772984), one or more Notch inhibitors (e.g. DAPT), and a pharmaceutically acceptable diluent and/or excipient. In a preferred embodiment, the pharmaceutical formulation does not comprise a basal medium. In some embodiments, the pharmaceutical formulation does not comprise an extracellular matrix. It is envisaged that such formulations may be suitable for promoting differentiation of stem cells in vivo, e.g. for regenerative therapy. Such formulations may be administered in situ (e.g. at the site of tissue damage) or systemically. Alternatively, the formulations may be formulated so that it is suitable for administration by any administration routes known in the art, for example intravenous, subcutaneous, intramuscular administration, mucosal, intradermal, intracutaneous, oral, and ocular. A pharmaceutical formulation may be thus be in any form suitable for such administration, e.g. a tablet, infusion fluid, capsule, syrup, etc.

In some embodiments there is provided a pharmaceutical formulation comprising one or more Wnt inhibitors (e.g. IWP-2), one or more EGFR pathway inhibitors (e.g. one or more of Gefitinib, Afatinib, PD0325901 and SCH772984), one or more Notch inhibitors (e.g. DAPT), one or more BMP inhibitors (e.g. dorsomorphin or LDN193189) and a pharmaceutically acceptable diluent and/or excipient.

In some embodiments there is provided a pharmaceutical formulation comprising one or more Wnt inhibitors (e.g. IWP-2), one or more EGFR pathway inhibitors (e.g. one or more of Gefitinib, Afatinib, PD0325901 and SCH772984), one or more Notch inhibitors (e.g. DAPT), one or more BMP activators (e.g. BMP4, BMP7 or BMP2) and a pharmaceutically acceptable diluent and/or excipient.

### Methods of Treatment

### Modulating Hormone Levels In Vivo

Methods of treatment involving one or more component of the differentiation medium and/or a pharmaceutical composition of the invention are also provided. In particular, it is envisaged that EECs in vivo can be directed towards particular EEC phenotypes that express particular hormones, and thus the methods of the invention can in some embodiments be used to modulate hormone levels in vivo.

For instance, the inventors have shown that BMP activators promote secretin secretion (and suppress GLP-1 secretion) in EECs (see Example 5). It is therefore envisaged that a BMP activator, or a pharmaceutical composition comprising a BMP activator (with or without other components of the differentiation medium described herein), could be useful in the context of medical uses associated with elevated secretin levels or suppressed GLP-1 levels. Secretin is associated with neutralisation of stomach pH by inhibition of gastric acid secretion (Afroze et al., Ann Transl Med. 2013 October; 1(3): 29) and appetite suppression (Cheng et al., Neuropsychopharmacology. 2011 January; 36(2): 459-471). Increases secretin levels in vivo may therefore be a useful mechanism for treatment of hyperchlorhydria (excess stomach acid) or obesity.

There is therefore provided a method of treating hyperchlorhydria or obesity, wherein the method comprises administering a BMP activator to a subject in need thereof. There is also provided a BMP activator for use in a method of treating hyperchlorhydria or obesity, wherein the method comprises administering a therapeutically effective amount of the BMP activator to a subject in need thereof. There is also provided the use of a BMP activator for the manufacture of a medicament for treating hyperchlorhydria or obesity, wherein the method comprises administering a therapeutically effective amount of the BMP activator to a subject in need thereof. Examples of suitable BMP activators are known in the art and disclosed earlier in this application.

The inventors have also shown that BMP inhibitors promote GLP-1 secretion (and suppress secretin secretion) in EECs (see Example 5). It is therefore envisaged that a BMP inhibitor, or a pharmaceutical composition comprising a BMP inhibitor (with or without other components of the differentiation medium described herein), could be useful in the context of medical uses involving elevated GLP-1 levels or suppressed secretin levels.

For example, GLP-1 (Glucagon-like peptide-1) is an endogenous incretin and plays a significant role in glucose homeostasis (Manandhar & Ahn J Med Chem. 2015 Feb. 12; 58(3): 1020-1037). It binds to and activates the GLP-1 receptor (GLP-1R) belonging to class B family of G-protein-coupled receptors (GPCRs) in order to exert its regulatory functions. Activation of the receptor on the 3-cells leads to a rapid increase in the levels of cAMP and intracellular calcium followed by insulin exocytosis in a glucose-dependent manner. Although GLP-1R in the α-cells is <0.2% of that in the β-cells, GLP-1 inhibits glucagon secretion by 50% through modulation of calcium channel activity. It has been shown that GLP-1 therapy potentiates insulin secretion in both healthy and diabetic patients. Unlike other diabetes drugs, the insulinotropic effect of GLP-1 is self-limiting, as it subsides once the plasma glucose level is lowered to normal range, reducing the risk of hypoglycemia. In addition, GLP-1 regulates postprandial glucose elevation through several other mechanisms, including promoting insulin gene transcription, stimulating pancreatic β-cell proliferation and neogenesis, inhibiting 3-cell apoptosis, and blocking glucagon release. It also prevents gastric emptying and induces satiety, leading to body weight decrease. GLP-1 therapy seems to offer cardioprotective effects as well. However, the endogenous GLP-1 has a very short half-life, owing to rapid metabolic degradation by proteases like dipeptidyl peptidase IV (DPP-IV) and neutral endopeptidase 24.11 (NEP 24.11). This limits its use as a therapeutic. GLP agonists exist (such as DPP-IV inhibitors), which are thought to stabilise GLP-1. A suspected disadvantage of DPP-IV inhibitors is that GLP1 is just stabilized, and not controlled by endogenous food intake. There is therefore a need for alternative means for alternative and improved therapies for treating diabetes mellitus or diseases and disorders associated therewith.

The inventors hypothesised that in vivo administration of a BMP inhibitor could increase GLP-1 secretion in EECs and thus act as a therapy for treating diabetes mellitus and associated diseases and disorders. The inventors showed that administration of a BMP inhibitor to mice increased GLP-1 secretion (see Example 6). Increasing the number of GLP1 cells is advantageous for therapy because these cells still require food intake to release their GLP1. Thus the GLP1 peak will be higher at times when increased levels of insulin are required by the subject. It is also particularly advantageous for patients with low numbers of GLP1 cells.

There is therefore provided a method for treating diabetes mellitus or a disease or disorder associated therewith, wherein the method comprises administering a therapeutically effective amount of a BMP inhibitor to a subject in need thereof. There is also provided a BMP inhibitor for use in a method of treating diabetes mellitus or or a disease or disorder associated therewith, wherein the method comprises administering a therapeutically effective amount of the BMP inhibitor to a subject in need thereof. There is also provided the use of a BMP inhibitor for the manufacture of a medicament for treating diabetes mellitus or a disease or disorder associated therewith, wherein the method comprises administering a therapeutically effective amount of the BMP inhibitor to a subject in need thereof. Examples of suitable BMP inhibitors are known in the art and disclosed earlier in this application.

“Subject” may refer to a human or any non-human animal (such as any mouse, rat, rabbit, dog, cat, cattle, swine, sheep, horse or primate). In preferred embodiments, the subject is a mammal, more preferably a human. A subject can be a patient, which refers to a human presenting to a medical provider for diagnosis or treatment of a disease. A subject can be afflicted with or is susceptible to a disease or disorder but may or may not display symptoms of the disease or disorder.

“Therapeutically effective amount” refers to an amount of a therapeutic agent that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by the skilled person that a therapeutically effective amount is typically administered via a dosing regimen comprising at least one unit dose.

“Treating”, “treat”, “treatment” as used throughout this disclosure refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

“Diabetes mellitus” may be Type I diabetes and Type II diabetes. It may alternatively be gestational diabetes. “Diabetes mellitus” also encompasses patients that are insulin insensitive but that may still be pre-diabetic. “Associated diseases and disorders” include, but are not limited to hyperglycemia, obesity, coeliac disease, thyroid disease, polycystic ovary syndrome, diabetes insipidus, necrobiosis lipoidica diabeticorum, mastopathy, muscular conditions, and dental problems.

For example, the invention provides the following numbered embodiments.

1. A BMP inhibitor for use in a method of treating or preventing diabetes mellitus or an associated disease or disorder, wherein the method comprises administering a therapeutically effective amount of the BMP inhibitor to a subject in need thereof.

2. A BMP inhibitor for use in a method of treating or preventing diabetes mellitus or an associated disease or disorder by increasing GLP-1 secretion from enteroendocrine cells (to increase insulin levels and thus reduce plasma glucose levels), wherein the method comprises administering a therapeutically effective amount of the BMP inhibitor to a subject in need thereof.

3. The BMP inhibitor for use according to embodiment 1 or embodiment 2, wherein the BMP inhibitor is able to
a. disrupt the interaction of a BMP with a BMP receptor;
b. bind to a BMP receptor and inhibit activation of downstream signalling;
c. inhibit phosphorylation of Smad 1, Smad 5 or Smad 8;
d. inhibit translocation of Smad 1, Smad 5 or Smad 8 to the nucleus;
e. inhibit SMAD 1, SMAD 5 or SMAD 8 mediated transcription of target genes; or
f. inhibit expression, folding or secretion of a BMP.

4. The BMP inhibitor for use according to any one of the previous embodiments, wherein the BMP inhibitor inhibits phosphorylation of Smad 1, Smad 5 or Smad 8 and which is a substituted pyrazolo[1,5-a]pyrimidine derivative, e.g. according to Formula I.

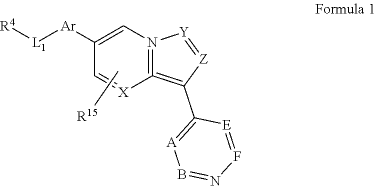

wherein
X and Y are independently selected from CR¹⁵ and N;
Z is selected from CR³ and N;
Ar is selected from substituted or unsubstituted aryl and heteroaryl;
L₁ is absent or selected from substituted or unsubstituted alkyl and heteroalkyl;
A and B, independently for each occurrence, are selected from CR¹⁶ and N;
E and F are both CR⁵ and both occurrences of R⁵ taken together with E and F form a substituted or unsubstituted 5- or 6-membered cycloalkyl, heterocycloalkyl, aryl, or heteroaryl ring;
R³ is selected from Hand substituted or unsubstituted alkyl, cycloalkyl, halogen, acylamino, carbamate, cyano, sulfonyl, sulfoxido, sulfamoyl, or sulfonamido;
R⁴ is selected from Hand substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido;
R¹⁶, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido,
or a pharmaceutically acceptable salt or ester thereof.

5. The BMP inhibitor for use according to embodiment 4, wherein:
a. A and B are each CH;
b. E and F are each CR⁵, and together with the atoms to which both instances of R⁵ are attached form a 6-membered ring;
c. E and F together represent the group:

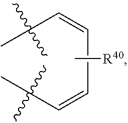

wherein R⁴⁰ is absent or represents from 1-4 substituents selected from substituted or unsubstituted alkyl, cycloalkyl, halogen, acylamino, carbamate, cyano, sulfonyl, sulfoxido, sulfamoyl, or sulfonamido;
d. L₁ has a structure

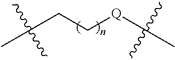

wherein
Q is selected from CR¹⁰R¹¹, NR¹², O, S, S(O), and SO₂; and
R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido;
R¹² selected from H and substituted or unsubstituted alkyl, cycloalkyl, heterocyclyl, heterocyclylalkyl, amino, acylamino, carbamate, amido, amidino, sulfonyl, sulfamoyl, or sulfonamido; and
n is an integer from 0-4;
e. R⁴ is selected from:

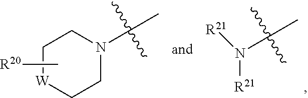

wherein
W is absent or is C(R²¹)₂, O, or NR²¹.
R²⁰ is absent or is selected from substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfoxido, sulfamoyl, and sulfonamido; and
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido; and/or
f. Ar is a 6-membered aryl or heteroaryl ring, optionally, wherein L₁ is disposed on the para-position of Ar relative to the bicyclic core.

6. The BMP inhibitor for use according to embodiments 4 or 5, wherein the therapeutically effective amount is
a. at least 0.1 mg/kg, at least 0.2 mg/kg, at least 0.5 mg/kg, at least 1.0 mg/kg, at least 2 mg/kg, at least 5 mg/kg, at least 10 mg/kg, at least 20 mg/kg, at least 30 mg/kg or about 35 mg/kg;
b. between 0.1 mg/kg and 50 mg/kg, between 0.1 mg/kg and 30 mg/kg, between 0.1 mg/kg and 10 mg/kg, between 0.1 mg/kg and 1 mg/kg, between 1 mg/kg and 50 mg/kg, between 1 mg/kg and 30 mg/kg, between 1 mg/kg and 10 mg/kg; and/or
c. wherein the therapeutically effective amount is administered once, twice or three times daily.

7. The BMP inhibitor for use according to any one of embodiments 1 to 3, wherein the BMP inhibitor is selected from:
a. dorsomorphin or LDN193189 or an analog or variant thereof; and/or
b. noggin, sclerostin, chordin, CTGF, follistatin, gremlin, tsg, sog or an analog or variant thereof.

8. The BMP inhibitor for use according to any one of the previous embodiments, wherein the BMP inhibitor is in the form of a pharmaceutically acceptable salt.

9. The BMP inhibitor for use according to any one of the previous embodiments, wherein the subject is a mammal, preferably a human, cat or dog.

10. The BMP inhibitor for use according to any one of the previous embodiments, wherein the subject is a human.

11. The BMP inhibitor for use according to any one of the previous embodiments, wherein the BMP inhibitor is administered
a. orally, topically or by injection, preferably orally and/or
b. systemically or locally.

12. The BMP inhibitor for use according to any one of the previous embodiments, wherein the BMP inhibitor is administered in combination with one or more additional diabetes therapeutic e.g. a sulfonylurea, a biguanide, a metformin, an alpha-glucosidase inhibitor, a thiazolidinedione, a meglitinide, a dipeptidyl peptidase-4 inhibitor or other incretin mimetic, an amylin analog, or a glycosuric.

13. The BMP inhibitor for use according to any one of the previous embodiments, wherein the BMP inhibitor is administered in combination with a GLP-1 receptor agonist, e.g. selected from: exenatide, liraglutide, taspoglutide, lixisenatide.

14. The BMP inhibitor for use according to any one of the previous embodiments, wherein the BMP inhibitor is administered in combination with insulin or a biologically active analog thereof.

15. The BMP inhibitor for use according to any one of embodiments 12 to 14, wherein the combination is administered as a single composition or as two separate compositions.

16. The BMP inhibitor for use according to any one of embodiments 12 to 15, wherein the combination is administered simultaneously or sequentially.

17. The BMP inhibitor for use according to any one of the previous embodiments, wherein diabetes mellitus is type 1 diabetes, type 2 diabetes, gestational diabetes or insulin insensitivity, preferably type 2 diabetes.

18. The BMP inhibitor for use according to any one of the previous embodiments, wherein the subject has aberrantly low levels of GLP-1.

19. The BMP inhibitor for use according to any one of the previous embodiments, wherein the method treats diabetes by increasing the number of enteroendocrine cells expressing hormones characteristic of the crypt, wherein hormones characteristic of the crypt include GLP-1, neurokinin A and substance P and glucagon.

20. The BMP inhibitor for use according to any one of the previous embodiments, wherein the method results in an increase of circulating/intestinal/pancreatic GLP-1 hormone levels of at least a 10%, 20%, 30%, 40%, 50%, 60%, 70%, 80%, 90% or 100% relative to the prior to administration of the BMP inhibitor in the same patient.

21. The BMP inhibitor for use according to any one of the previous embodiments, wherein the method results in a fasting plasma glucose level in the subject of less than 10.0 mmol/l, less than 9.0 mmol/l, less than 8.0 mmol/l, less than 7.0 mmol/l, less than 6.9 mmol/l, less than 6.8 mmol/l, less than 6.7 mmol/l, less than 65. mmol/l, less than 6.4 mmol/l, less than 6.3 mmol/l, less than 6.2 mmol/l, less than 6.1 mmol/l or less than 6.0 mmol/l.

### Cellular Therapy

Also included within the scope of the invention are methods of treatment of a human or non-human animal patient through cellular therapy. The term “animal” here denotes all mammalian animals. The patient may be at any stage of development, including embryonic and foetal stages. For example, the patient may be an adult, or the therapy may be for pediatric use (e.g. newborn, child or adolescent). Such cellular therapy encompasses the administration of cells or organoids generated according to the invention to a patient through any appropriate means. Specifically, such methods of treatment involve the regeneration of damaged tissue. The term “administration” as used herein refers to well recognized forms of administration, such as intravenous or injection, as well as to administration by transplantation, for example transplantation by surgery, grafting or transplantation of tissue engineered cell populations derived from cells or organoids according to the present invention. In the case of cells, systemic administration to an individual may be possible, for example, by infusion into the superior mesenteric artery, the celiac artery, the subclavian vein via the thoracic duct, infusion into the heart via the superior vena cava, or infusion into the peritoneal cavity with subsequent migration of cells via subdiaphragmatic lymphatics, or directly into intestinal sites via infusion into the intestinal arterial blood supply (e.g. into the superior or inferior mesenteric arteries).

In some embodiments, between 10⁴ and 10¹³ cells per 100 kg person are administered per infusion. Preferably, between about 1-5×10⁴ and 1-5×10⁷ cells may be infused intravenously per 100 kg person. More preferably, between about 1×10⁴ and 10×10⁶ cells may be infused intravenously per 100 kg person. In some embodiments, a single administration of cells or organoids is provided. In other embodiments, multiple administrations are used. Multiple administrations can be provided over an initial treatment regime, for example, of 3-7 consecutive days, and then repeated at other times.

It is also possible to obtain an organoid from one single cell expressing Lgr5 as explained herein. This single cell may have been modified by introduction of a nucleic acid construct as defined herein, for example, to correct a genetic deficiency or mutation. It would also be possible to specifically ablate expression, as desired, for example, using siRNA. Potential polypeptides to be expressed could be any of those that are deficient in metabolic diseases, including, for example, a polypeptide deficiency in metabolic liver disease, such as AAT (alpha antitrypsin). For elucidating physiology, we might also express or inactivate genes implicated in the Wnt, EGF, FGF, BMP or notch pathway.

It will be clear to a skilled person that gene therapy can additionally be used in a method directed at repairing damaged or diseased tissue. Use can, for example, be made of an adenoviral or retroviral gene delivery vehicle to deliver genetic information, like DNA and/or RNA to stem cells. A skilled person can replace or repair particular genes targeted in gene therapy. For example, a normal gene may be inserted into a nonspecific location within the genome to replace a non-functional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment. For example, organoid-derived cells may be genetically modified in culture before transplantation into patients.

Thus, in some embodiments, the organoid or population of epithelial stem cells is for use in medicine, e.g. for treating a disorder, condition or disease and/or for use in regenerative medicine.

In one preferred embodiment, for example, if an organoid is to be used for regenerative medicine, the method may start from epithelial cells or from a tissue fragment in which the cells or tissue fragment are autologous or allogeneic. Some degree of patient matching may still be required to prevent the problems of rejection. Techniques for minimising tissue rejection will be known to those of skill in the art.

In embodiments in which the organoids and/or cells are transplanted into a patient, it can be advantageous to administer the cells in a scaffold. Accordingly, there is provided a scaffold comprising one or more organoids of the invention or cells derived from said organoids. A scaffold provides a two-dimensional or three dimensional network. Suitable synthetic materials for such a scaffold comprise polymers selected from porous solids, nanofibers, and hydrogels such as, for example, peptides including self-assembling peptides, hydrogels composed of polyethylene glycol phosphate, polyethylene glycol fumarate, polyacrylamide, polyhydroxyethyl methacrylate, polycellulose acetate, and/or co-polymers thereof (see, for example, Saha et al. (2007) Curr Opin Chem Biol. 11(4): 381-387; Saha et al. (2008) Biophysical Journal 95:4426-4438; Little et al. (2008) Chem. Rev 108:1787-1796). As is known to a skilled person, the mechanical properties such as, for example, the elasticity of the scaffold influences proliferation, differentiation and migration of stem cells. A preferred scaffold comprises biodegradable (co)polymers that are replaced by natural occurring components after transplantation in a subject, for example to promote tissue regeneration and/or wound healing. It is furthermore preferred that said scaffold does not substantially induce an immunogenic response after transplantation in a subject. Said scaffold is supplemented with natural, semi-synthetic or synthetic ligands, which provide the signals that are required for proliferation and/or differentiation, and/or migration of stem cells. In a preferred embodiment, said ligands comprise defined amino acid fragments. Examples of said synthetic polymers comprise Pluronic® F127 block copolymer surfactant (BASF), and Ethisorb® (Johnson and Johnson). In some embodiments the cells are cultured in the scaffold. In other embodiments, they are cultured and then added to the scaffold.

The uses of the present invention may use a single organoid or they may use more than one organoid, for example, 2, 3, 4, 5, 10, 15, 20, 30, 50, 100, 200 or more organoids. Advantageously, the methods of the present invention allow a great number of organoids and epithelial stem cells to be generated in a short period of time, because they result in exponential growth, thereby ensuring that sufficient cells are available for use in the application of interest. Wherever there is reference herein to a “method of treatment” or “method for treatment”, for example involving the organoids or cells obtained from the organoids of the invention, this also refers equally to organoids or cells “for use in treatment” and to organoids or cells “for use in the manufacture of a medicament”.

### Artificial Organs

In some embodiments, there is provided the use of a differentiated organoid or cells derived from the differentiated organoid in an artificial organ. The artificial organ may be transplanted in vivo by methods explained elsewhere. Alternatively, the artificial organ may be ex vivo. In some embodiments, the ex vivo artificial organ may be connected to a patient, e.g. via the blood supply. For instance, an artificial organ comprising a differentiated organoid may be used as part of a dialysis machine. Thus a differentiated organoid can be used to support a patient with diseased or injured epithelial tissue.

### Uses of Intestinal Organoids and Populations of Cells

As shown in the present Examples, the differentiation media and methods of the invention enhance the differentiation of progenitor cells obtained from the stomach to an enteroendocrine cell fate.

Thus, in some embodiments, the invention provides an intestine organoid or cell obtained from the intestine organoid for use in medicine, e.g. for use in treating a gastric disorder, condition or disease or for use in regenerative medicine.

The invention further provides an intestine organoid or cell obtained from the intestine organoid for use in the treatment of diabetes (such as type I or type II), cystic fibrosis and inflammatory bowel disease (such as Crohn's disease), whereby the treating optionally comprises transplantation of the organoid or cells obtained from the gastric organoid into a patient in need thereof.

### Uses of Gastric Organoids and Populations of Cells

Methods for culturing gastric cells are described in WO 2010/090513. It is envisaged that the differentiation media and methods of the invention will enhance the differentiation of progenitor cells obtained from the stomach to an enteroendocrine cell fate.

Thus, in some embodiments, the invention provides a gastric organoid or cell obtained from the gastric organoid for use in medicine, e.g. for use in treating a gastric disorder, condition or disease or for use in regenerative medicine.

The invention further provides a gastric organoid or cell obtained from the gastric organoids for use in the treatment of gastritis, atrophic gastritis, pyloric stenosis, gastric cancer or peptic ulcers, whereby the treating optionally comprises transplantation of the organoid or cells obtained from the gastric organoid into a patient in need thereof.

### Uses of Pancreas Organoids and Populations of Cells

Methods for culturing pancreatic cells are described in WO 2010/090513. It is envisaged that the differentiation media and methods of the invention will enhance the differentiation of epithelial stem cells obtained from the pancreas to an enteroendocrine cell fate.

Thus, in some embodiments, the invention provides a pancreatic organoid or cell obtained from the pancreatic organoid for use in medicine, e.g. for use in treating a pancreatic disorder, condition or disease or for use in regenerative medicine.

The invention further provides a pancreatic organoid or cell obtained from the pancreatic organoids for use in the treatment of diabetes (e.g. diabetes type I or type II), pancreatitis, pancreatic cancer or cystic fibrosis, whereby the treating optionally comprises transplantation of the organoid or cells obtained from the pancreatic organoid into a patient in need thereof. In some embodiments, the transplanted cells are insulin secreting cells. In other embodiments, the cells are progenitor cells that mature further after transplantation into insulin secreting cells.

### Uses of Lung Organoids and Populations of Cells

Methods for culturing lung cells are described in WO 2016/083613. It is envisaged that the differentiation media and methods of the invention will enhance the differentiation of epithelial stem cells obtained from the lung to an enteroendocrine cell fate.

Thus, in some embodiments, the invention provides a lung organoid or cell obtained from the lung organoid for use in medicine, e.g. for use in treating a lung disorder, condition or disease or for use in regenerative medicine.

The invention further provides a lung organoid or cell obtained from the lung organoids for use in the treatment of small cell lung cancer or non-small cell lung cancer (e.g. adenocarcinoma, squamous cell carcinoma or large cell carcinoma), interstitial lung disease, pneumonia (e.g. organizing pneumonia), tuberculosis, cystic fibrosis, bronchitis, pulmonary fibrosis, sarcoidosis, type II hyperplasia, chronic obstructive pulmonary disease, emphysema, asthma, pulmonary oedema, acute respiratory distress syndrome, wheeze, bronchiectasis, hantavirus pulmonary syndrome, Middle East Respiratory Syndrome (MERS), severe acute respiratory syndrome (SARS) or pneumoconiosis. The invention further provides a lung organoid or cell obtained from the lung organoids for use in the treatment of a pathogenic disease caused by a pathogen such as adenovirus, coronavirus (e.g. SARS-CoV or MERS-CoV), human metapneumovirus, influenza virus, parainfluenza virus, respiratory syncytial virus, rhinovirus, hantavirus, enterovirus (e.g. enterovirus D68 (EV-D68)), Bordetella pertussis, Chlamydophila pneumoniae, Corynebacterium diphtheria, Coxiella burnetii, Haemophilus influenzae, Legionella pneumophila, Moraxella catarrhalis, Mycobacterium tuberculosis, Mycoplasma pneumoniae, Staphylococcus aureus, Streptococcus pneumoniae or Streptococcus pyogenes.

### Quiescent Lgr5+ Stem Cells

Quiescent Lgr5+ stem cells exist in vivo, but had not previously been produced in vitro. The inventors have surprisingly found that inhibition of the EGFR pathway can induce quiescence of Lgr5+ stem cells in vitro.

Accordingly, the invention provides a method for inducing quiescence in an Lgr5+ stem cell, wherein said method comprises:
treating the cell with one or more EGFR pathway inhibitors.

In some embodiments, the method is an in vitro method.

The invention further provides a quiescent stem cell population obtained by a method for inducing Lgr5+ progenitor stem cell quiescence of the invention, wherein the cells express Lgr5 and Lef1 and do not express KI67 and M phase marker phospho-Histone H4.

The invention further provides an in vitro culture comprising a quiescent stem cell population of the invention.

In some embodiments, the quiescent state is maintained for at least 5, at least 6, at least 7, at least 8, at least 9 or at least 10 days. Accordingly, in some embodiments, the quiescent state is maintained for at least 7-10 days.

The quiescent stem cell population of the invention presents multiple advantages and applications. For example, the quiescent stem cell population can be stored (e.g. in a freezer) and it will regrow more efficiently than a non-quiescent stem cell population. In some embodiments, the quiescent stem cell population of the invention is used to study the cell cycle of stem cells or to identify molecules that induce cell cycle entry of stem cells.

### Abbreviations

β-TrCP: β-transducin-repeat-containing protein
BME: Basement membrane extract
Cck: Cholecystokinin
CHGA: Chromogranin A
DAPI: 4′,6-diamidino-2-phenylindole
EdU: 5-ethynyl-2′-deoxyuridine
EEC: enteroendocrine cell
GIP: gastric inhibitory protein
GLP-1: glucagon-like protein 1
GSK-3: glycogen synthase kinase 3
IDMI: IWP2, DAPT and MEK inhibitor
LGR: leucine-rich repeat-containing G protein-coupled receptor
LRP: low-density lipoprotein receptor-related protein
Nts: Neurotensin
PP1: protein phosphatase 1
PP2A: protein phosphatase 2A
PP2C: protein phosphatase 2C
Sct: secretin
Sst: somatostatin

### Definitions

As used herein, the verb “to comprise” and its conjugations is used in its non-limiting sense to mean that items following the word are included, but items not specifically mentioned are not excluded.

In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” or “approximately” means that the value presented can be varied by +/−10%. The value can also be read as the exact value and so the term “about” can be omitted. For example, the term “about 100” encompasses 90-110 and also 100.

All patent and literature references cited in the present specification are hereby incorporated by reference in their entirety.

The following examples are offered for illustrative purposes only, and are not intended to limit the scope of the present invention in any way.

The term “intestine” encompasses colon and small intestine.

### EXAMPLES

### Materials and Methods

### Organoid Culture

The basic culture medium (advanced Dulbecco's modified Eagle's medium/F12 supplemented with penicillin/streptomycin, 10 mM HEPES, Glutamax, B27 [Life Technologies, Carlsbad, CA] and 1 mM N-acetylcysteine [Sigma]) was supplemented with 50 ng/ml murine recombinant epidermal growth factor (EGF; Peprotech, Hamburg, Germany), R-spondin1 (conditioned medium, 5% final volume), and Noggin (conditioned medium, 5% final volume), called “ENR” medium. Conditioned media were produced using HEK293T cells stably transfected with HA-mouse Rspo1-Fc (gift from Calvin Kuo, Stanford University) or after transient transfection with mouse Noggin-Fc expression vector. Advanced Dulbecco's modified Eagle's medium/F12 supplemented with penicillin/streptomycin, and Glutamax was conditioned for 1 week. Cells were plated in BME (Trevigen). For inhibition of EGF signalling, cells were treated with Gefitinib (5 μM; Santa Cruz Biotechnology) and EGF was withdrawn from the medium. Wnt secretion was inhibited with IWP-2 (1.5 μM; Sigma Aldrich) and Notch with DAPT (1 mM, Sigma Aldrich). All treatments were performed on 5 day post passage organoids. For EGFR reactivation experiments, organoids were replated in fresh BME and ENR medium to make sure EGFR inhibitor is washed away. For induction of Cre-ERT activity, organoids were treated O/N with 4-hydroxy tamoxifen (1 uM). All control organoids were treated with similar concentrations of the compound dissolvent, dimethyl sulfoxide (DMSO). During treatments, cells were imaged using an EVOS microscope (Electron Microscopy Sciences).

For the induction of enteroendocrine differentiation, cells were either cultured in ENR or ENR plus Valproic acid and CHIR99021 (Yin et al. (2014) Nature methods 11:106-112). After 5 days of culture, medium was removed and organoids were washed with PBS. The cocktail for EEC differentiation included: IWP2(1.5 μM; Sigma Aldrich), DAPT (1 mM, Sigma Aldrich) and MEK inhibitor PD0325901 (5 μM; Sigma Aldrich).

### Immunostainings

Whole organoids were collected by gently dissolving the matrigel in icecold PBS, and subsequently fixed overnight at 4° C. in 4% paraformaldehyde. Next, organoids were permeabilized and blocked in PBS containing 0.5% Triton X-100 and 2% normal donkey serum (Jackson ImunoResearch) for 30 minutes at room temperature. Organoids were incubated for 2 hours at room temperature in blocking buffer containing primary antibodies. Primary antibodies used were rabbit anti-Lysozyme (1:500; DAKO), goat anti-Chromogranin A (1:500; Santa Cruz), mouse anti-Ki67 (1:250; BD Pharmingen), rabbit anti-phospho-Histone 3 (pH3 Ser10, 1:1000; Millipore), mouse anti-Cytokeratin 20 (1:1000; Dako), goat anti-Cholestocystokin (sc-21617, 1:100; Santa Cruz), rabbit anti-Neurotensin (sc-20806, 1:100; Santa Cruz), goat anti-Secretin (sc-26630,1:100; Santa Cruz), goat anti-Somatostatin (sc-7819, 1:100; Santa Cruz), rabbit anti-Gastric Inhibitory Protein (ab22624-50, 1:500, Abcam), rabbit anti-Glucagon like peptide 1 (ab22625, 1;500, Abcam) and mouse anti-acetylated tubulin (1:100; Santa Cruz). Organoids were incubated with the corresponding secondary antibodies Alexa488, 568 and 647 conjugated anti-rabbit, anti-goat and anti-mouse (1:1000; Molecular Probes), in blocking buffer containing DAPI (1;1000, Invitrogen), or with Phalloidin Texas Red (1:1000; Life technologies). EdU incorporation was visualized using the Click-iT Assay Kit (Thermo Fisher), after 1 hr pre-incubation with EdU (10 uM). LacZ staining was performed as previously described (Barker et al. (2007) Nature 449(7165):1003-7). Sections were embedded in Vectashield (Vector Labs) and imaged using a Sp5 and Sp8 confocal microscope (Leica). Image analysis was performed using ImageJ software.

### FACS Sorting

For FACS analysis of LGR5 and KI67 expression, Lgr5^{GFPDTR} organoids were first dissociated into single cells through mechanical disruption, after 15 minutes of Trypsin treatment at 37° C. (TrypLE Express; Life Technologies, Carlsbad, CA). Single cells were fixed on ice using 4% paraformaldehyde for 30 minutes, and washed 3 times in PBS. Cells were permeabilized in PBS containing 0.5% Triton X-100 for 30 minutes, and were stained with an eFluor-660 conjugated rat anti-KI67 (1:1000; eBioscience) antibody for 30 minutes on ice. For cell cycle analysis, cells were stained in 1 ug/ml Hoechst 33342 (ThermoFisher). Subsequently, stained cells were analyzed on a BD FACS Calibur (BD Biosciences).

For expression analysis, organoids were dissociated and immediately sorted on a BD FACS Aria (BD Biosciences). Cells were sorted as single cells in Trizol in a 96 well plate, or as bulk in Trizol in eppendorf tubes.

### RNA Isolation

For RNA-sequencing, cells were sorted into Trizol (Life Technologies) and total RNA was isolated according to the manufacturer's instructions, with the following alterations. RNA was precipitated overnight at −20° C., with 2 ug glycogen (Life Technologies). No additional RNA isolation step was used for cells sorted into 384-wells. For quantitative PCR analysis, RNA was isolated from organoids using the RNAeasy kit (QIAGEN) as instructed in the manufacturer's protocol.

### Quantitative PCR

PCR analysis was performed using the SYBR-Green and Bio-Rad systems as described (Munoz et al. (2012) The EMBO Journal 31:3079-3091). PCR reactions were performed in triplicate with a standard curve for every primer. Changes in expression was calculated using CFX manager software (Bio-Rad). Primers were designed using the NCBI primer design tool.

| Gene | | |
|---|---|---|
| name | Sense oligo | Antisense oligo |
| Ki67 | CCAGCTGCCTGTAGTGTCAA | TCTTGAGGCTCGCCTTGATG |
| | (SEQ ID NO: 1) | (SEQ ID NO: 2) |
| Ccnb2 | GCCAAGAGCCATGTGACTATC | CAGAGCTGGTACTTTGGTGTTC |
| | (SEQ ID NO: 3) | (SEQ ID NO: 4) |
| Lgr5 | ACCCGCCAGTCTCCTACATC | GCATCTAGGCGCAGGGATTG |
| | (SEQ ID NO: 5) | (SEQ ID NO: 6) |
| Atoh1 | GCTGTGCAAGCTGAAGGG | TCTTGTCGTTGTTGAAGG |
| | (SEQ ID NO: 7) | (SEQ ID NO: 8) |
| ChgA | CAGCTCGTCCACTCTTTCCG | CCTCTCGTCTCCTTGGAGGG |
| | (SEQ ID NO: 9) | (SEQ ID NO: 10) |
| Lyz | GGAATGGATGGCTACCGTGG | CATGCCACCCATGCTCGAAT |
| | (SEQ ID NO: 11) | (SEQ ID NO: 12) |
| Gob5 | ACTAAGGTGGCCTACCTCCAA | GGAGGTGACAGTCAAGGTGAGA |
| | (SEQ ID NO: 13) | (SEQ ID NO: 14) |
| Alpi | AGGATCCATCTGTCCTTTGG | ACGTTGTATGTCTTGGACAG |
| | (SEQ ID NO: 15) | (SEQ ID NO: 16) |
| Sct | GACCCCAAGACACTCAGACG | TTTTCTGTGTCCTGCTCGCT |
| | (SEQ ID NO: 17) | (SEQ ID NO: 18) |
| Glu | CTTCCCAGAAGAAGTCGCCA | GTGACTGGCACGAGATGTTG |
| | (SEQ ID NO: 19) | (SEQ ID NO: 20) |
| Cck | GAAGAGCGGCGTATGTCTGT | CCAGAAGGAGCTTTGCGGA |
| | (SEQ ID NO: 21) | (SEQ ID NO: 22) |
| Sst | GACCTGCGACTAGACTGACC | CCAGTTCCTGTTTCCCGGTG |
| | (SEQ ID NO: 23) | (SEQ ID NO: 24) |
| Gip | AACTGTTGGCTAGGGGACAC | TGATGAAAGTCCCCTCTGCG |
| | (SEQ ID NO: 25) | (SEQ ID NO: 26) |
| Nts | TGCTGACCATCTTCCAGCTC | GAATGTAGGGCCTTCTGGGT |
| | (SEQ ID NO: 27) | (SEQ ID NO: 28) |

### Single Cell and Bulk Sequencing

RNA samples were prepared using a modified version of the CEL-seq protocol as described previously (Grun et al. (2015) Nature 525:251-255; Hashimshony et al. (2012) Cell reports 2:666-673). ERCC spike-in was added to the Trizol solution. RNA pellets were dissolved in primer mix and incubated for 2 minutes at 70° C. Cells sorted into 384-well were directly lysed at 65° C. for 5 minutes. cDNA libraries were sequenced on an Illumina NextSeq500 using 75-bp paired-end sequencing.

Analysis of RNA sequencing data. Paired-end reads were quantified as described before (Grun et al. (2015) Nature 525:251-255) with the following exceptions. Reads that did not align or aligned to multiple locations were discarded. For analysis of the bulk sequencing, UMIs were ignored; instead read counts for each transcript were determined by the number of reads that uniquely mapped to that transcript. This count was divided by the total number of reads that mapped to all transcripts and multiplied by one million to generate the reads-per-million (RPM) count. RPM was used in preference of RPKM because CEL-seq only allows 3′ end sequencing. Differential gene expression was evaluated using the DESeq package in R platform (Anders and Huber (2010) Genome biology 11:R106). Cut-offs used were an adjusted p-value <0.1 and FDR <0.1 and at least 2-fold difference to the compared population. To prevent samples with no reads disabling ratiometric analysis, all 0 reads were converted into 0.1 reads prior to ratio calculation and log 2 conversion. Gene ontology analysis was performed using the Revigo (Supek et al. (2011) PloS one 6:e21800) and Gorilla (Eden et al. (2009) BMC bioinformatics 10:48) softwares. Single cell sequencing data was analyzed as described previously (Grun et al. (2015) Nature 525:251-255).

### Example 1

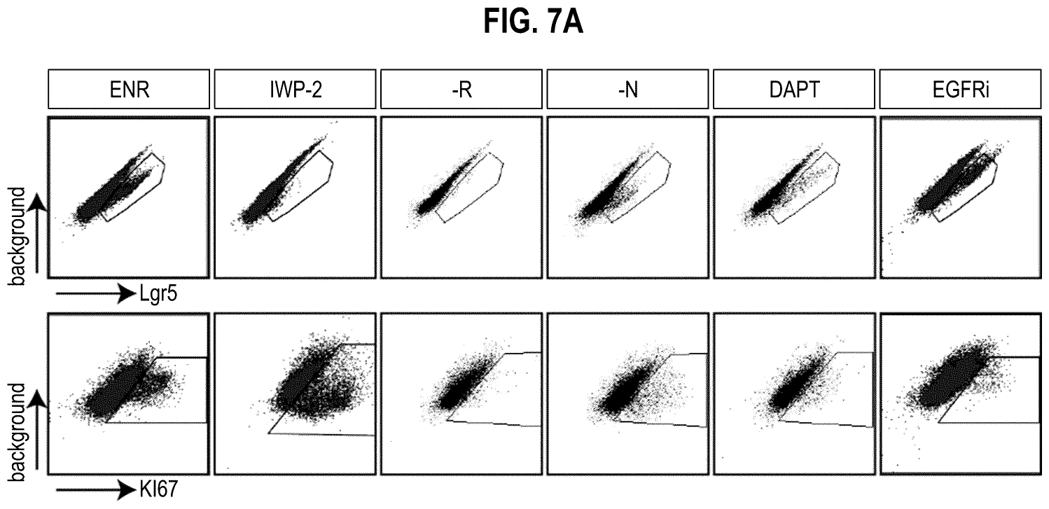

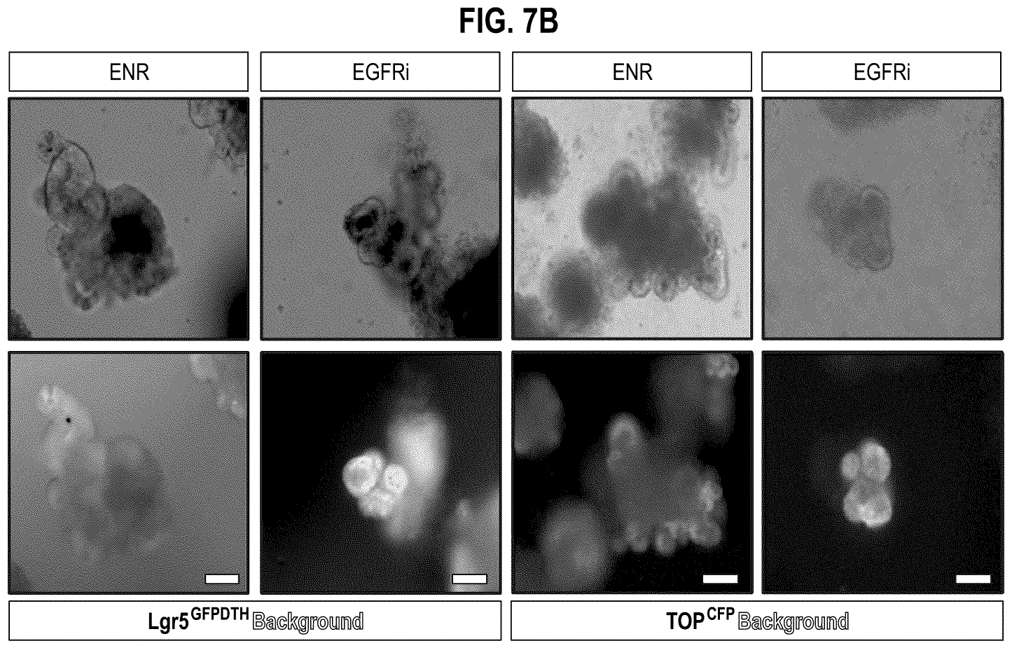

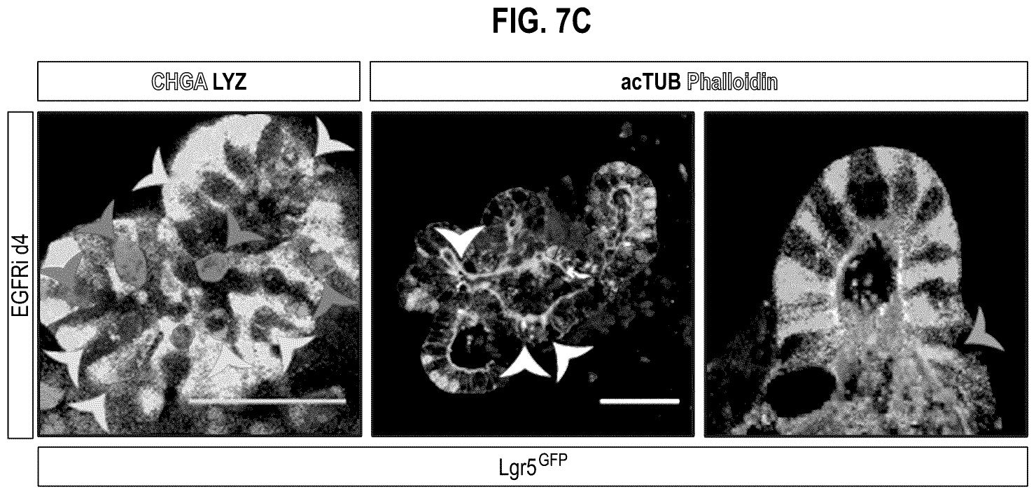

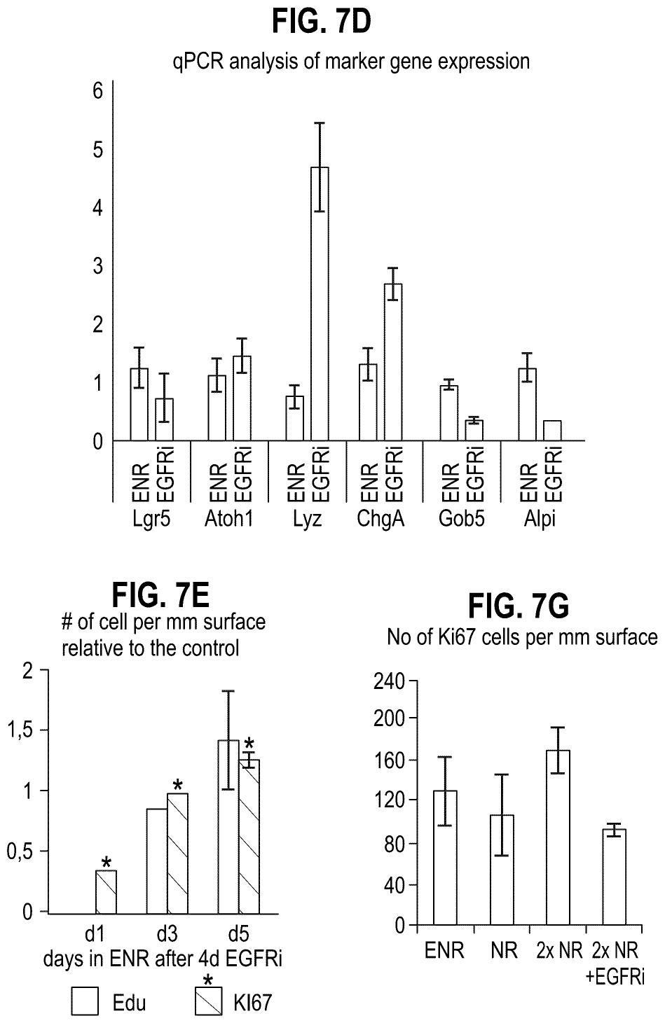

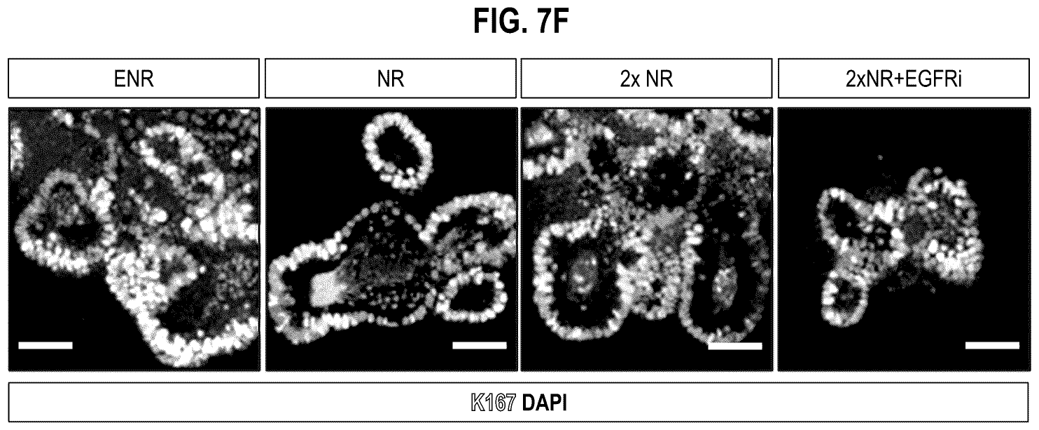

To understand how Lgr5+ stem cells are kept in cycle, we manipulated key signalling pathways active in the crypt niche. Flow cytometry analysis of Lgr5^{GFPDTR} organoids with antibodies against KI67, a marker of cycling cells in all cell cycle phases, confirmed that the majority of the Lgr5+ cells cycle (FIG. 7). We inhibited Wnt signalling using two independent methods; i) IWP-2 treatment inhibits Wnt3 secretion by Paneth cells, and ii) withdrawal of R-spondin1 from the culture medium results in loss of Frizzled receptors from the cell surface. R-spondin1 withdrawal immediately causes loss of Lgr5^{GFP} expression (FIG. 7A). IWP treatment poses a milder Wnt inhibition that depends on dilution of ligands through proliferation. Lgr5^{GFP} expression was gradually down-regulated while stem cells differentiated. Yet, the remaining Lgr5^{GFP} cells maintained KI67 expression (63.5±2.8% versus 94.4±2.1% in control). Withdrawal of the BMP inhibitor Noggin or addition of the Notch inhibitor DAPT both induced a rapid decrease in GFP+ cell numbers, but did not affect proliferation of the remaining Lgr5 cells (82.3±1.4% in Noggin withdrawal, 45.1±10% in DAPT) (FIG. 7). Next, we inhibited EGFR signalling using Gefitinib accompanied by withdrawal of EGF from the culture medium (EGFRi treatment, FIG. 7). While Lgr5^{GFP} expression persisted, the Lgr5 cells eventually lost KI67 expression (13.1±1.0%), indicating an exit from the cell cycle.

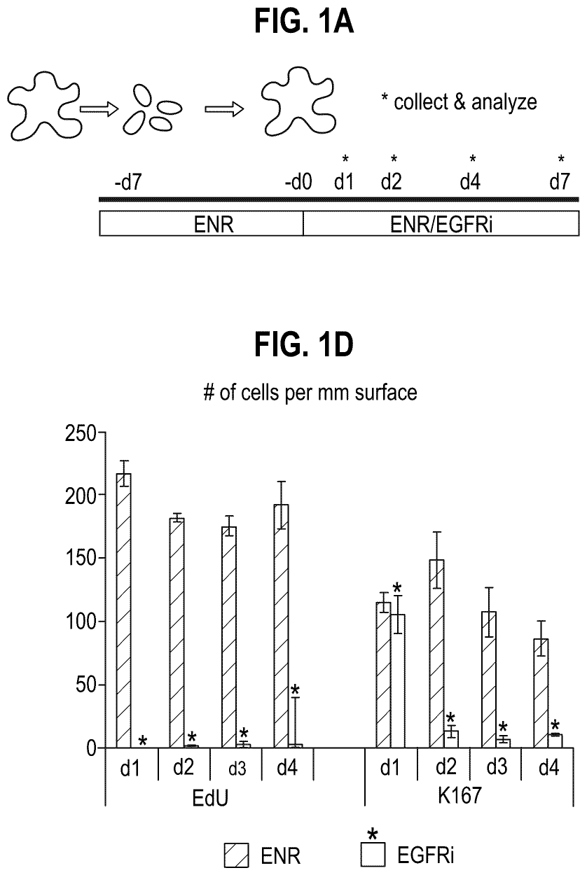

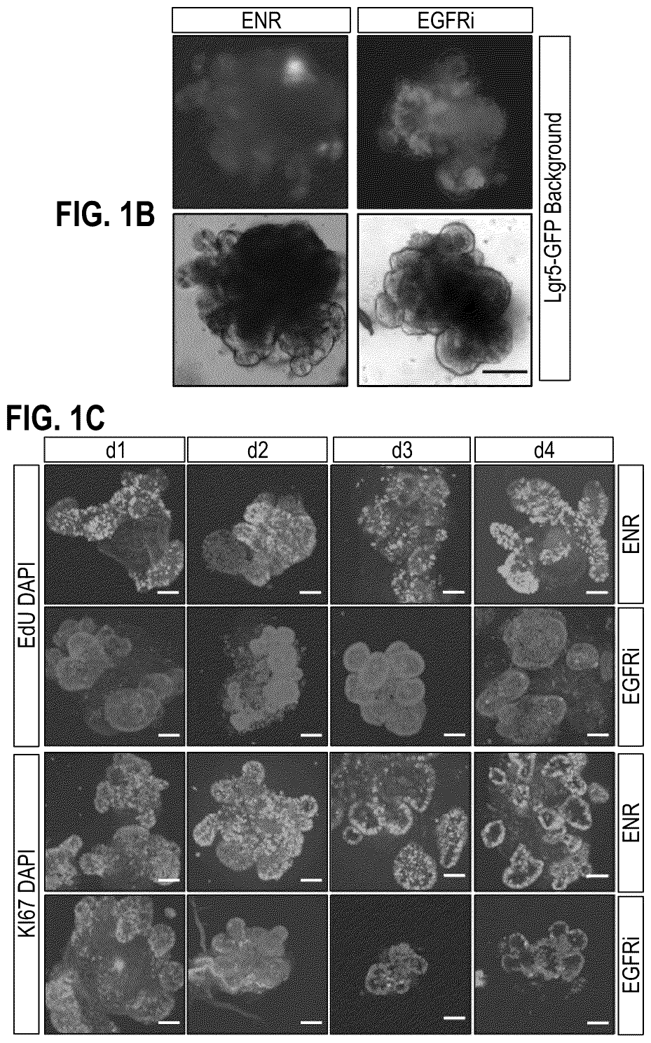

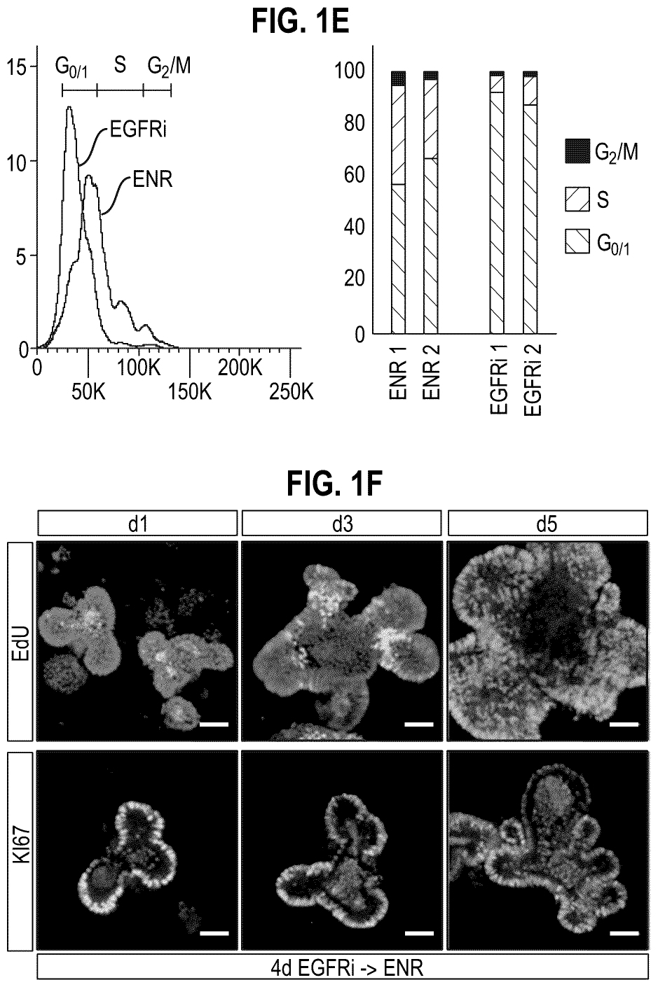

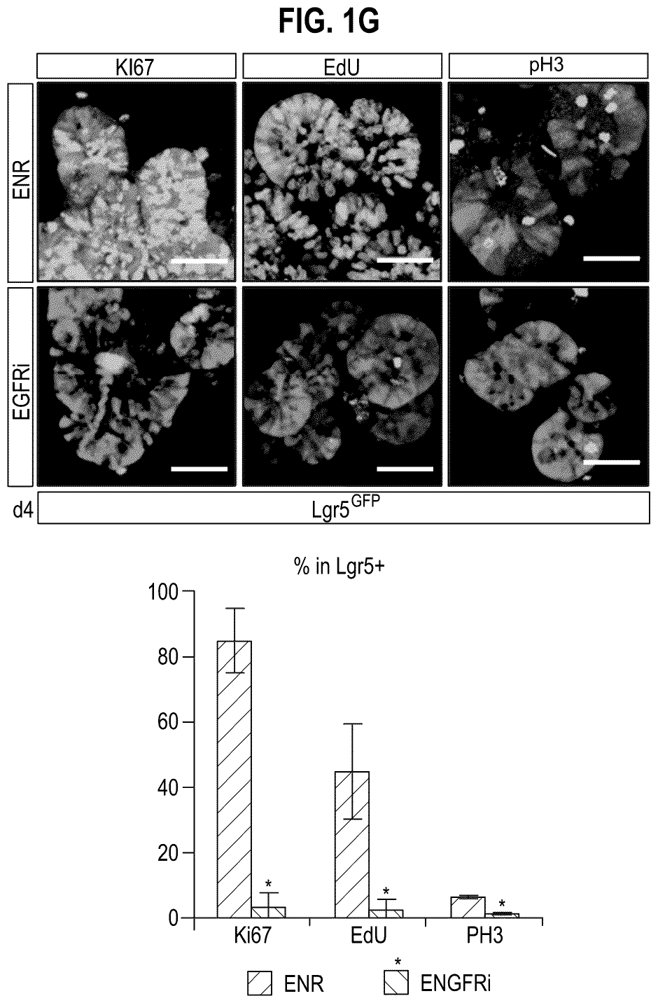

We further analysed the early events associated with EGFR inhibition (FIG. 1A). Despite extensive apoptosis of the ‘villus’ compartments of the organoid, buds resembling crypt structures containing Lgr5+(Lgr5^{GFPiresCreER}) cells persisted for up to a week (FIG. 1). We corroborated these results using the Lgr5^{GFPDTR} allele (Tian et al. (2011) Nature 478:255-259) confirming that CBCs survive in the absence of EGF signalling (FIG. 7). After 4 days of EGFRi treatment, Lgr5+ cells composed 44.4±0.8% (13.6±6.5% in control) of the organoids (FIG. 7). The TCF^{CFP} (Rosa^{TCF-CFP}) Wnt reporter allele (Serup et al. (2012) Disease models & mechanisms 5:956-966) confirmed that Wnt signals remained high in the non-proliferative Lgr5 cells (FIG. 7). The KI67 protein persisted for the first 24 h but was lost from 48 h onwards (FIGS. 1C and 1D). Using a short pulse of EdU as a measure of cells replicating their DNA, we found that EGFRi lead to rapid halt in DNA replication as early as 24 h, which persisted for at least a week (FIGS. 1C and 1D). Consistent with exit from the S phase and eventually from the cell cycle, labelling the DNA content of EGFRi treated organoids using Hoechst DNA staining confirmed that all cells were in G₀/G₁ phase (FIG. 1E). 4 days after EGFRi treatment, reconstitution of EGF signalling induced rapid cell cycle entry within 24 h (KI67+) and progression to the S phase within 48 h (EdU⁺) (FIGS. 1F and 7). Even after a second cycle of EGFRi treatment, quiescent Lgr5 cells re-entered the cell cycle (FIG. 7).

We then further refined the analysis of the non-dividing, EGFRi induced Lgr5+ cells. They lacked the cell cycle marker KI67 and the M phase marker pH3 and did not incorporate EdU, excluding that rare dividing cells persisted during EGFRi treatment (FIG. 1G). Lack of Lysozyme (LYZ) and ChromagraninA (CHGA) implied that the Lgr5+ cells were not differentiated Paneth cells or enteroendocrine cells (EECs), respectively (FIG. 7). Tuft cells (intestinal M-cells) are rare mechanosensory cells also involved in response to parasitic invasion (Howitt et al. (2016) Science 351:1329-1333). They can be distinguished by their typical apical actin bundles, marked by acetylated tubulin and Phalloidin (Hofer and Drenckhahn (1996) Histochemistry and cell biology 105:405-412). The vast majority of the Lgr5+ cells did not display Tuft cell morphology, and—similarly—Tuft cells were mostly Lgr5− (FIG. 7). Quantitative PCR analysis confirmed that the Lgr5 cells did not differentiate into enterocytes, Paneth, EEC, goblet or Tuft cells (FIG. 7).

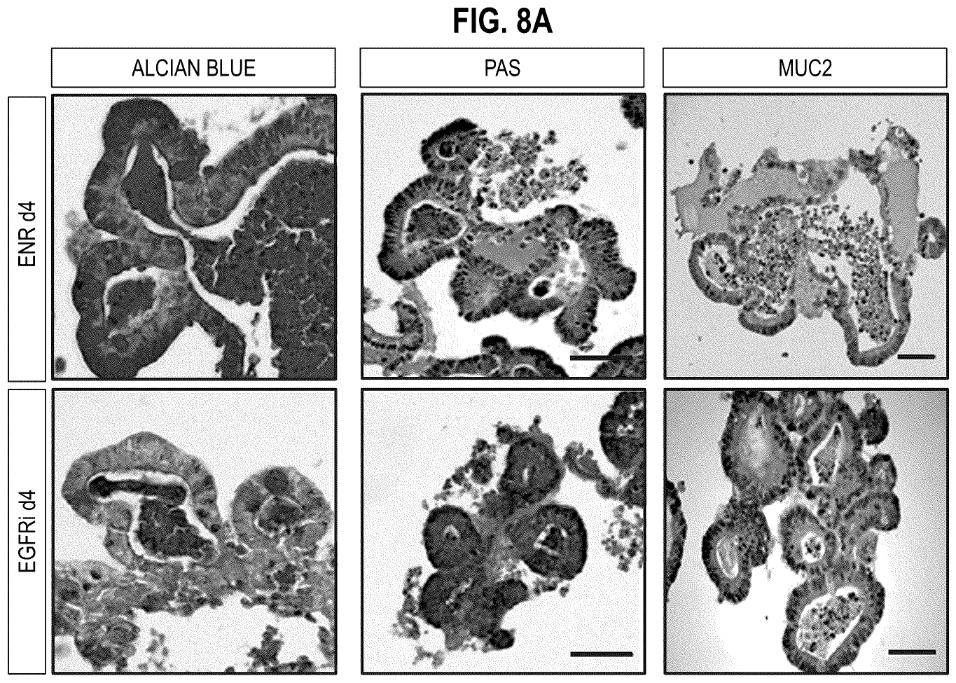

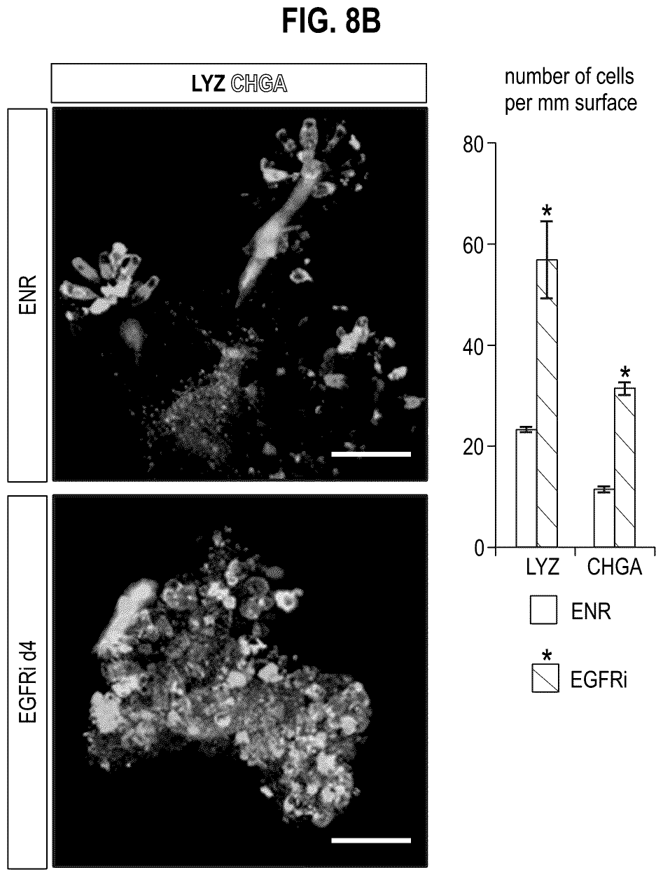

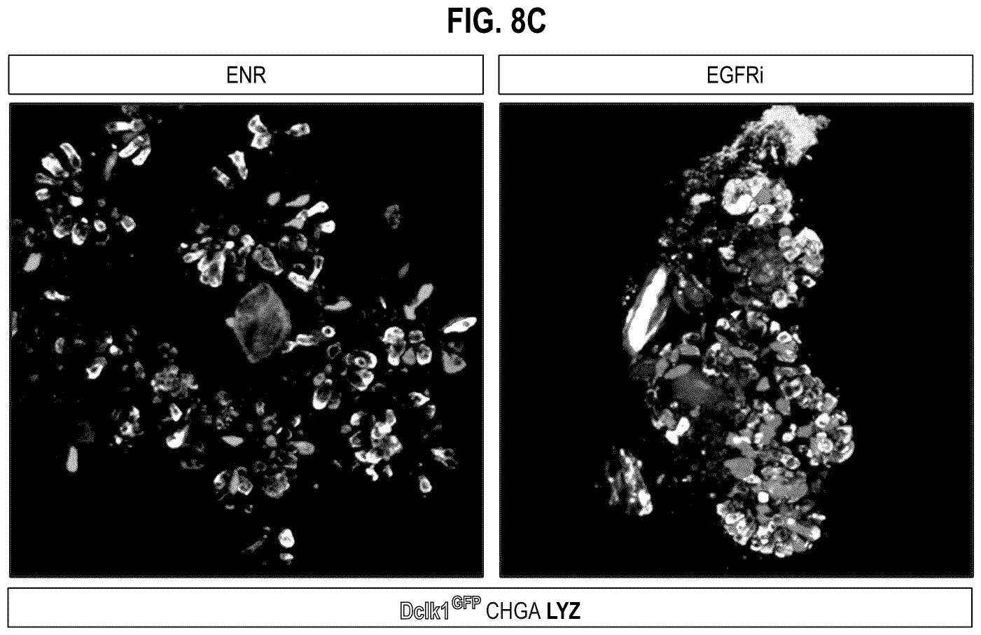

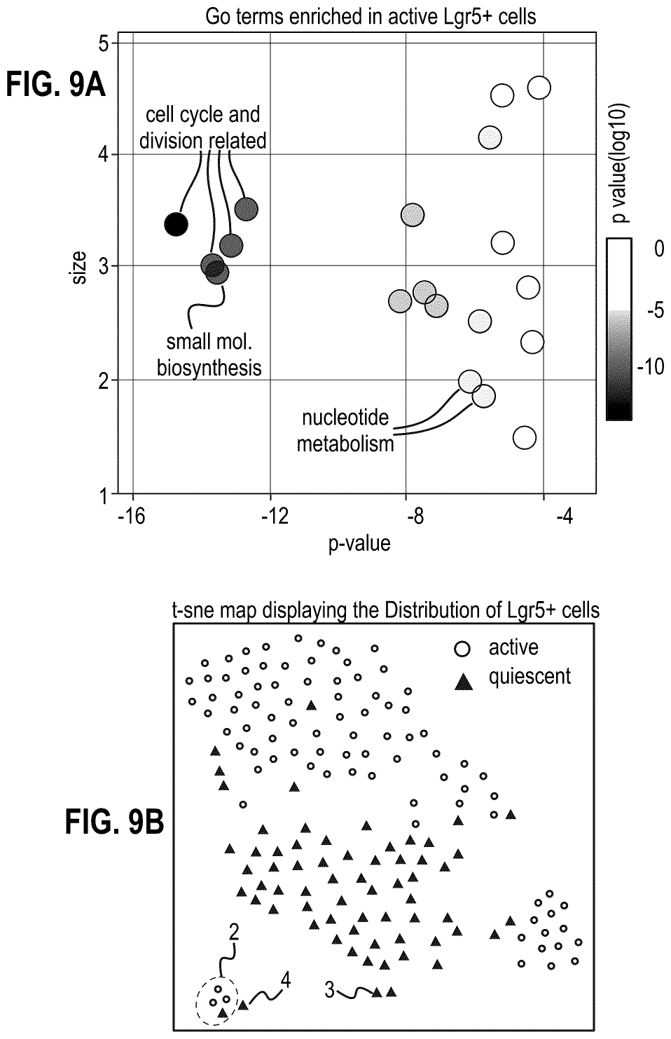

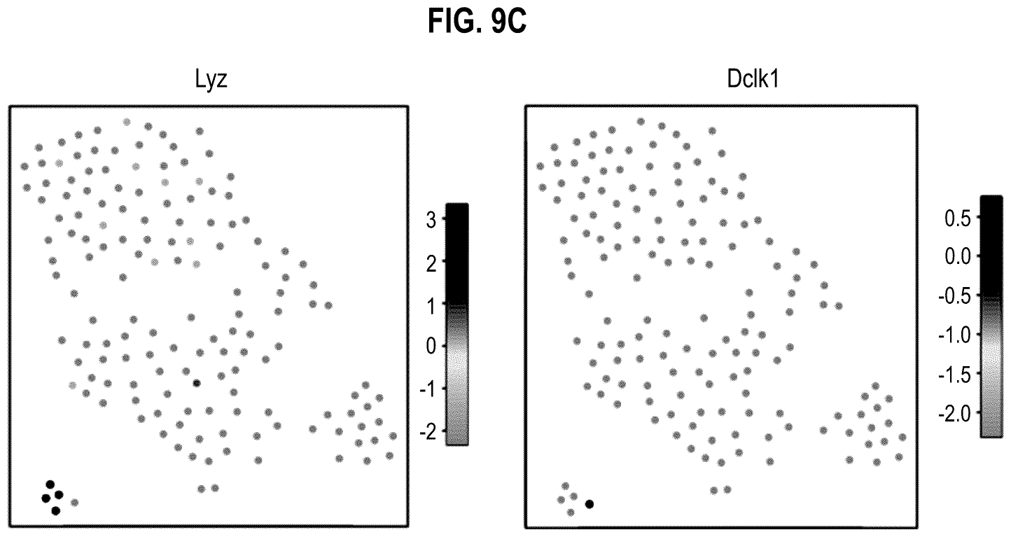

Muc2 (Mucin 2) as well as PAS and Alcian blue staining to visualize mucous structures revealed a significant reduction in the number of goblet cells after EGFRi treatment (Figure S2). The majority of the dividing TA progenitors generate mature enterocytes. While we found an increase in the number of LYZ+ Paneth cells and CHGA+ enteroendocrine cells per bud 4 days after EGFRi treatment, the total number (per organoid) of either cell type was not significantly changed (FIG. 8). The total number of Tuft cells increased upon EGFRi treatment (Figure S2). We corroborated these results using the Dclk1GFPiresCreER allele (Nakanishi et al. (2013), confirming that EGFRi treatment increased the absolute number of Dclk1+ Tuft cells 3.2-fold (11.3±6.6 in ENR, 35.8±8.8 in EGFRi) (FIG. 9). In brief, while EGFR inhibition affects the cell type composition in organoids, it drives Lgr5+ cells into quiescence without inducing differentiation into one of the intestinal lineages.

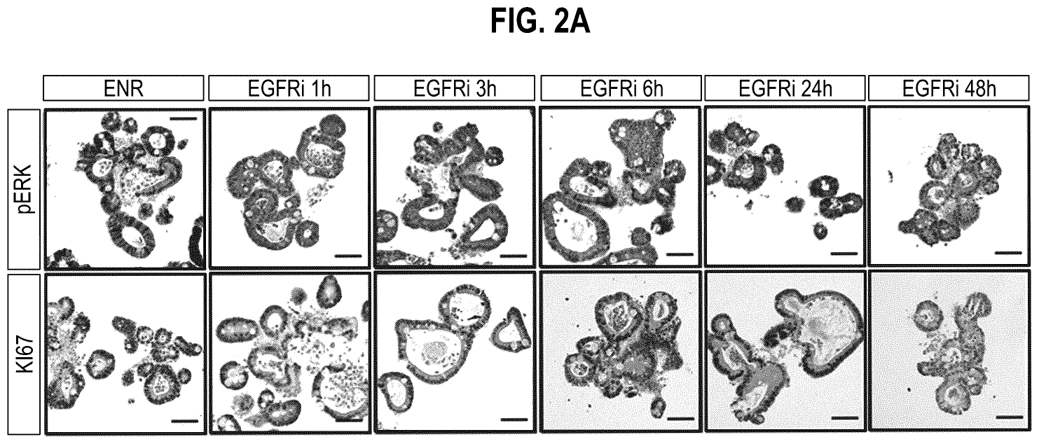

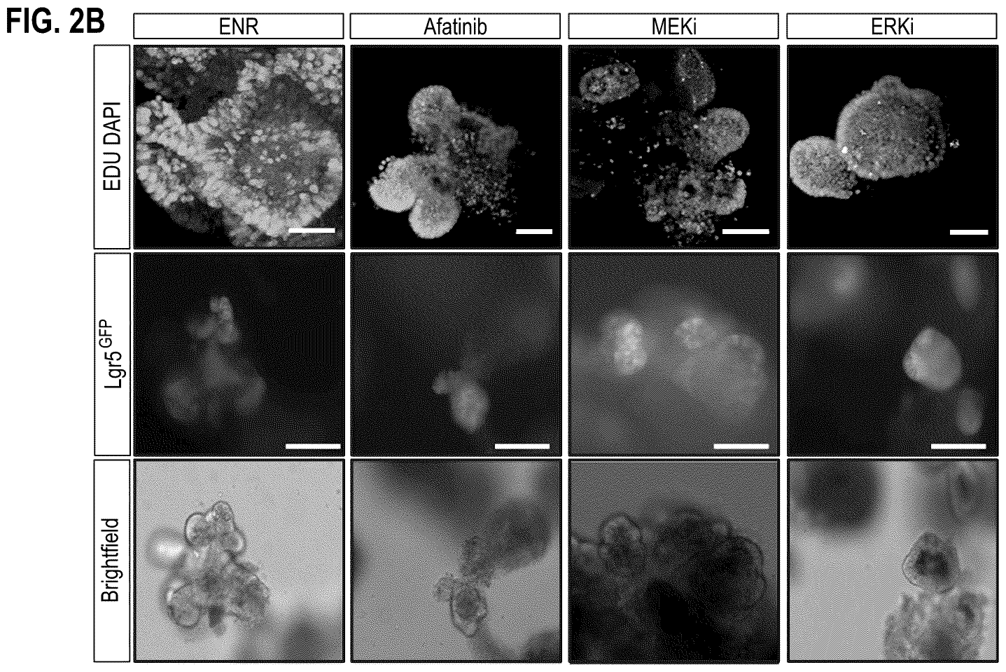

MAPK signalling is a major downstream target of EGFR signalling pathway and regulates cell cycle progression. MAPK kinase (Mek) phosphorylates MAPK (Erk) to induce its nuclear localization and activation. EGFRi treatment reduced ERK phosphorylation as early as after 1 h (FIG. 2A). However, we observed a gradual recovery in phospho-ERK (pERK) levels within 48 h, despite continuing quiescence (FIG. 2A). We asked whether Mek/Erk signalling is essential for cell cycle progression of intestinal stem cells using small inhibitors of either Mek (PD0325901; Meki) or Erk (SCH772984; Erki). Both inhibitors induced quiescence of Lgr5+ cells implying that the ERK pathway downstream of EGFR regulates proliferation of Lgr5+ cells (FIG. 2B). The use of Afatinib, which inhibits both EGFR and ErbB2, yielded similar results (FIG. 2B). We concluded that EGFR inhibition through MAPK signalling is sufficient to induce a reversible quiescent state in intestinal organoid stem cells.

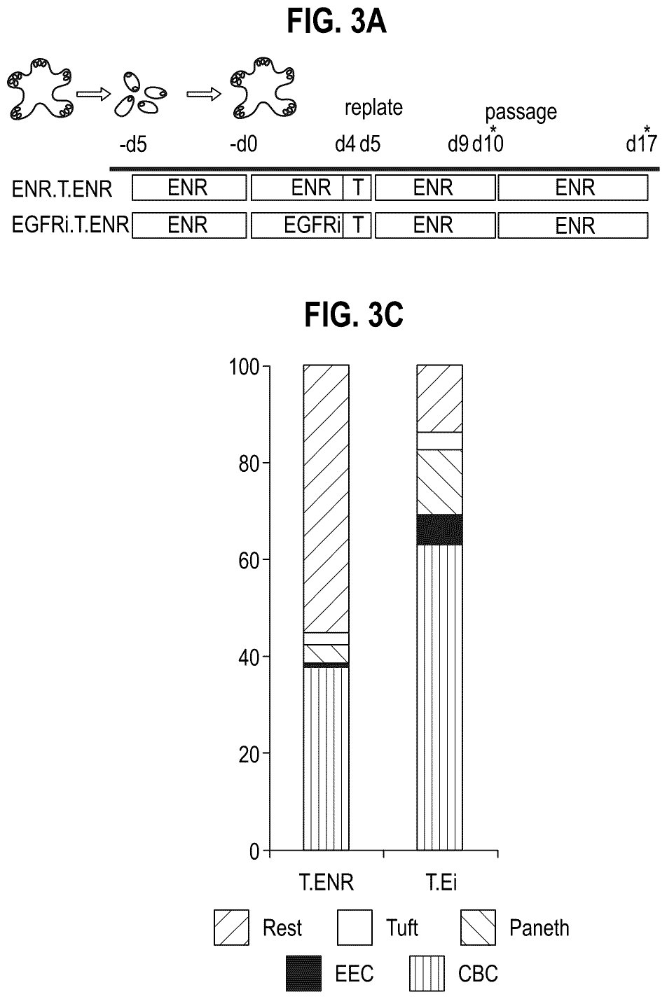

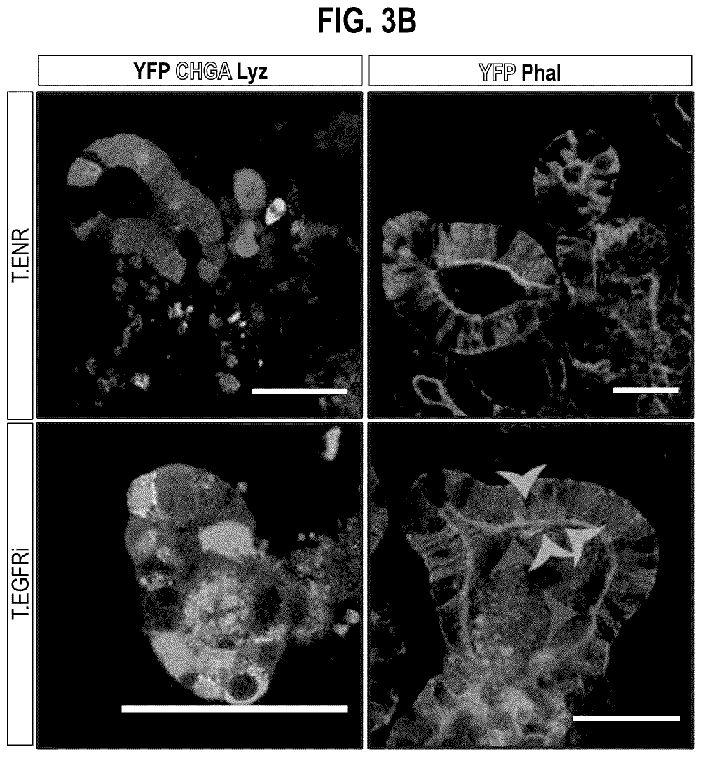

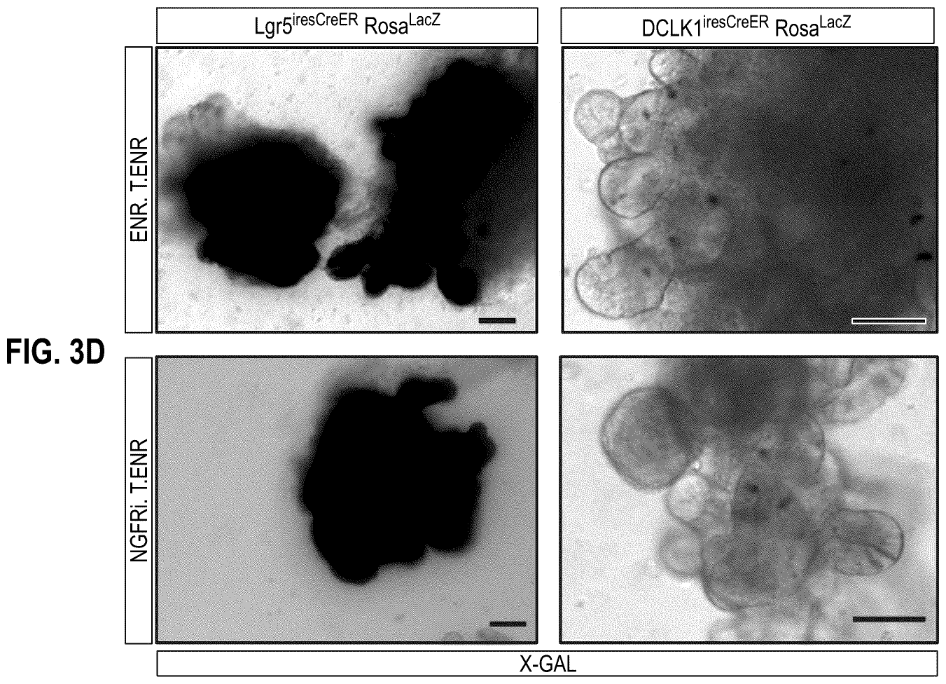

To test whether quiescent Lgr5 cells retain stem cell potential, we used Lgr5^{GFPiresCreER}Rosa26^{YFP/LacZ} and Lgr5^{GFPiresCreER}Rosa26^{tdTomato} organoids to follow the fate of Lgr5+ cells (FIG. 3A). CreER induction using 4-OH Tamoxifen (Tmx) led to rapid recombination that could be visualized by YFP (or tdTomato) fluorescence or LacZ expression (FIGS. 3B and 3C). Labelled, dividing Lgr5 cells generated quiescent Lgr5 cells upon EGFRi treatment, indicating that the latter indeed derived from active Lgr5 cells (FIG. 3B). Moreover, the fraction of labelled Paneth cells and EECs increased upon EGFRi treatment (FIGS. 3B and 3C). In non-EGFRi treated controls, recombined cells generated entire organoids upon passage, indicating efficient labelling of stem cells (FIG. 3D). Upon passage and reactivation of EGF signalling, the progeny of recombined quiescent Lgr5 cells persisted over several passages and generated organoids (FIG. 3D). These results indicated that quiescent Lgr5 cells induced upon EGFRi treatment behave as genuine stem cells.

Tuft cells are non-dividing and might display stem cell-like properties by contributing to tissue regeneration upon injury and tumour growth (Nakanishi et al. (2013) Nature Genetics 45:98-103). We followed the fate of Tuft cells using the organoids derived from the Dclk1^{GFPiresCreER}Rosa^{YFP/LacZ} mouse model. Both in normal and EGFRi treated organoids, labelled cells remained as single cells over time (FIG. 3C). When passaged in the presence of EGF, labelled cells were lost and did not contribute to organoid generation (FIG. 3C) arguing against Dclk1⁺ Tuft cells having stem cell potential in an organoid setting.

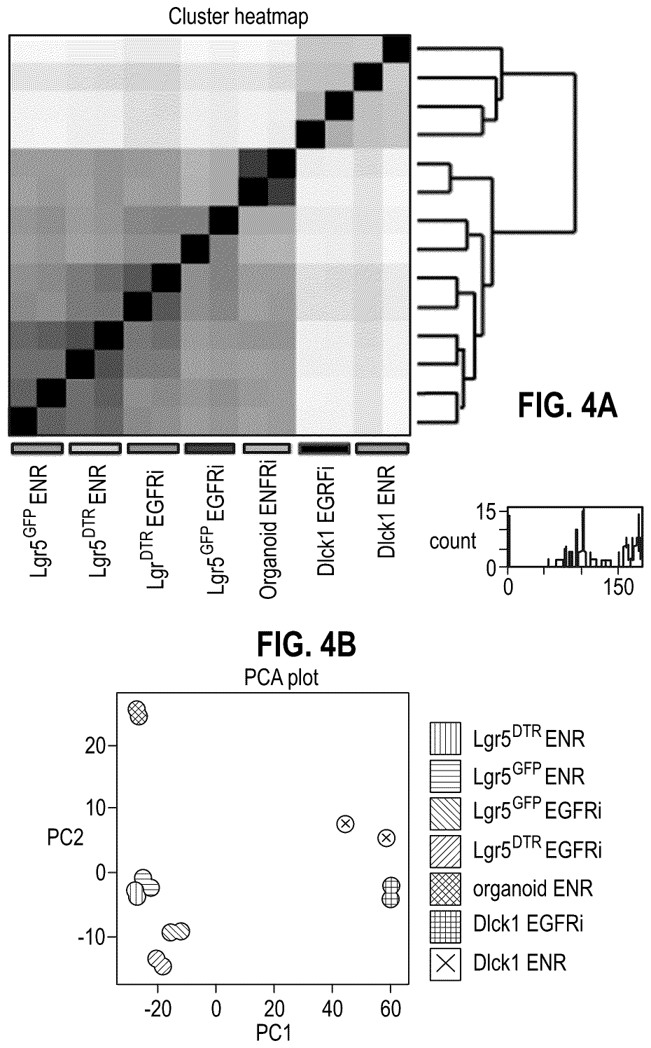

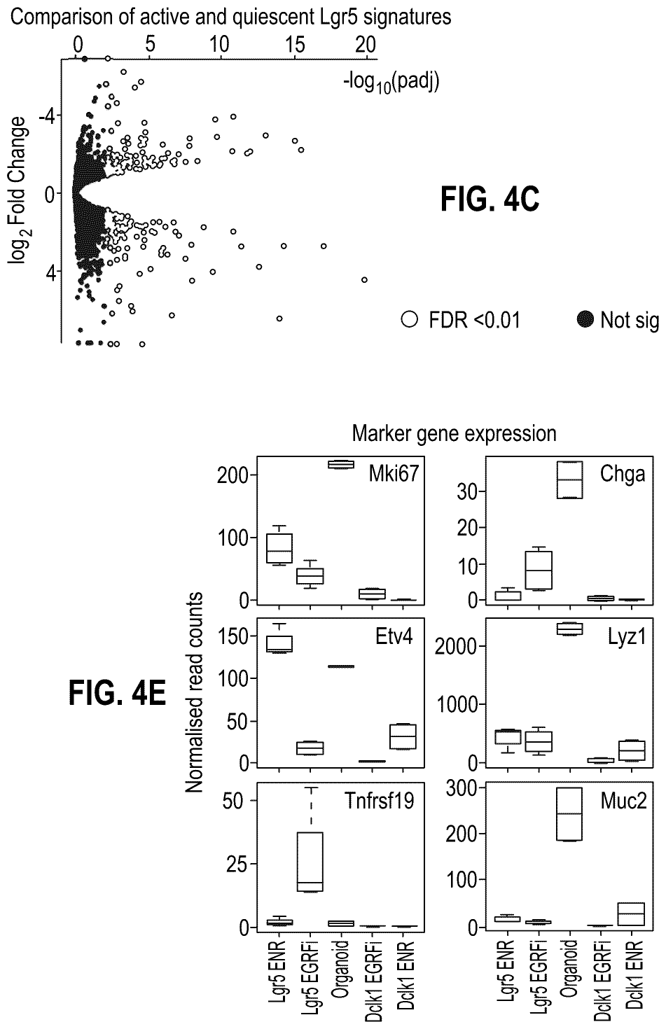

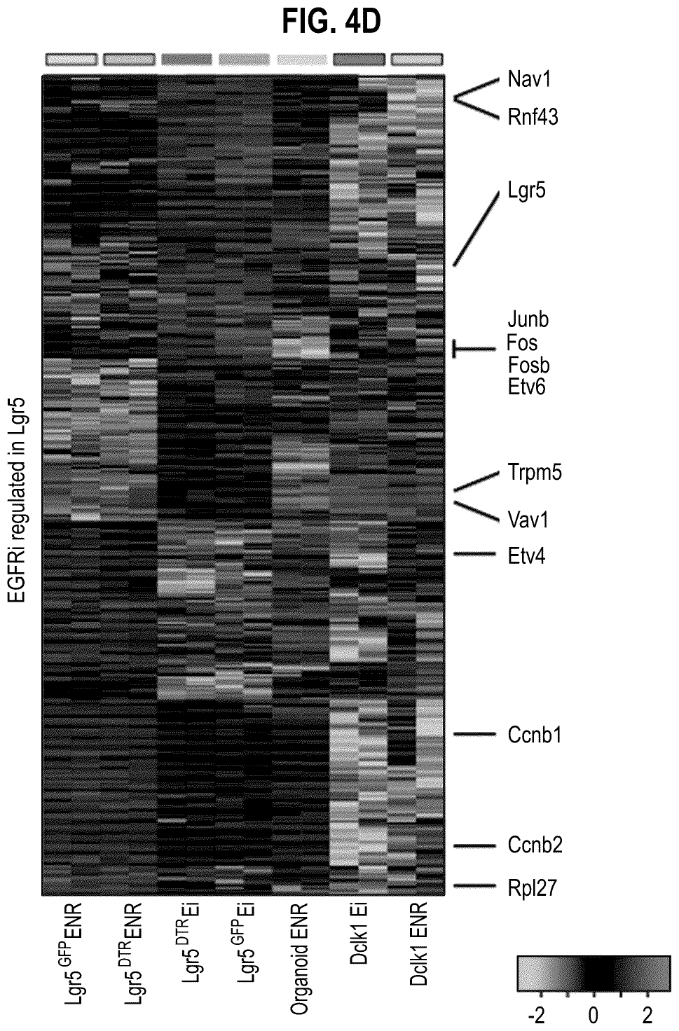

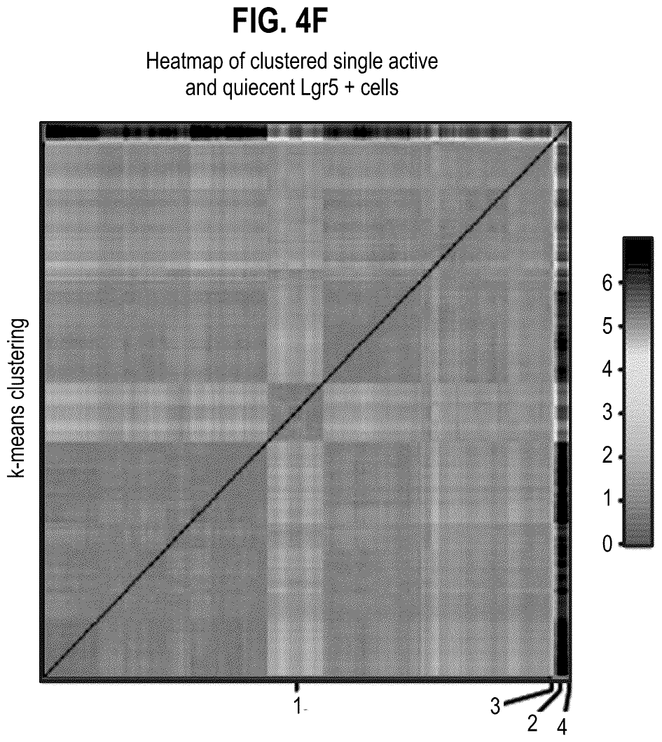

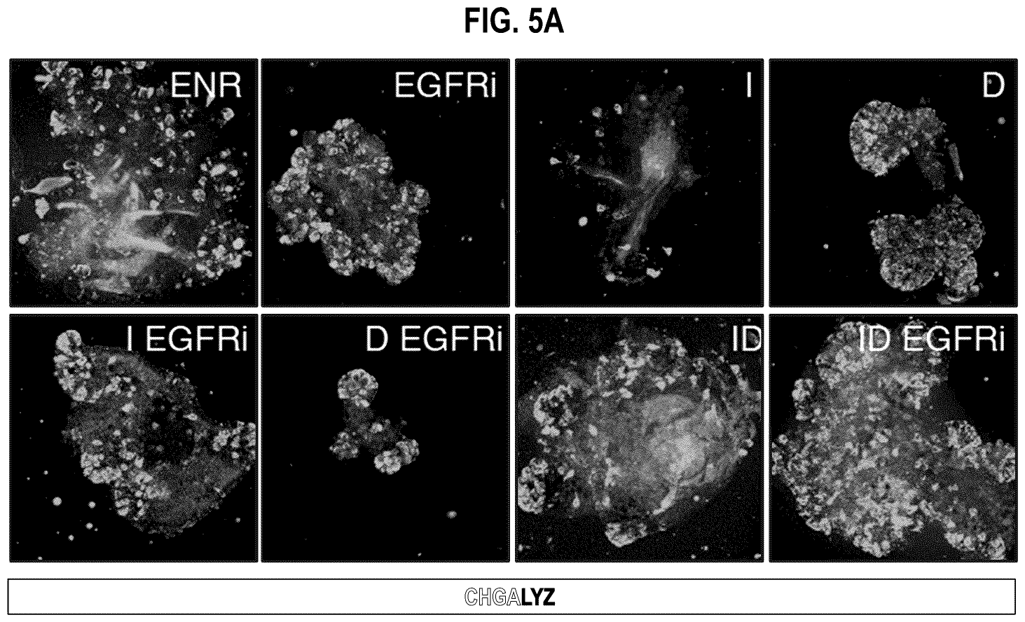

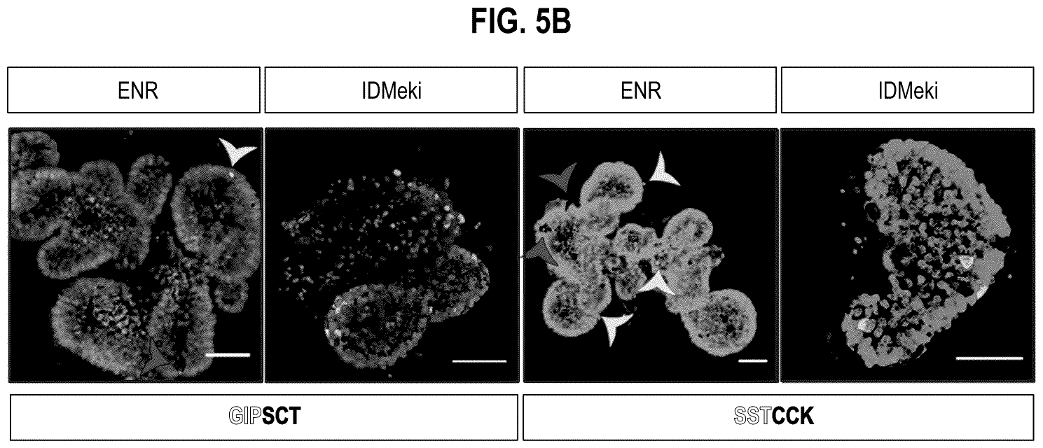

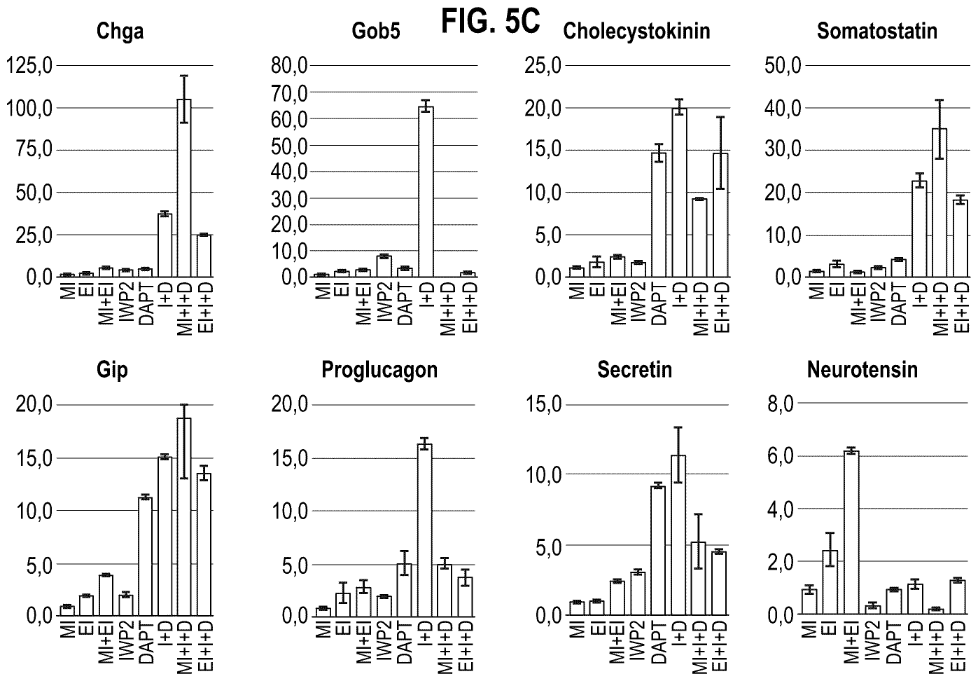

To better understand the molecular characteristics of quiescent Lgr5 cells, we performed RNA sequencing on FACS-isolated active (DMSO control) and quiescent (EGFRi treatment, d4) Lgr5+ stem cells. We included both Lgr5^{GFPiresCreER} (n=2) and Lgr5^{GFPDTR} (n=2) alleles in our study. We also included sorted Dclk1^{GFP} Tuft cells and bulk organoids for comparison. Hierarchical clustering and principal component analysis (PCA) revealed that Lgr5+ cells were more similar to each other than to whole organoids or to Tuft cells (FIGS. 4A and 4B). Consistent with differences in their cell cycle, active and quiescent Lgr5+ stem cells clustered separately (FIGS. 4A and 4B). Differential gene expression analysis between active and quiescent Lgr5 cells revealed 533 differentially regulated genes, 290 of which were enriched in quiescent Lgr5 cells (FDR <0.01 FIG. 5C). Transcriptional effectors of the ERK pathway (Etv4 [7.7×, p-adj<0.001] and Etv5 [7.7×, p-adj<0.001]) were down-regulated in quiescent Lgr5+ stem cells confirming efficient Erk inhibition (FIGS. 4C and 4D). Similarly, several cell cycle associated genes, such as Ccnb1 (2.1×, p-adj<0.005) and Ccnb2 (1.9×, p-adj<0.05) were decreased, consistent with the GO arrest (FIGS. 4C and S9). GO analysis of genes downregulated upon EGFRi treatment confirmed a clear loss of cell cycle-associated genes (Figure S9). One of the defining transcripts in quiescent Lgr5 cells is Lef1 (not detected in active Lgr5, p-adj<0,001), a component of the Wnt signalling pathway. Consistent with our reporter expression, we observed a significant increase in some of the well-known Wnt target genes, including Rnf43 (2.3×, p-adj<0.005) and Lgr5 (2×, padj<0.05) (FIG. 4D). Quantitative PCR analysis in independent experiments confirmed these results (FIG. 9). We also noticed a strong increase of members of the AP-1 family of transcription factors (Junb, Fos, Fosb) in quiescent Lgr5 cells (FIG. 4D). Consistent with the histological analysis, Paneth cell, enterocyte and Goblet cell specific gene expression remained unchanged. Chga, expressed by EECs and their precursors, was 7.3-fold higher in quiescent compared to active Lgr5+ stem cells although high variation at low expression levels impeded further conclusions (p-val=0.019 and padj=0.28). Similarly, while Dclk1 (6×, p-adj<0.05) and some other Tuft cell markers increased upon EGFRi treatment, their levels were significantly lower in quiescent Lgr5 cells than in Tuft cells (FIG. 4D). Global changes in gene expression might be either shared by all quiescent Lgr5 stem cells, or be the result of changes in a specific subpopulation. We have recently shown that individual active Lgr5 cells are rather homogenous (Grun et al. (2015) Nature 525:251-255). We performed single cell sequencing analysis of a total of 192 FACS purified single active or quiescent Lgr5 cells from control or EGFRi treated organoids. Using RaceID (Grun et al. (2015) Nature 525:251-255), we identified a single prominent population (cluster 1) containing cells from both control and EGFRi treated cells (FIG. 4F) containing both active and quiescent Lgr5+ cells (FIG. 9). Minor clusters 2 (four cells) and 4 (one cell) expressed Paneth (Lyz1) and Tuft (Dclk1) cell related genes, while cluster 3 (one cell) resembled cluster 1 (FIG. 9). Thus, the quiescent Lgr5 population is homogenous and despite some significant changes in gene expression, closely resembles quiescent Lgr5 cells.

### Example 2

Quiescence combined with increased Chga and Lgr5 expression (as described in Example 1) is reminiscent of the label-retaining secretory precursors described by Winton and colleagues (Buczacki et al. (2013) Nature 495:65-69). These cells efficiently differentiate into EECs, suggesting that cell cycle exit of Lgr5 cells may facilitate generation of EECs. Hormones expressed by EECs regulate a wide variety of physiological responses, like gastric emptying, release of pancreatic enzymes, mood and glucose tolerance. They are also used to define subtypes (see introduction). G protein-coupled taste receptors have been identified as regulators of hormone secretion (Janssen and Depoortere (2013) TEM 24:92-100). EECs can have direct luminal contact and sense the content with microvilli. Other EECs, the so-called closed-type cells, do not have a luminal lining and need other sources to be stimulated (Janssen and Depoortere (2013) TEM 24:92-100). The length of their basal process also varies and may form synaptic contacts with enteric neurons to connect to the nervous system.

To establish a protocol for EEC differentiation, we used the quiescent Lgr5 cells as a starting point and modulated the Notch and Wnt signalling pathways, which are involved in secretory differentiation. Inhibition of Notch signalling by DAPT treatment (D) enhanced secretory differentiation, leading to a large increase in the number of LYZ+ Paneth cells (FIG. 5A). Inhibition of Wnt secretion using IWP-2 (I) in combination with DAPT abolished Paneth cell differentiation and induced EECs and Goblet cells (FIG. 5A). EGFRi treatment inhibited Goblet cell differentiation, but spared Paneth cells and EECs (FIG. 5A). Combined inhibition of EGFR/WNT/Notch pathways (IDEGFRi) resulted in a massive increase in EECs, while inhibiting enterocyte, Goblet and Paneth cell differentiation (FIG. 5A). Similarly, inhibition of Mek together with Wnt and Notch signalling pathways (IDMeki) increased CHGA+ cell numbers. The different EEC subtypes are rare in normal intestinal organoid cultures (FIG. 5B). IDMeki treatment resulted in a robust increase in the number of these EEC cell types. We used qPCR to corroborate these results. Expression of pan-EEC marker Chga was 25-fold higher in IDEGFRi and over 100-fold higher in IDMeki treated organoids (FIG. 5C). Coincidentally, expression of Sst (55×), Gip (14×), Sct (5×), Cholecystokinin (15×) and Glucagon (Gcg/Proglucagon, 4×) mRNA were upregulated upon IDEGFRi treatment, following a similar trend to IDMeki (FIG. 5C). Nts was the sole hormone analyzed that was expressed at control levels. Thus, our protocol successfully generated high numbers of EECs and various subtypes expressing a panel of hormones regulating mammalian physiology (Egerod et al. (2012) Endocrinology 153:5782-5795).

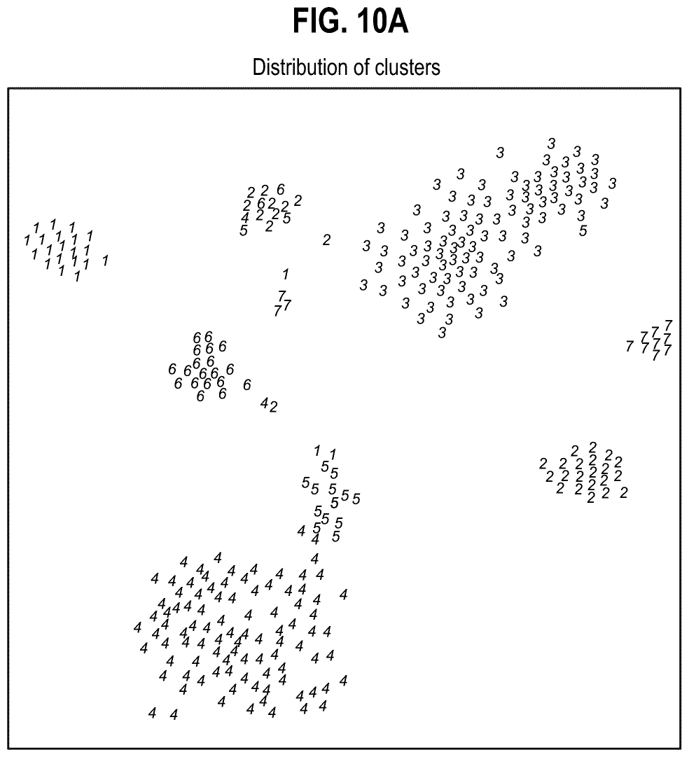

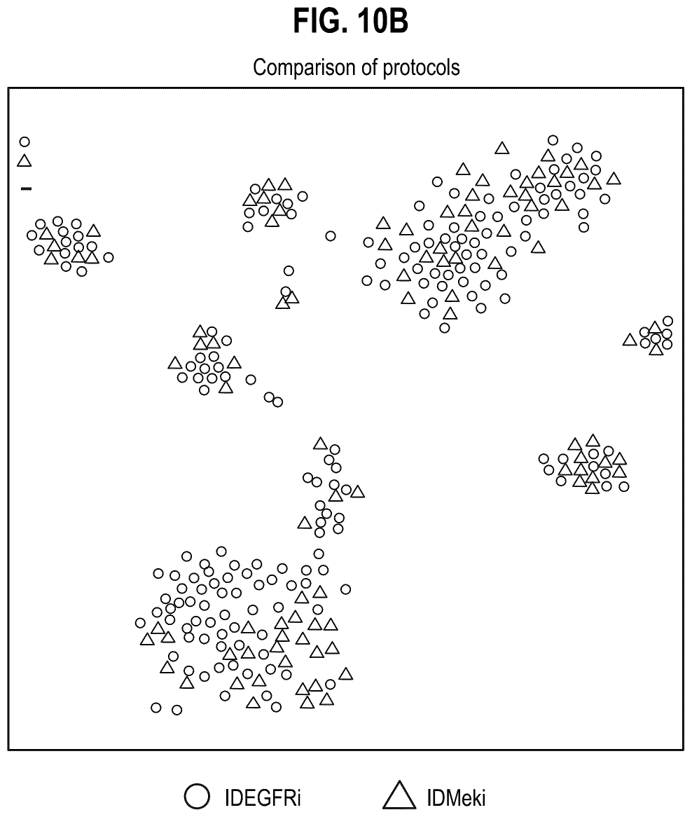

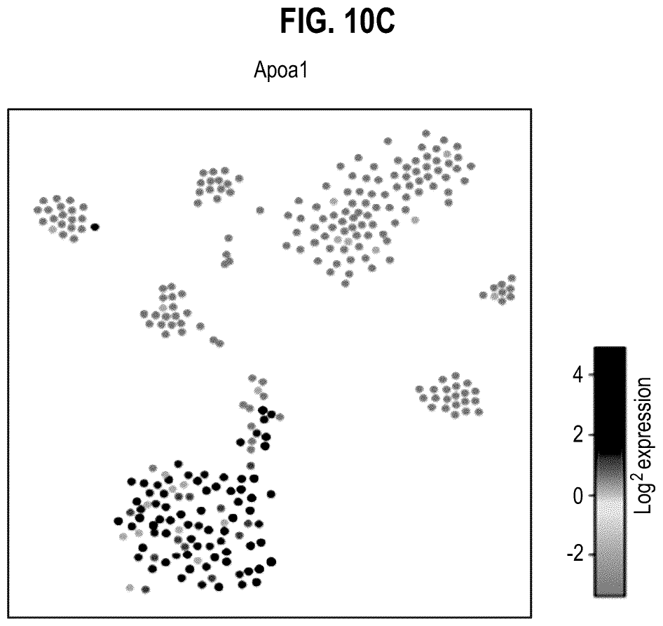

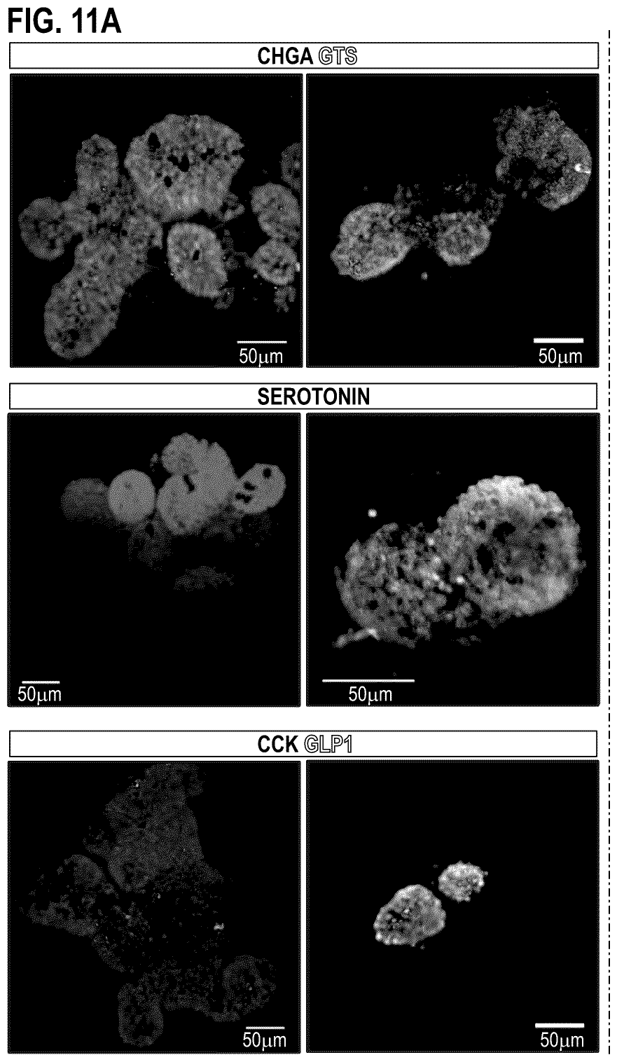

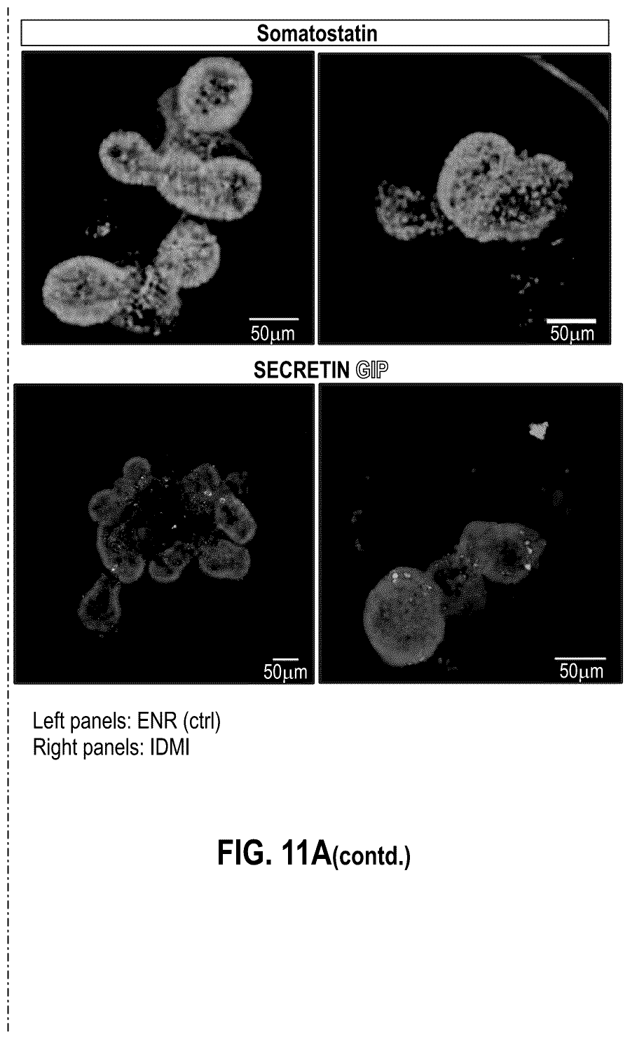

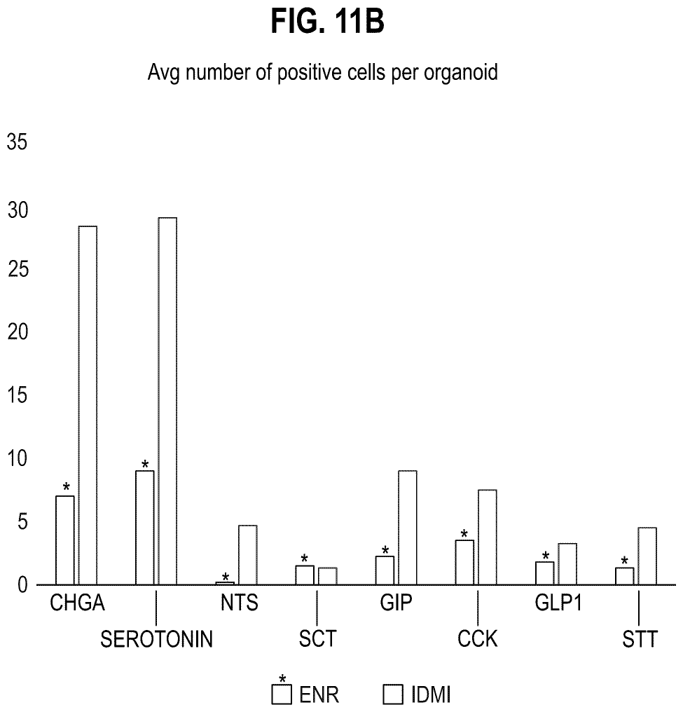

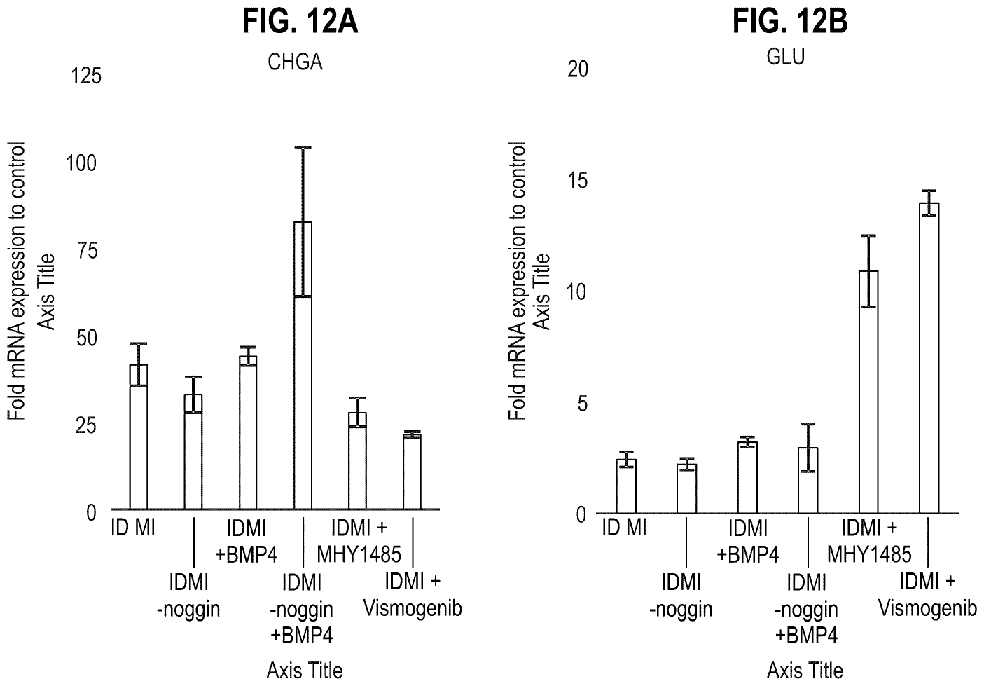

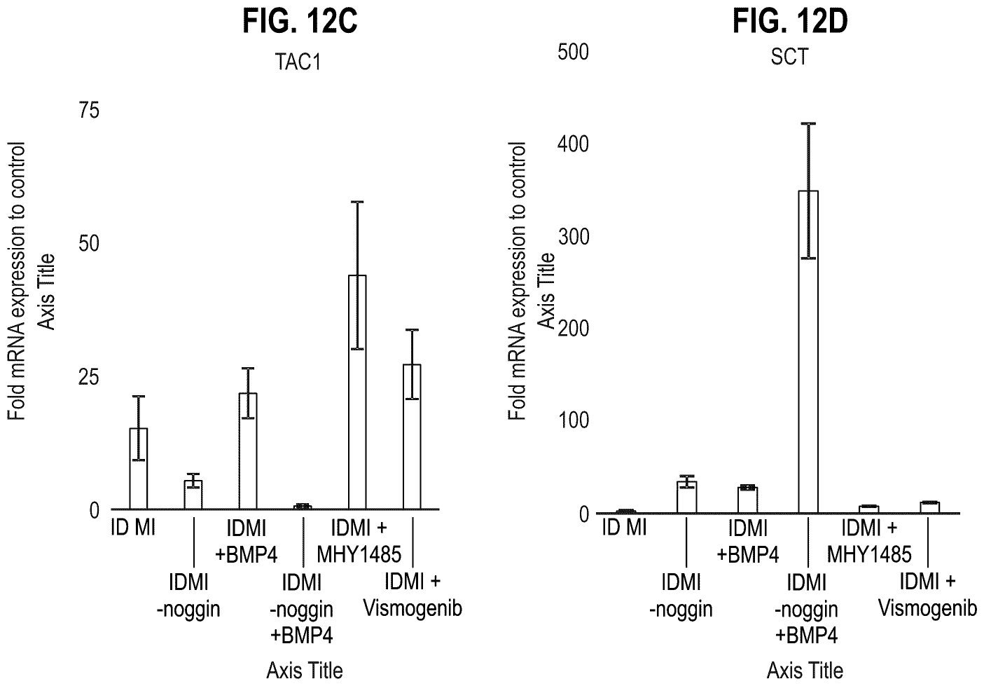

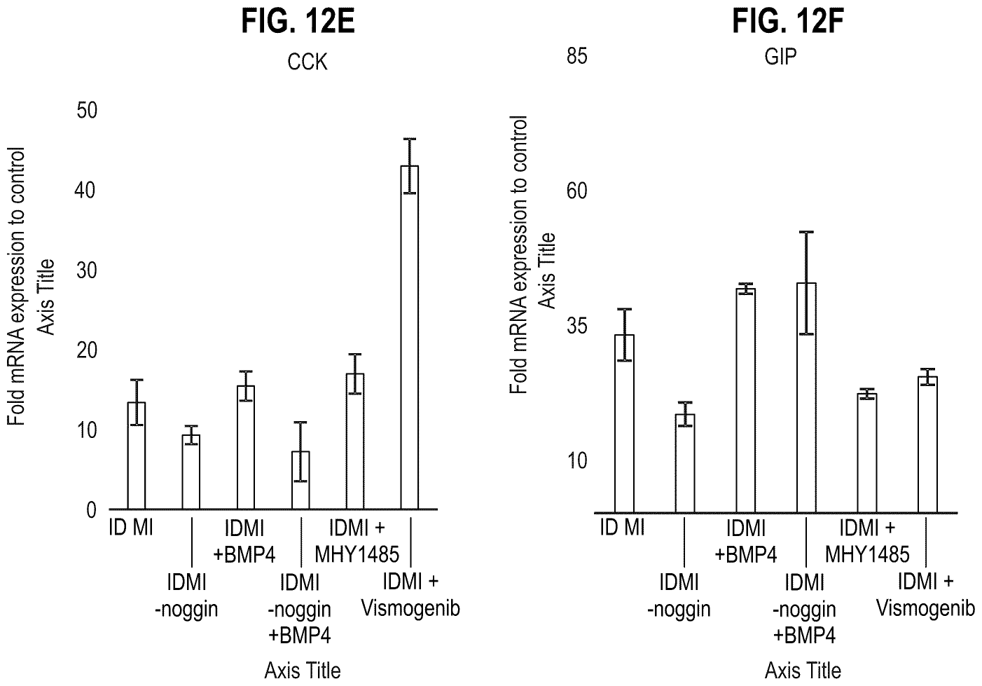

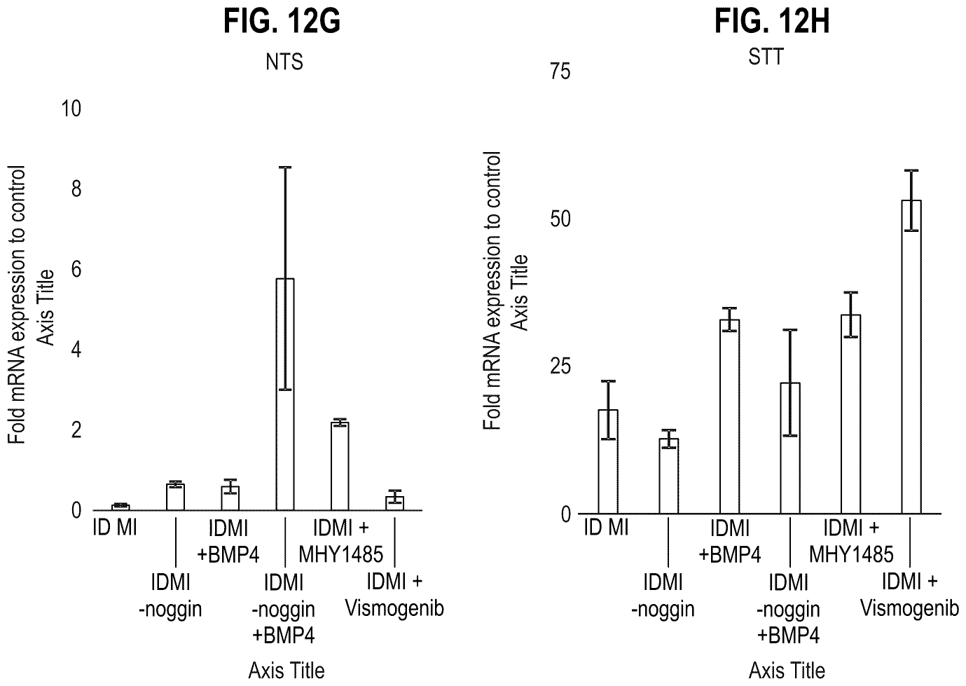

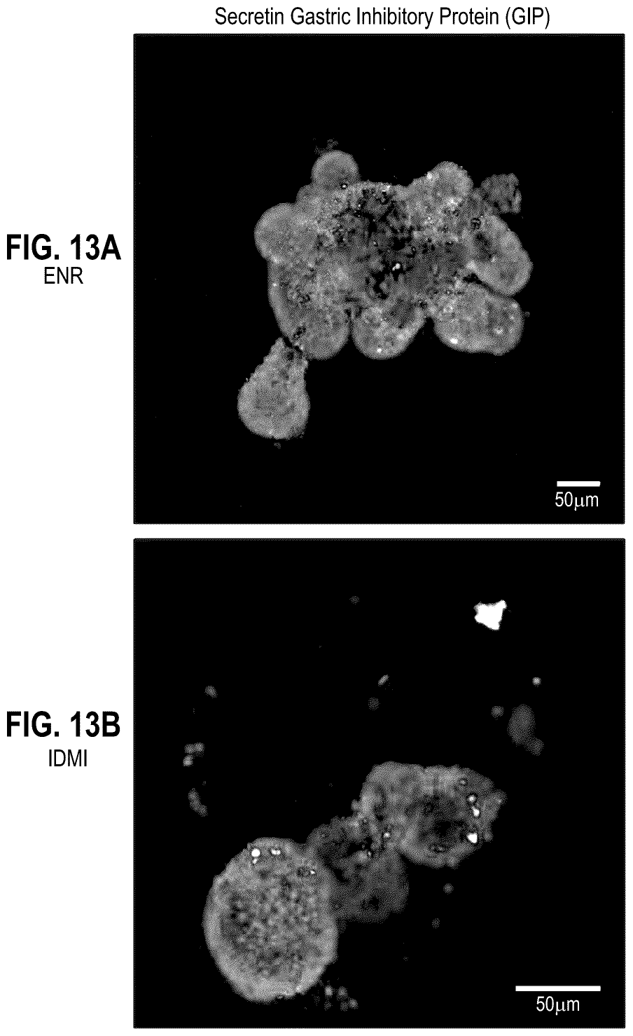

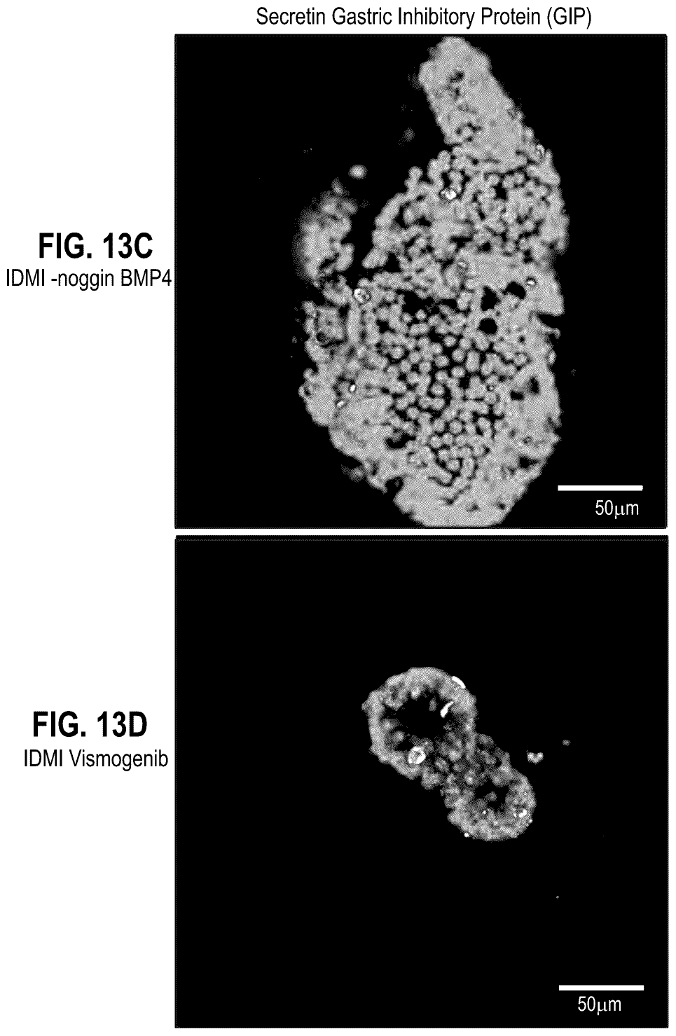

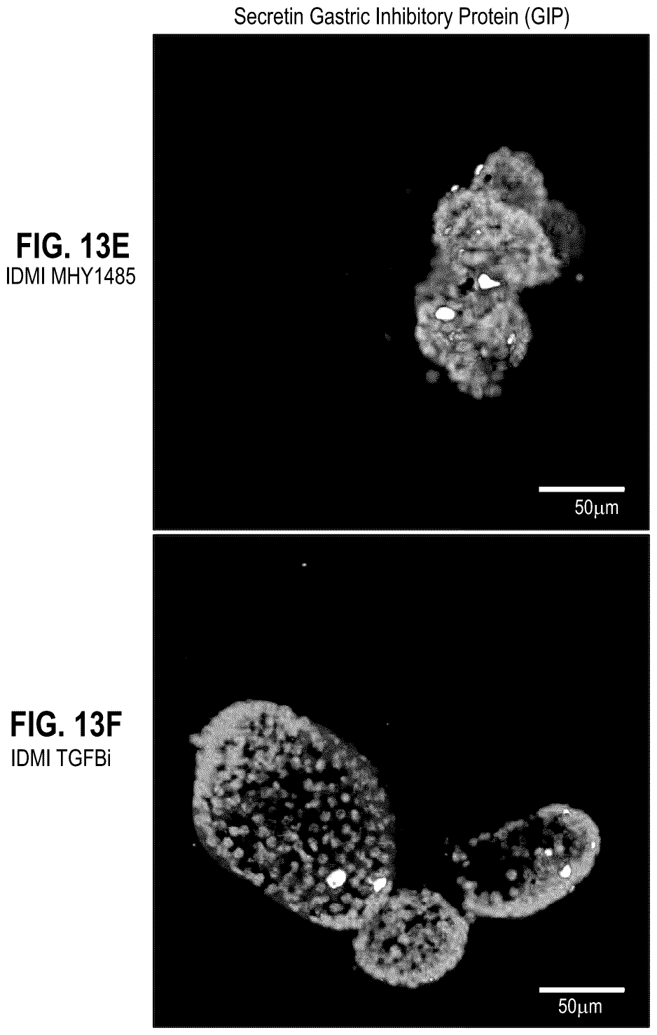

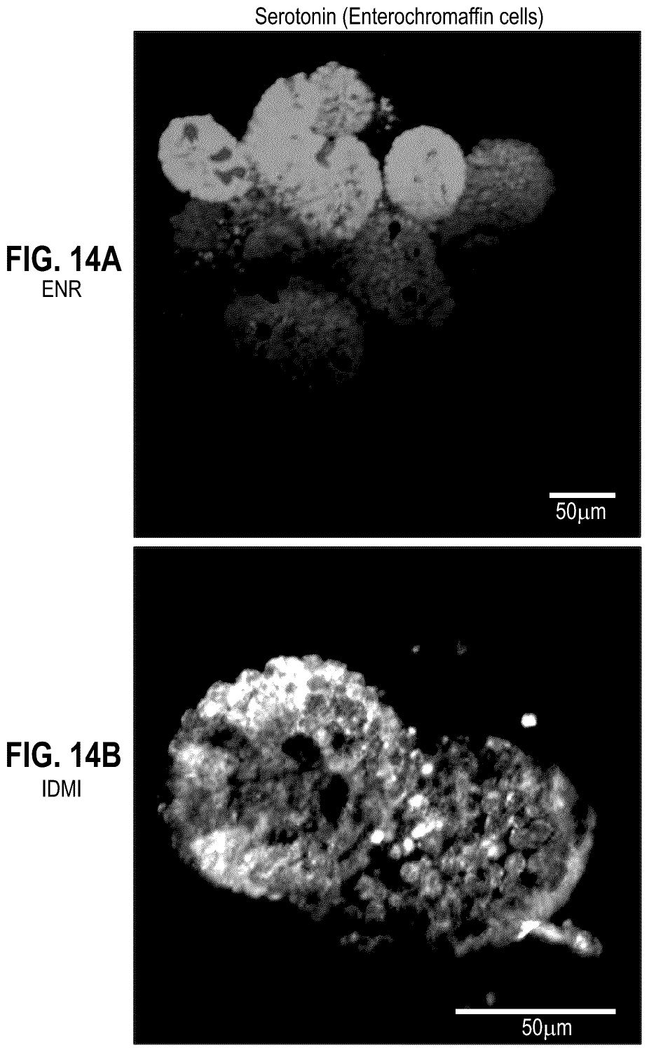

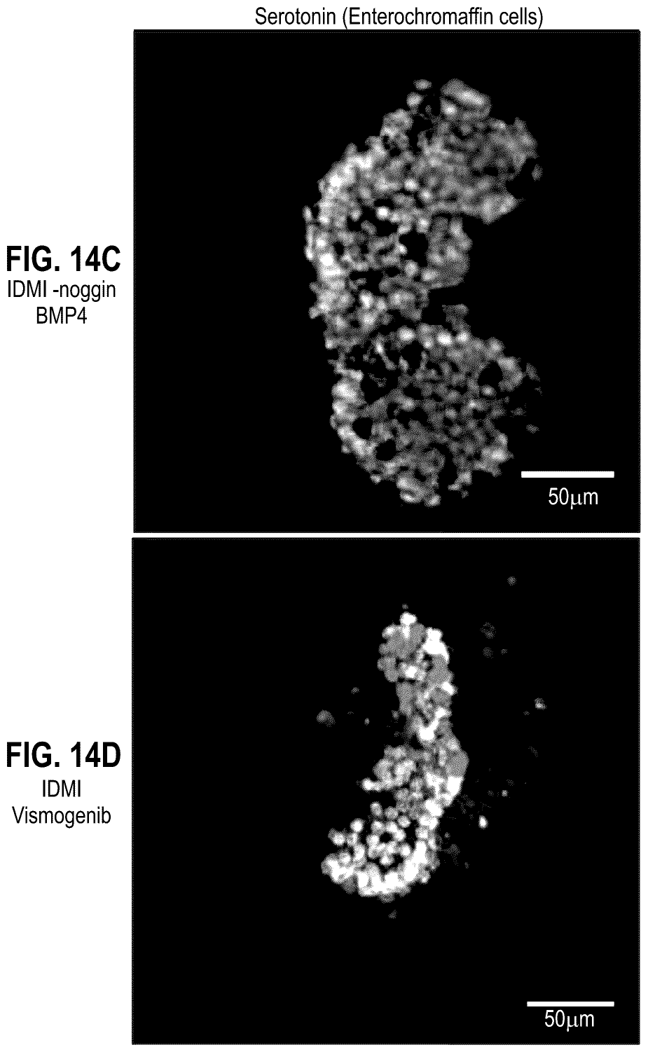

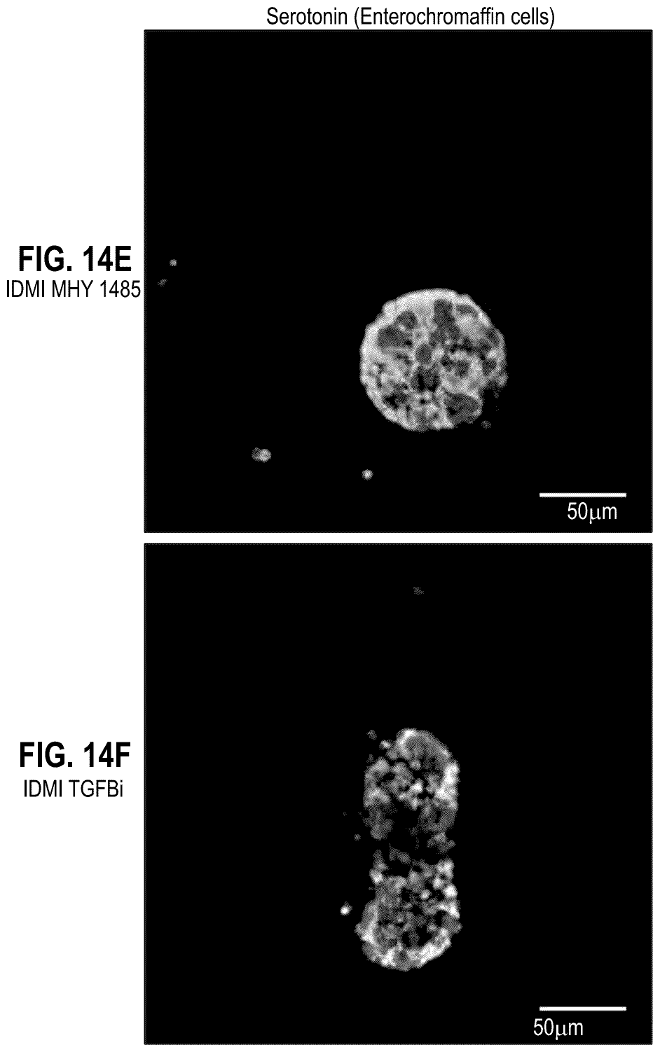

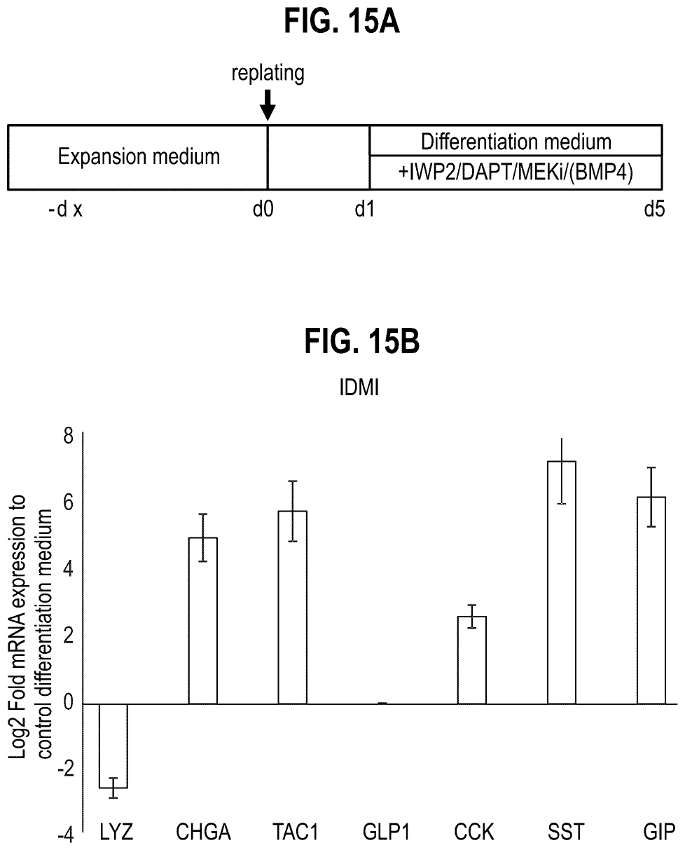

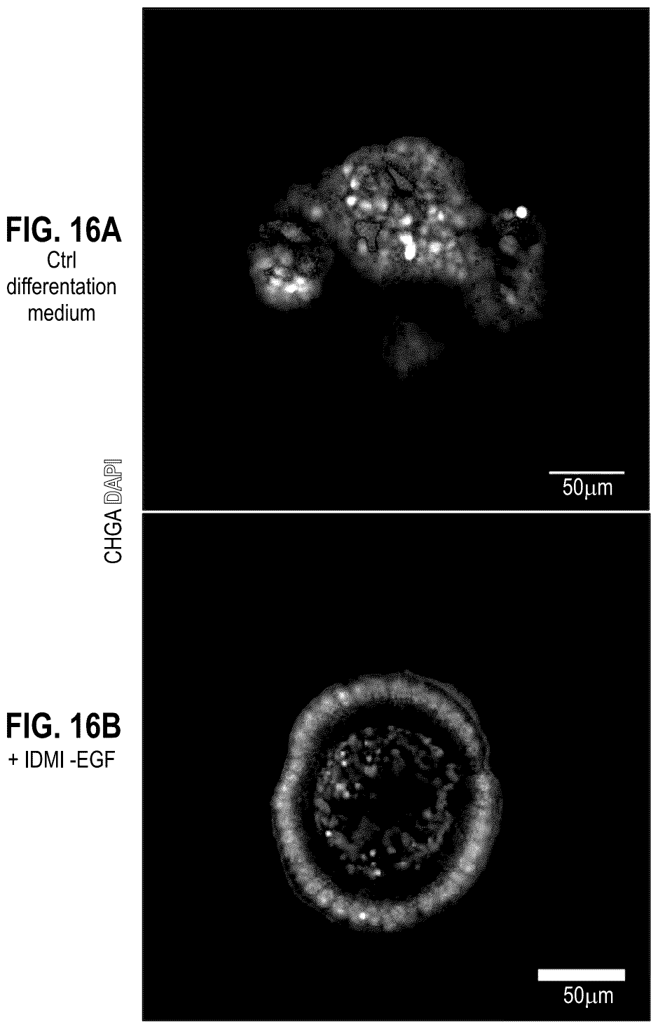

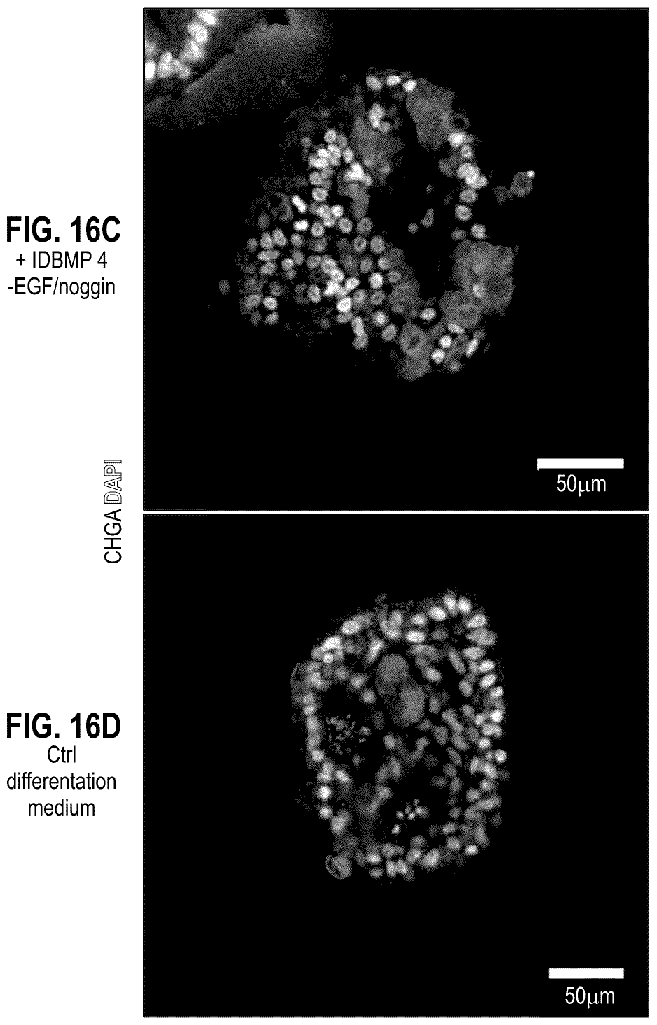

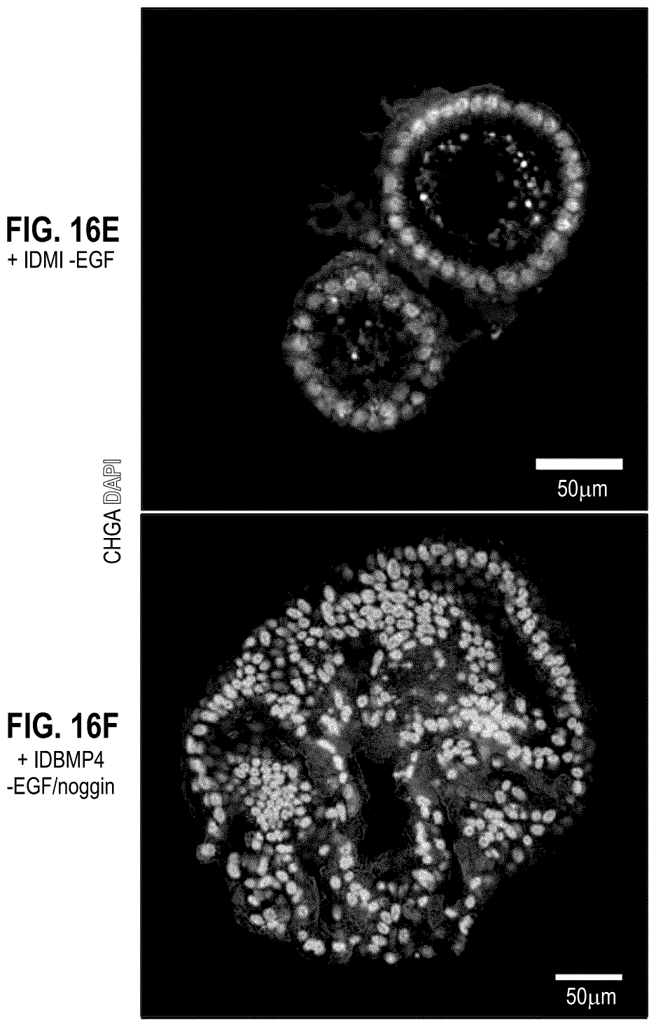

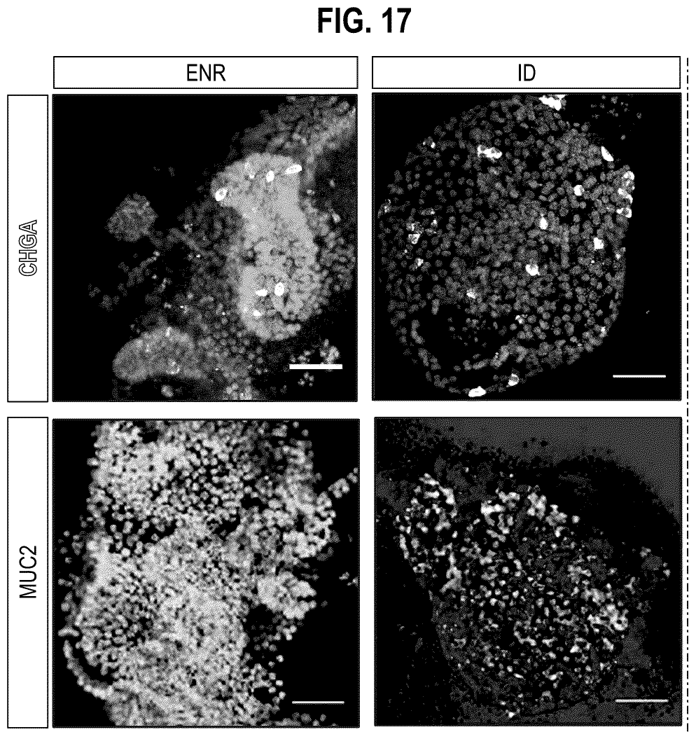

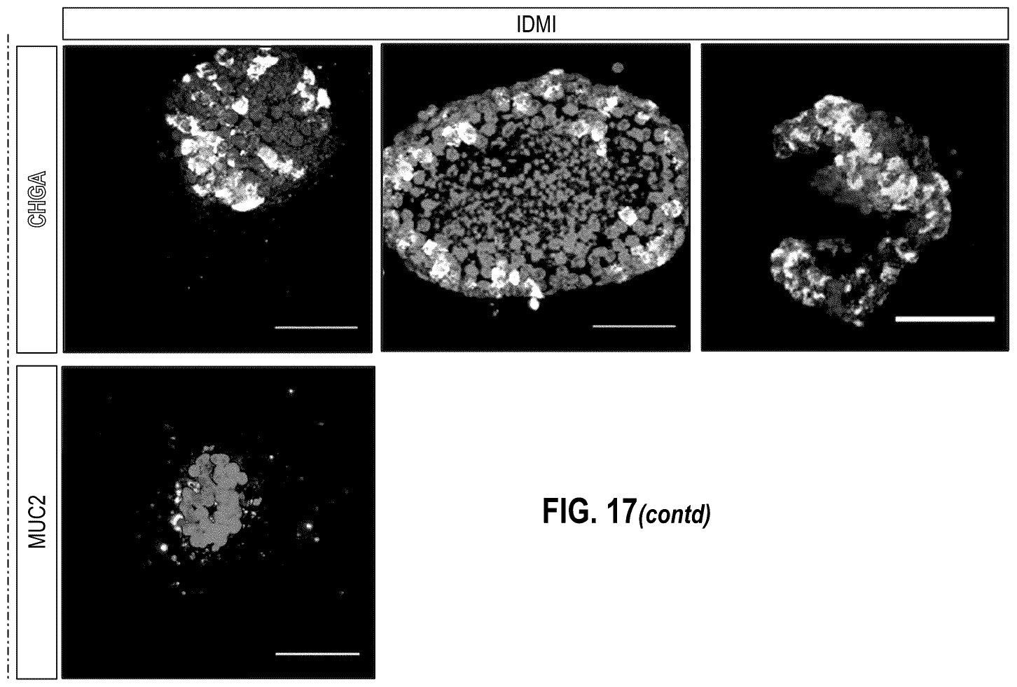

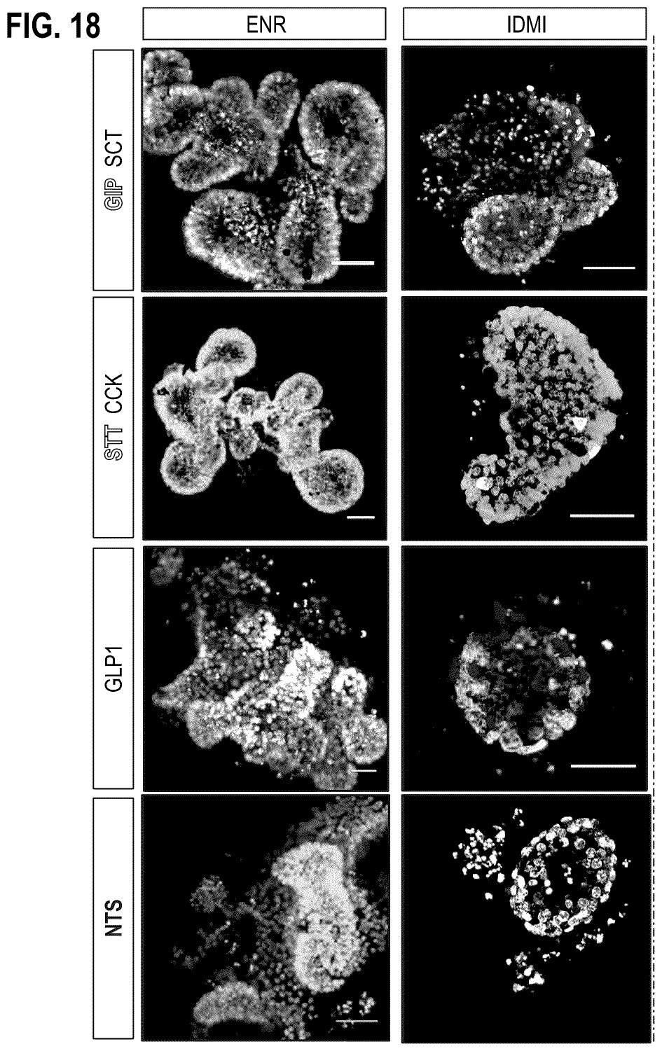

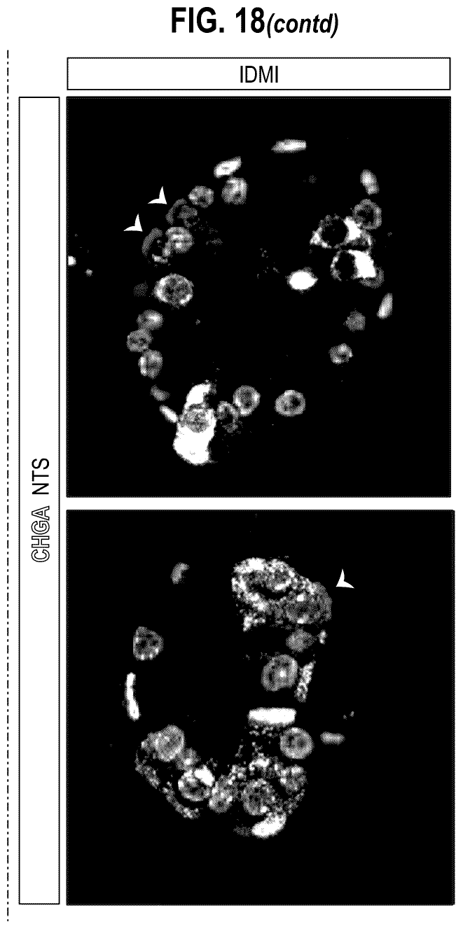

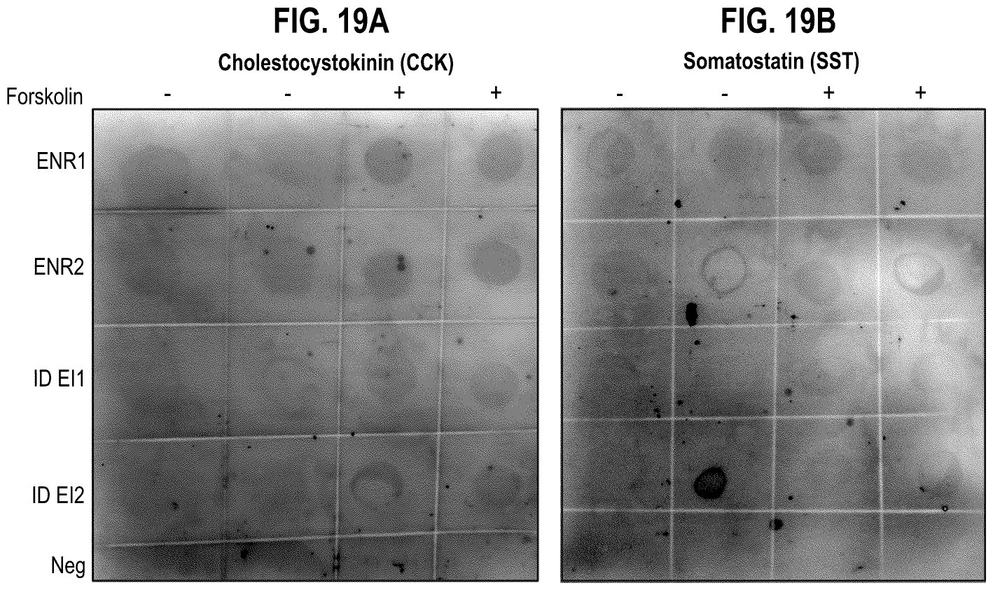

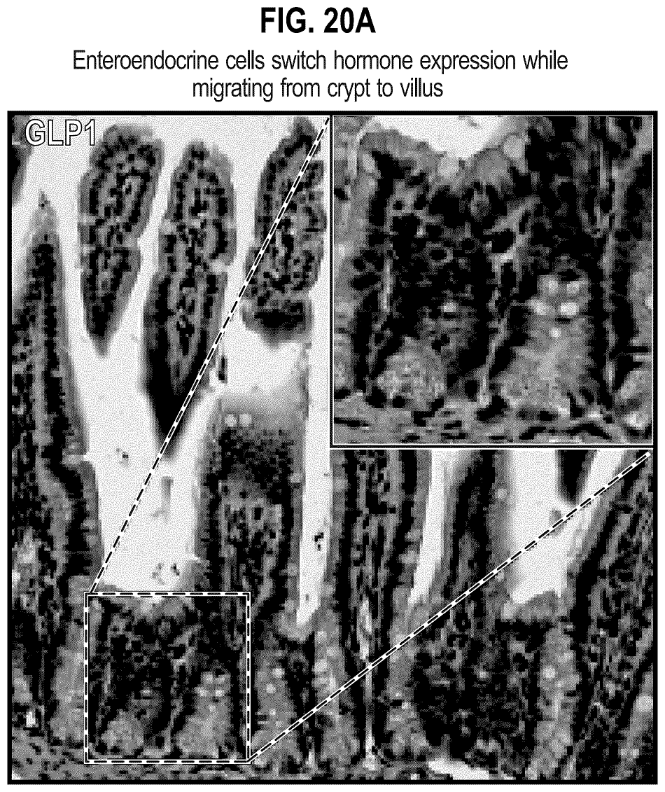

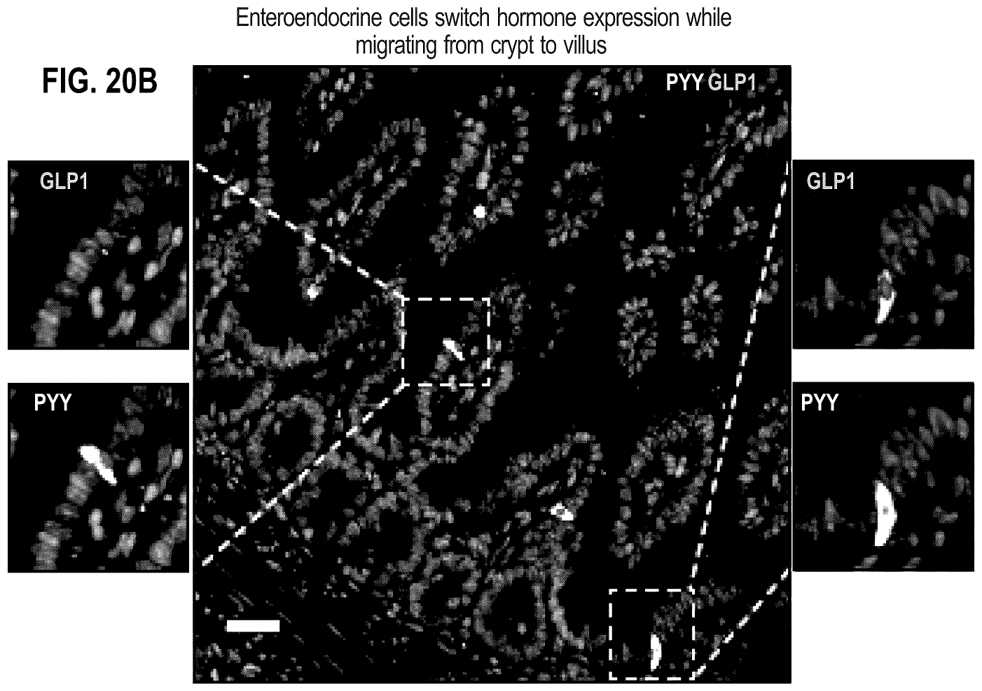

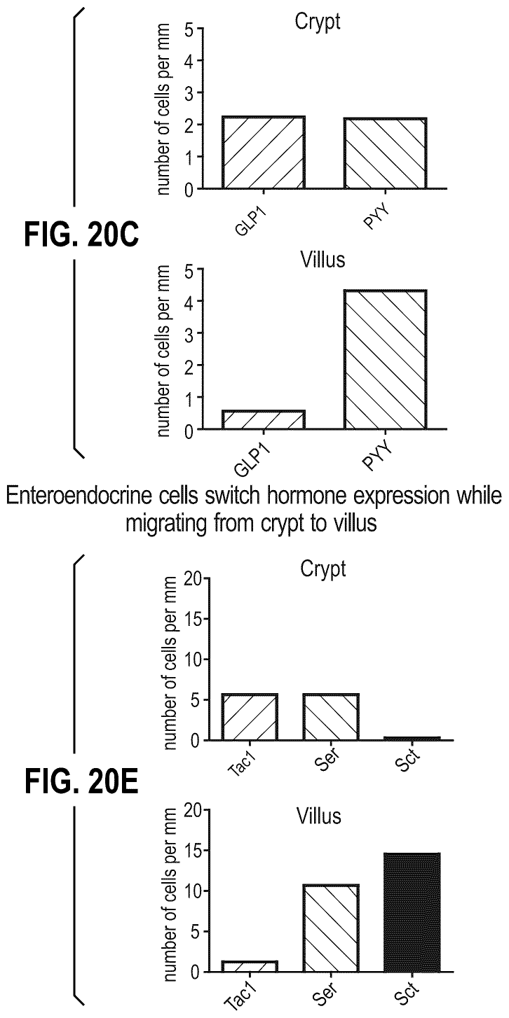

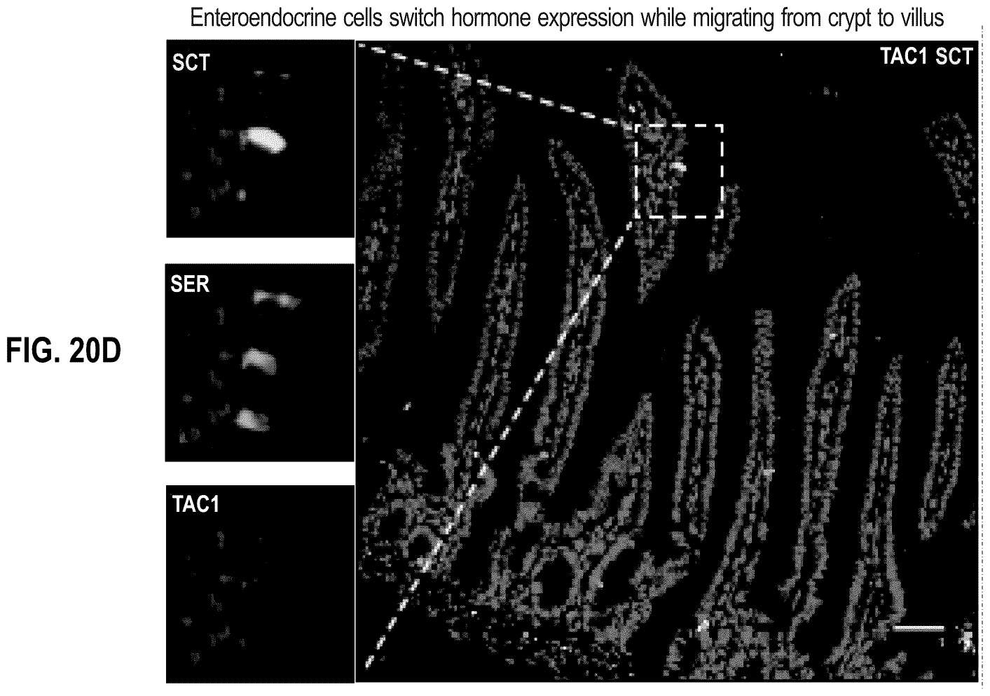

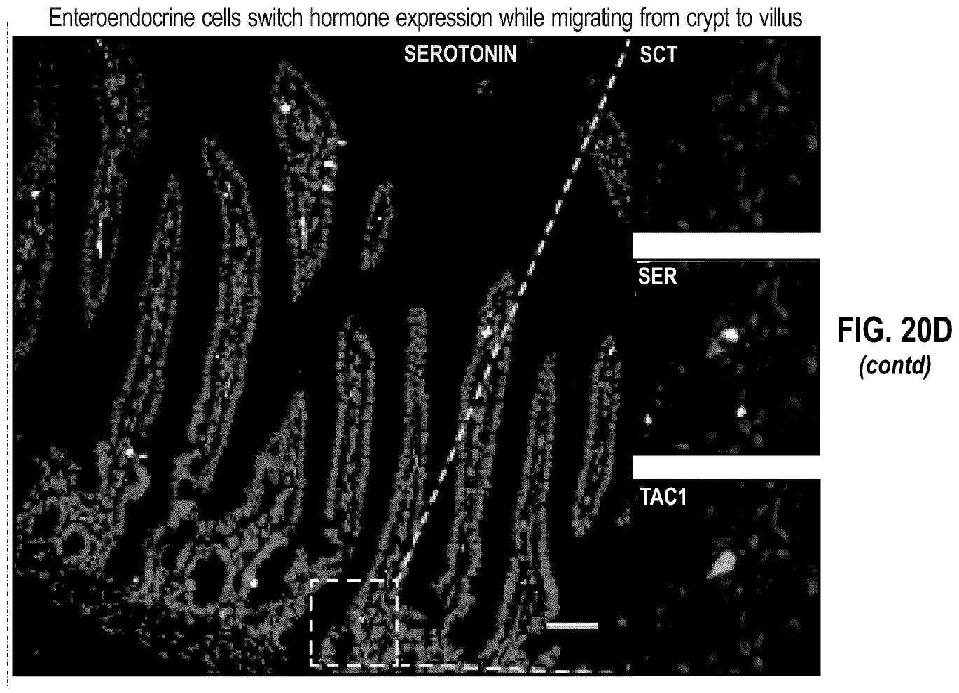

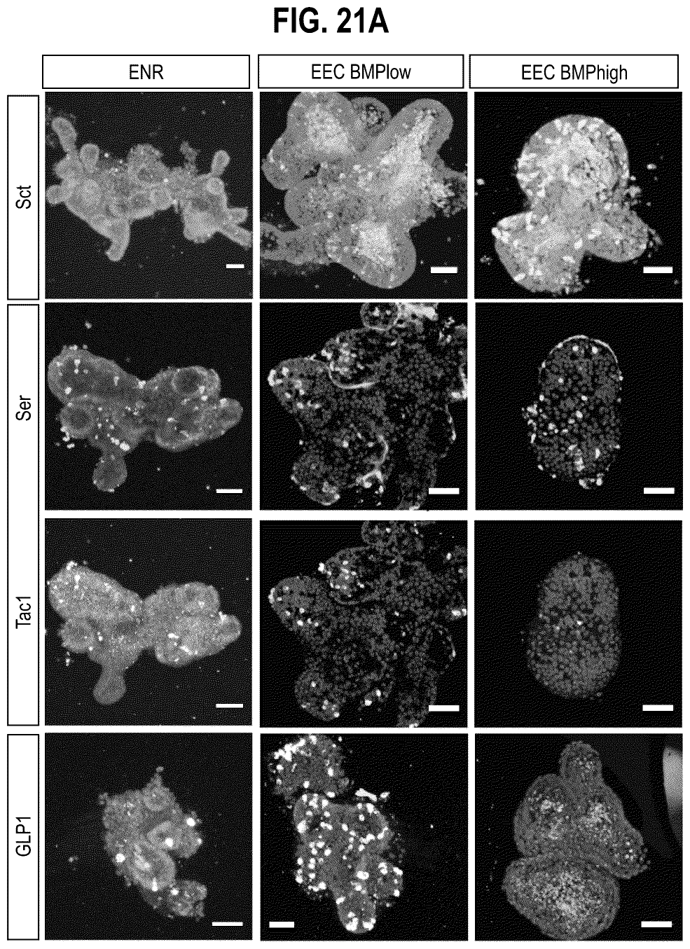

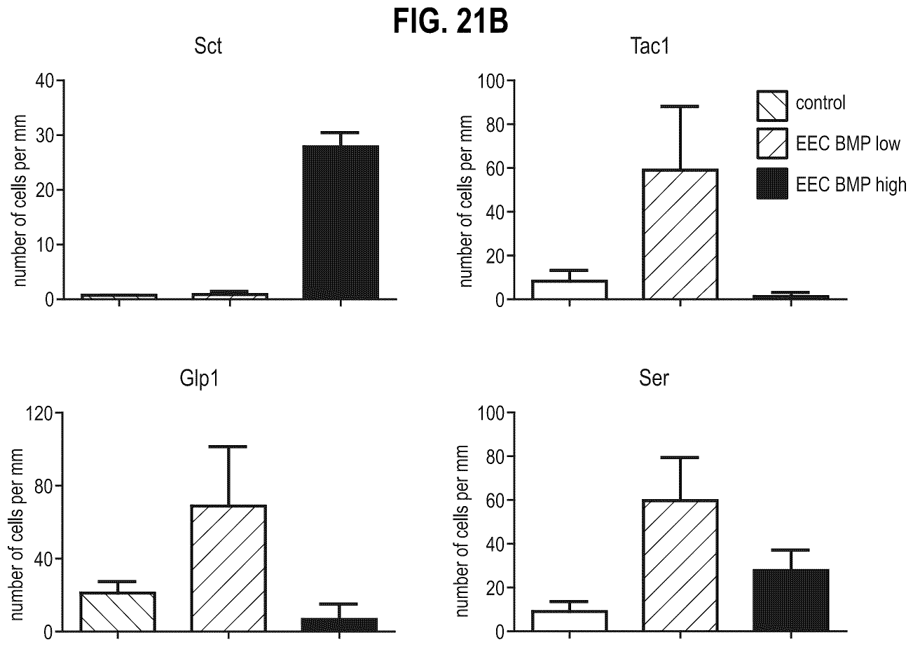

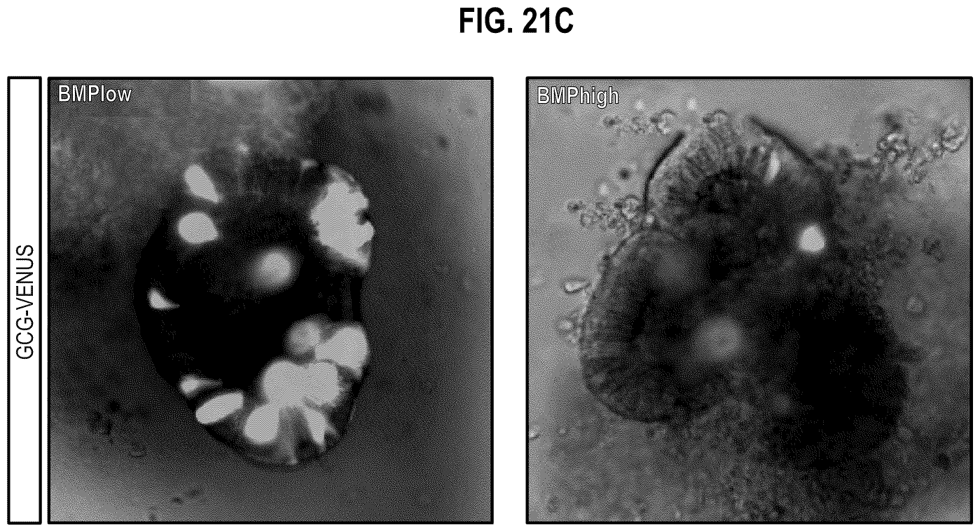

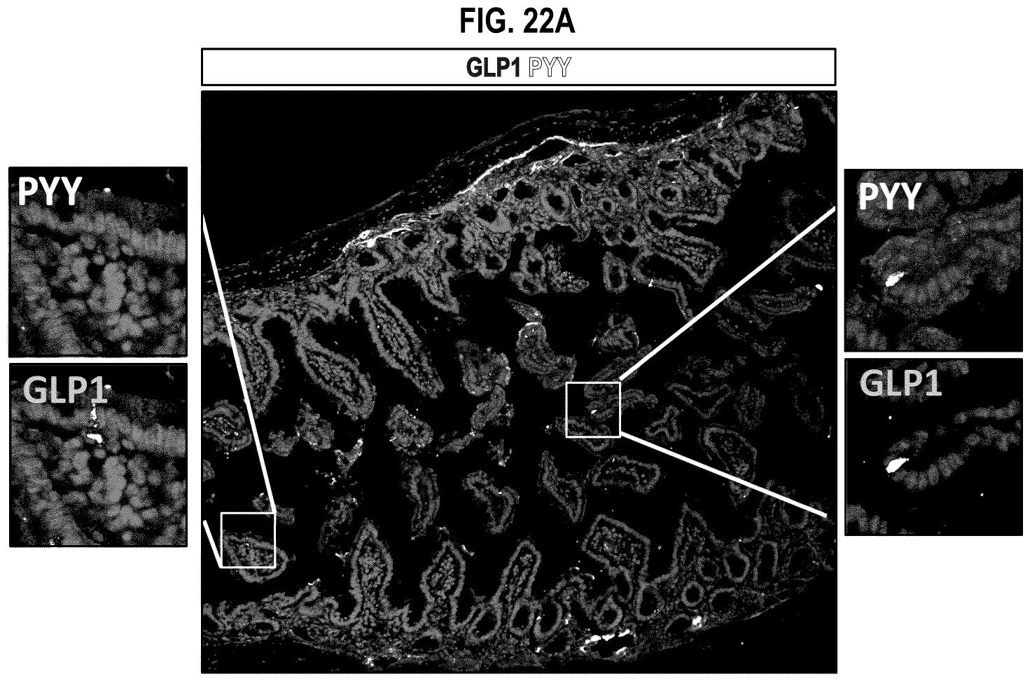

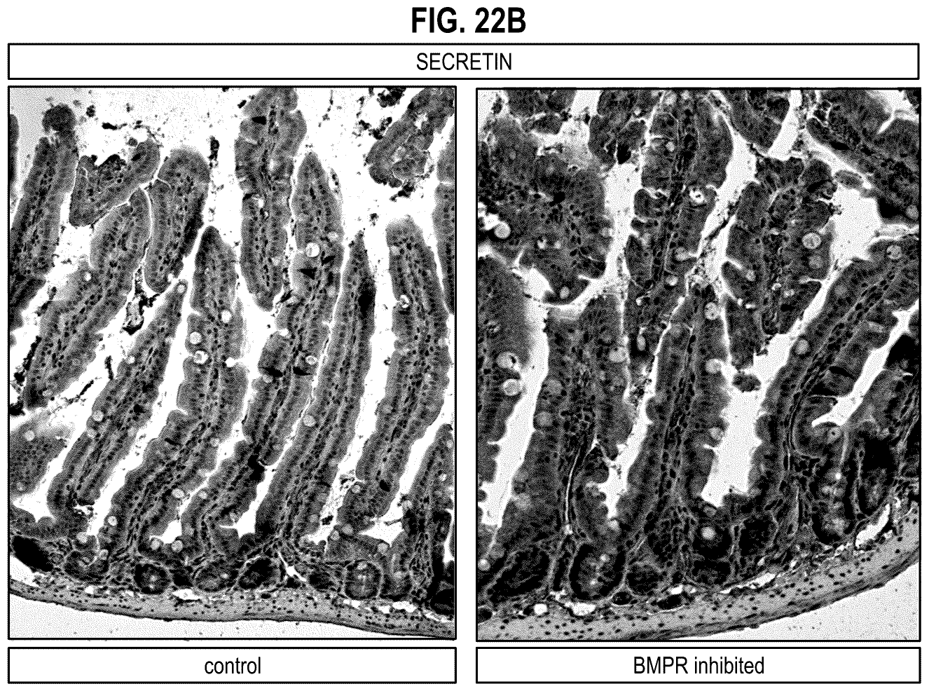

To elucidate the cellular composition of EEC induced organoids and the extent of heterogeneity in hormonal expression of individual ECCs, we performed single-cell RNA sequencing analysis. We sorted live single cells (without additional markers) from IDEGFRi and IDMeki treated organoids. Among the 289 cells that passed our filtering we identified a cluster of 94 cells as enterocytes enriched in Aldob (4.9×, p-adj<0.001), Apoa1 (12.6×, p-adj<0.001) and Alpi (5.6×, p-adj<0.001, FIG. 10). These were excluded from further analysis to better characterize remaining populations. Cells derived from both IDEGFRi or IDMeki treated organoids were distributed similarly in t-SNE space and were analyzed together (Figure S4).

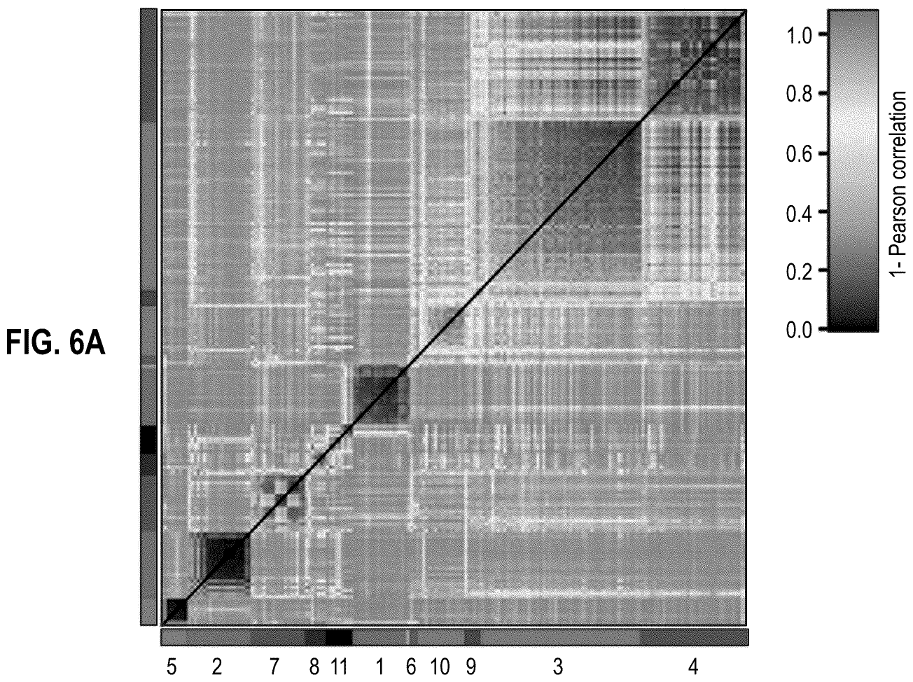

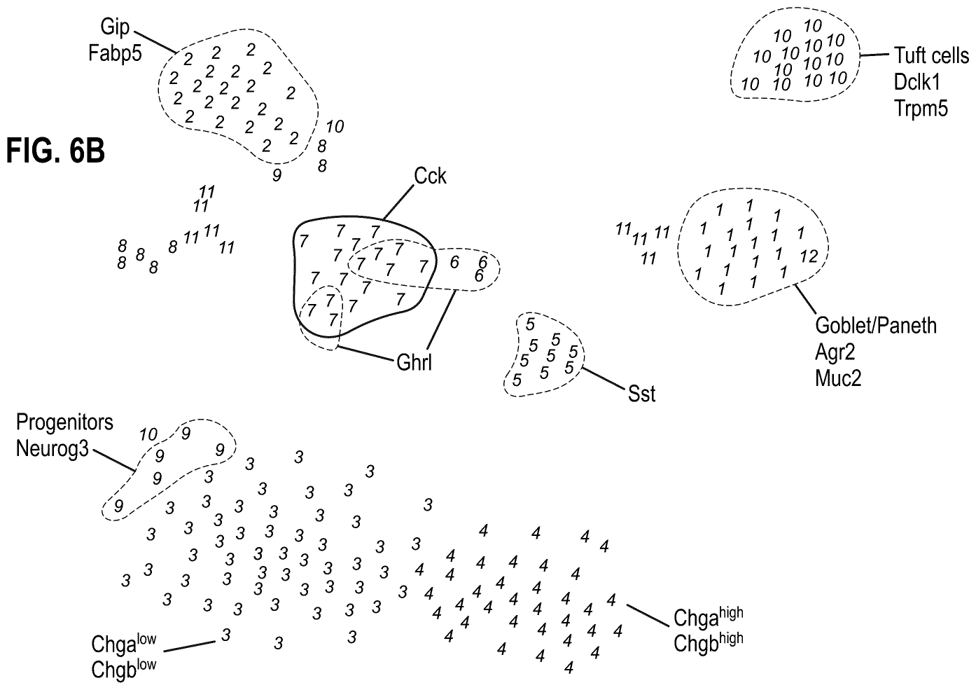

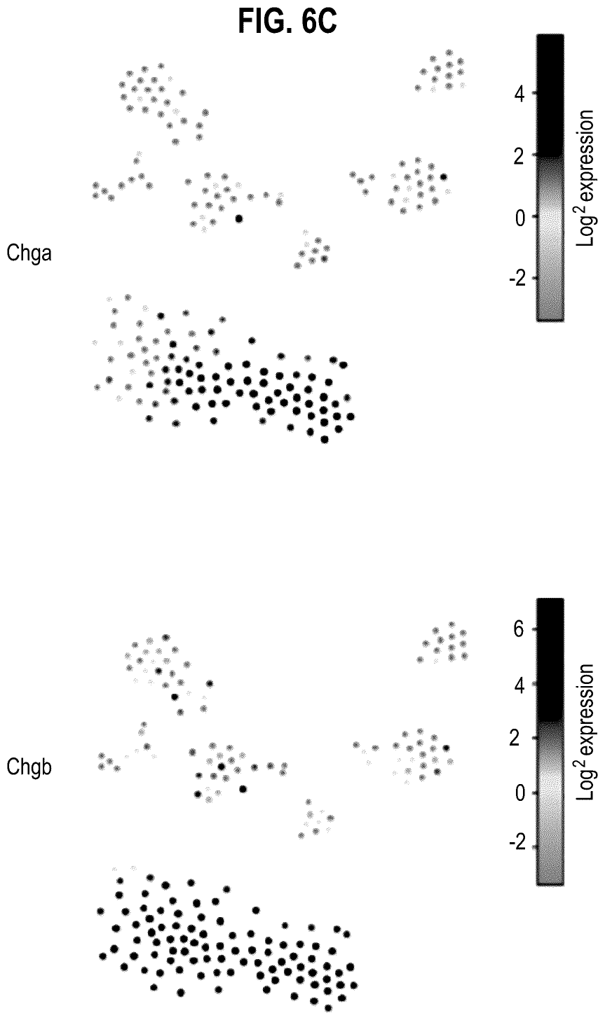

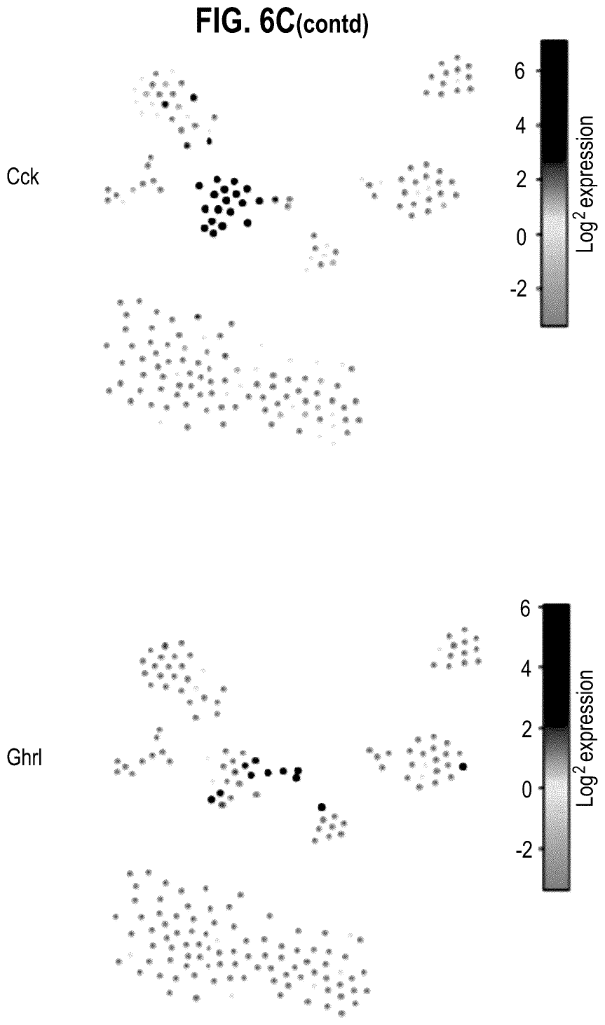

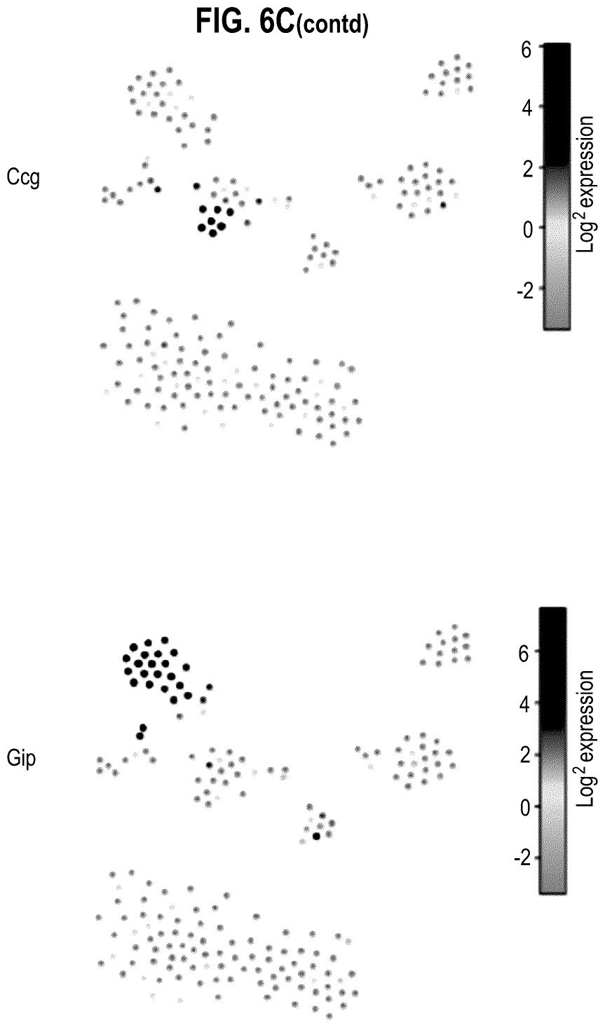

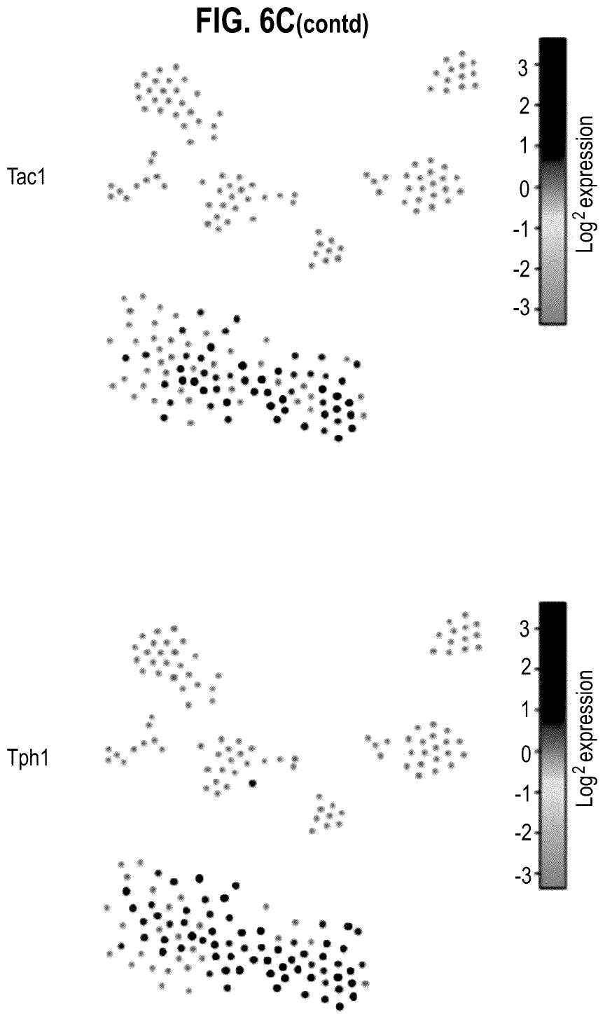

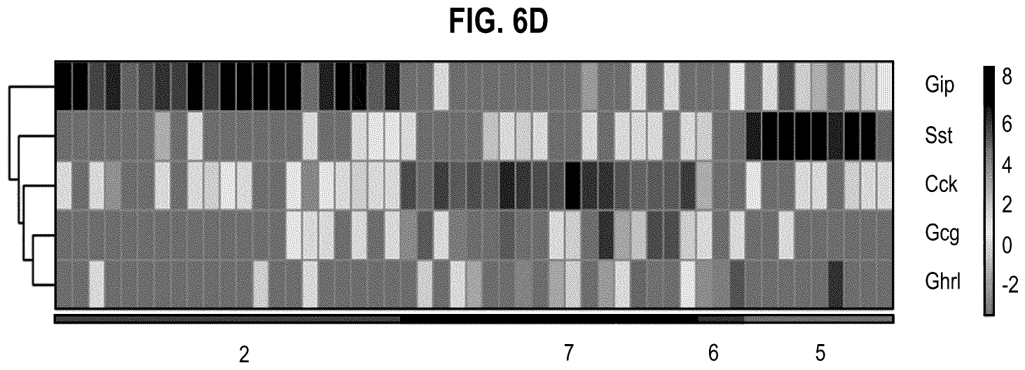

Using RaceID, we identified 12 distinct clusters of cells (FIG. 6A). k-means clustering of the Pearson correlation of cellular transcriptomes revealed a clear separation between clusters as well as possible heterogeneity within clusters (e.g. 7 and 8, FIG. 6A). Differential gene expression analysis revealed signature genes for each cluster, which we used to classify cell types (Table S1).

The most prominent clusters were 3 (53 cells) and 4 (35 cells) that expressed pan-EEC markers Chga and Chgb (FIG. 6C). Chga and Reg4 expression formed a gradient, both being higher in Cluster 4. Hormonal production in these Chgb high clusters was best defined by Tac1 and Tph1 expression, both markers of Enterochromaffin cells. Tac1 encodes for the hormone Substance P while Tph1 encodes for the rate-limiting enzyme in Serotonin synthesis (Egerod et al. (2012) Endocrinology 153:5782-5795; Grun et al. (2015) Nature 525:251-255). Both may act as neurotransmitters exciting the connected enteric neurons (Latorre et al. (2016) Neuro gastroenterology and motility: the official journal of the European Gastrointestinal Motility Society 28(5):620-630).

The other clusters had relatively low levels of Chga and Chgb transcripts but included cells expressing hormones (FIG. 6C). Cluster 2 (21 cells) was marked by Gip expression (74×) that is expressed by K-cells. Fabp5 was also highly enriched in this cluster (12,6×), consistent with its role in Gip secretion (Shibue et al. (2015) American journal of physiology, endocrinology and metabolism 308:E583-591). Cluster 5 (9 cells) members expressed very high levels of Sst (182×) identifying them as D-cells (FIG. 6C). Ghrelin (Ghr1) expression was distributed to more than one cluster, but was highest in cluster 6 (19×, 3 cells). We also noticed that Islet1 (Isl-1, 9,7×) was coexpressed with Ghr1 in these cells. Islet1 plays an important role in cell fate specification and its loss leads to impaired glucose homeostasis (Terry et al. (2014) American journal of physiology, gastrointestinal and liver physiology 307:G979-991). Cells in cluster 7 (18 cells) all highly expressed Cck (55,7×), and subgroups in this cluster were also rich in other hormones such as Gcg (28,2×), Ghr1 (5,3×) and Pyy (11,4×). Consistently, I-cells are reported to co-express Cck with other hormones at varying levels (Egerod et al. (2012) Endocrinology 153:5782-5795).

One of the early inducers of EEC differentiation is Neurogenin-3 (Neurog3), which is followed by Neurod1. Neurog3 (5,2×) expression was highest in cluster 9 (6 cells) and in some cells of cluster 3 that were most similar to cluster 9. Virtually all EEC clusters expressed Neurod1. Given the temporal expression of these transcription factors, we suggest that cluster 9 represents EEC progenitors, which then through Neurod1 generate a panel of EECs. Cluster 1 (18 cells) was enriched in Goblet and Paneth cell related genes, such as Agr2 (33×), Muc2 (26×), Ttf3 (23×) and Defa24 (28×). Even after filtering, some remaining enterocyte-like cells expressing Aldob and Mt1/2 were visible (Cluster 8, 7 cells). These clusters may have been generated prior to the EEC induction, as we do not see an increase in their numbers following EGFR or Mek inhibition. Dclk1 and Trpm5 expression identified cluster 10 (15 cells) as Tuft cells (FIGS. 6B and 6C). In total, 145/289 cells (50% of all cells) analyzed were EECs or their progenitors, confirming high efficiency of our induction protocol.

Since multiple hormones can be co-expressed in the same cell, we further looked into the heterogeneity of hormone expression at a single cell level (FIG. 6D). We focused on cluster 2, 5, 6 and 7 where multiple hormones were expressed. 4 major groups based on Gip, Sst, Cck and Ghrelin expression were visible. Cck+ cells are further split into Gcg+Ghrl+, Gcg+Ghrl−, Gcg−Ghrl+ and Gcg−Ghrl− clusters, with some also co-expressing low levels of Sst and/or Gip. Transcriptomes of Sst+ cells were more homogenous, coexpressed low levels of Gip and Cck while one cell co-expressed Ghrl only. We have previously reported partial overlap between Cck+ and Tac1+ cells. Consistently, some of the Tac1+ cells in clusters 3 and 4 expressed low levels of Cck (FIGS. 6B and 6C).

Overall, our single cell analysis indicates that our protocol enriched for EECs to ˜50% of the culture based on marker gene expression. Moreover, we generated a panel of EEC subtypes including some rare cells with complex hormonal expression profiles.

### Example 3

A murine EEC differentiation medium containing inhibitors of Wnt, Notch and MAPK signalling was used to generate organoids. These organoids contained a mixture of all EEC subtypes. Enterochromaffin cells that produce serotonin were the most abundant cell type in these cultures. The mechanism by which different EEC subtypes are generated was investigated. This was with a view to expand the uses of the culture and to understand development of these cells. In a first screen of signalling pathways that might control EEC subtype specification, modulations of the BMP, Hedgehog and mTOR pathways were found to affect the relative ratios of the EEC subtypes. Secretin-producing S cells seem to be to be particularly rare during Wnt, Notch and MAPK inhibition. S cells normally reside in the proximal duodenum, the same location as organoids in this study were isolated from. Activation of the BMP pathway by withdrawing Noggin from the EEC differentiation medium, and adding BMP4 (10 g/ml) to the culture medium changed the relative abundance of EEC subtypes. A drastic increase in the number of S-cells (on messenger, 400-fold over control), as well as secretin levels per cell (based on IHC), was observed after activation of the BMP pathway. This increase in S-cells seems to go at the expense of the number of enterochromaffin cells, suggesting potential overlapping developmental pathways. See further discussion of the role of BMP signaling in Example 5.

### Example 4

Human small intestine (SI) organoids were cultured first in expansion medium (as described in Sato et al. (2011) Gastroenterology 141(5):1762-72) to increase stem cell number, and were then replated in a differentiation medium (expansion medium lacking Wnt conditioned medium, TGFβ-inhibitor, p38 inhibitor and Nicotinamide) to direct differentiation. Replating involved disruption of Matrigel (without dissociation of organoids) in cold medium, washing of the organoids with basal culture medium (PBS can also be used) and subsequent plating in fresh Matrigel (without dissociation of organoids). Organoids were then cultured in the differentiation medium for 1 day. On the next day, the EEC specific enteroendocrine differentiation protocol. This involved the addition of an EEC differentiation medium for 5 days, which was the same as standard differentiation medium (expansion medium lacking Wnt conditioned medium, TGFβ-inhibitor, p38 inhibitor and Nicotinamide) with the addition of 1.5 μM IWP2, 10 mM DAPT, 100 nM MEKi PD0325901. Much lower levels of MAPK inhibitors were required for the differentiation of these human cells to an EEC fate in this experiment than were required for the differentiation of the murine cells to an EEC fate in Example 3 (100-500 nM versus 1-5 μM in the murine system), possibly due to the absence of Paneth cells that produce EGF in the human system.

### Discussion

Here we identify EGF signalling as an indispensable driver of Lgr5 stem cell proliferation in organoids. Under conditions where Wnt signalling is untouched but EGF signalling is blocked, actively dividing Lgr5 stem cells convert into quiescent Lgr5 cells that retain expression of various Wnt target genes. This cellular state can be maintained for up to a week. Yet, the simple restoration of EGF signalling converts the quiescent cells back into their normal active stem cell state. Differential expression analysis revealed the loss of well-known proliferation-inducing transcription factors such as the Ets-like factors Etv4 and -5, suggesting that they play a key role in stem cell division.

Thus, chemical inhibition of EGFR signalling arrested Lgr5+ stem cells without affecting their stem cell potential. We believe that high-level Wnt signalling maintains this stem cell potential during induced quiescence. In organoids as well as in crypts, Lgr5+ cells are always the direct neighbors of the Wnt3-secreting Paneth cells (Sato et al. (2011) Nature 469:415-418). In this setting, Wnt3 does not diffuse over distances, but is loaded directly onto the Lgr5 stem cells (Farin et al. (2016) Nature 530:340-343). The quiescent Lgr5 stem cells remain juxtaposed to the Paneth cells in EGFRi treated organoids and are thus exposed to high local Wnt signals.

Indeed, 3 independent Wnt target gene alleles as well as gene expression analyses confirmed an increase in Wnt signalling upon EGFR inhibition. In sum, our results show that maintenance of stem cell fate requires Wnt but not EGF, whereas stem cell proliferation depends on the combination of Wnt and EGF.

Previous studies have identified quiescent cells close to the zone of differentiation at the ‘+4’ position with stem cell potential (Sangiorgi and Capecchi (2008) Nature Genetics 40:915-920; Takeda et al. (2011) Science 334:1420-1424; Yan et al. (2012) Proceedings of the National Academy of Sciences of the United States of America 109:466-471). We have reported the existence of Dll1+ precursors that exclusively generate secretory cells at this position (van Es et al. (2012) Nature cell biology 14:1099-1104). Using a histone label retention assay, Doug Winton's group identified a similar population with secretory differentiation potential. These label retaining cells share a signature with CBCs including the expression of Lgr5, but express good levels of some of the secretory lineage genes, such as Chga (Buczacki et al. (2013) Nature 495:65-69). Taken together, these secretory precursors represent transient states, yet can de-differentiate into stem cells when the need arises and can thus be considered facultative stem cells (Buczacki et al. (2013) Nature 495:65-69; van Es et al. (2012) Nature cell biology 14:1099-1104). A similar situation exists for the abundant enterocyte precursors in the crypt (Tetteh et al. (2016) Cell stem cell 18:203-213).

To our surprise, we noticed a slight bias of quiescent Lgr5 cells towards expression of EEC markers, such as Chga, which made them reminiscent of the Lgr5+ label-retaining cells identified by Doug Winton. EECs represent fewer than 1% of the cell of the intestinal epithelium, yet together form the largest endocrine organ in the human body in terms of cell number (Latorre et al. (2016) Neurogastroenterology and motility: the official journal of the European Gastrointestinal Motility Society 28(5):620-630). EECs have been proposed to act as sensors of luminal content such as nutrients and microorganisms, and regulate physiological responses like glucose sensitivity, gastric emptying, mood and appetite through the secretion of hormones (Janssen and Depoortere (2013) TEM 24:92-100). The functional study of EECs has been hampered by a lack of robust in vitro systems to generate these cells in large amounts. We have previously reported the directed differentiation of stem cells into secretory cells by Notch inhibition (van Es et al. (2005) Nature 435:959-963). Paneth cell formation requires active Wnt signalling combined with Notch inhibition (van Es et al. (2012) Nature cell biology 14:1099-1104; van Es et al. (2005) Nature 435:959-963), and combined inhibition of Wnt and Notch signalling generates mainly Goblet cells (van Es et al. (2005) Nature 435:959-963; Yin et al. (2014) Nature Methods 11:106-112). By combining inhibition of EGFR pathway with Wnt and Notch blockage, EECs of a diversity of types are efficiently formed in primary cell culture. EGFR signalling has been shown to be important for the production of Goblet cells (Heuberger et al. (2014) Proceedings of the National Academy of Sciences of the United States of America 111:3472-3477). We believe that simultaneous inhibition of enterocyte, Paneth and Goblet cell fate by inhibiting Notch, Wnt and EGFR signalling respectively, is the key to the generation of EECs.

Up to 20 subtypes of EECs can be distinguished based on the production of specific peptides. These cells exist at different frequencies in the proximal to distal GI tract. Certain EECs have been found to express several functionally related hormones both at transcript and protein level (Egerod et al. (2012) Endocrinology 153:5782-5795; Grun et al. (2015) Nature 525:251-255), although some of this might be an intermediate stage of maturation. We demonstrate the simultaneous generation of several principle subtypes of EECs, by single-cell transcriptome profiling. Confirming our previous work (Grun et al. (2015) Nature 525:251-255), we detect Chga-high and -low populations of EECs. The former is enriched in Tac1/Tph1 expressing EECs with some cells lowly expressing Cck. The latter is very diverse in hormonal expression. Cells with overall similar transcriptomes profiles cluster together, even though they can be subdivided into several classes based on hormonal expression. The wide distribution of hormonal expression levels suggests that separation between EEC subtypes is not clear-cut. It is tempting to speculate that their expression is at least in part stochastic and might even be temporally dynamic.

### Example 5

### Enteroendocrine Cells Alter Hormone Expression During Crypt to Villus Migration

Multiple Enteroendocrine cell hormones are differentially enriched in the intestinal crypt and villus. L-cells co-express GLP1 and PYY in the crypt, but mostly lack GLP1 expression in the villus (FIG. 20A-C). Enterochromaffin cells produce Serotonin along the whole crypt-villus axis, but selectively co-express Tac1 in the crypt and Secretin in the villus (FIG. 20D-E). This suggests that EECs can switch hormone expression during migration from the crypt to villus, and that there is a potential lineage relationship between crypt and villus EECs. However, an alternative explanation for this observation is that cells immunoreactive for Secretin or PYY in the villus are derived from an unrelated progenitor in the crypt, negative for Serotonin and Tac1 or GLP1. To show that such a transdifferentiation event can occur during the lifetime of EECs, we analyzed intestines from Tac1-Cre/Rosa26tdTomato mice. This reporter faithfully labels cells in the crypt that co-express Tac1 and Serotonin. Furthermore, Serotonin+ cells and the majority of Secretin+ cells in the villus are traced, while being negative for Tac1. Other hormones, including CCK, are not derived from a Tac1/Serotonin+ progenitor cell. These data suggest that individual EECs can express different sets of hormones in the crypt and villus. The majority of Secretin-producing cells is part of the Enterochromaffin lineage. The capacity of EECs to switch hormone expression implies that there might be fewer unrelated differentiation pathways of EECs than previously anticipated, but that rather alterations in the types of hormones produced by a limited number of cells create a plethora of EEC states.

### BMP Signaling Induces a Villus Hormone Signature in Enteroendocrine Cells

We next asked whether hormonal switches that occur from crypt to villus migration are part of a default maturation process in EECs, or that this is simply reflecting different niche signals where EECs are exposed to. Multiple morphogens are known to exist from crypt to villus, including ascending levels of BMP and Hedgehog, and descending levels of Wnt signals. The differentiation protocol for Enteroendocrine cells in the murine intestinal organoid system described in earlier examples is based primarily on the triple inhibition of the Wnt, MAPK and Notch signaling pathway. We used this differentiation medium and added stem cell factor Noggin to the differentiation cocktail, thus creating a BMP low environment. Strikingly, we observed that all Enterochromaffin cells in this culture always co-express Serotonin and Tac1, while Secretin expression was lacking. This culture thus reflects an EEC hormone signature reminiscent of the crypt state, implying that niche signals are dominant over temporally driven EEC maturation.

To address whether niche signals indeed might be orchestrating EEC hormone signatures, we used our previously defined EEC differentiation system as a starting point and modulated signaling pathway on the background of this cocktail. Vismogenib was added to inhibit Hedgehog signaling, Noggin was withdrawn to activate BMPR1a/BMPR2 signaling and ALK5/4/7 inhibitor A83 or TGF-beta1 added to modulate different TGF-beta family receptor pathways. Although Wnt is already limited in the EEC differentiation cocktail by Porcupine inhibitor IWP2, we removed R-spondin on top to get a more pronounced and rapid inhibition of Wnt. We used Sct and Gcg transcript levels as a proxy of the villus and crypt hormone signatures respectively, while ChgA was included as transcript that has constant expression from crypt to villus. Inhibition of Hedgehog signaling does not modulate any of the hormones assessed, consistent with its predominant role in the intestinal mesenchyme. A83 and TGF-beta1 both inhibited expression of all EEC hormones. R-spondin removal similarly impaired EEC differentiation of all hormones assessed. This is potentially related due to a rapid differentiation of stem cells into the absorptive lineage when Wnt signals are inhibited rather than choosing a secretory fate when losing Notch signals. Finally, withdrawal of Noggin during EEC differentiation induced expression of Sct while repressing Gcg transcription, consistent with a switch from a crypt to villus expression profile. We added exogenous BMP4 to this EEC differentiation cocktail to further amplify BMP signaling, and named this combination “EEC BMPhigh” compared to the “EEC BMPlow” medium as we have previously defined. Stimulating organoids with this “EEC BMPhigh” differentiation medium induces the production of cells immunoreactive for Secretin, as well as Enterochromaffin cells expressing Serotonin without Tac1. The GLP1+ cell numbers were greatly diminished in this regime (FIG. 21A-C), without obvious morphological changes in the organoids. Organoids established from different intestinal regions maintain their regional identity in terms of hormone signatures, with enrichment of GIP in the proximal SI and Pyy, Nts and Gcg in the distal SI. Other hormones or EEC markers that show homogenous distribution from crypt to villus, including ChgA, Tph1, CCK and duodenal GIP, are only mildly affected by different BMP levels. We do observe an upregulation of Pyy and Nts which are expressed highest in the villus. The effects of BMP are summarised in the table below. Collectively, these data point to a role of BMP signals in the induction of hormones that are associated with EECs in the villus.

| EEC BMPhigh (BMP activator) | EEC BMPlow (BMP inhibitor) |
|---|---|
| Villus-like EEC characteristics | Crypt-like EEC characteristics |
| High secretin expression | Low secretin expression |
| Low Tac1 and Gcg expression | High Tac1 and Gcg expression |
| Low numbers of GLP1+ cells | High numbers of GLP1+ cells |

### Example 6

LDN193189 (Selleckchem Catalog. No.S2618) at 35 mg/kg was administered by oral gavage to mice in a 60 hour treatment. The LDN193189 was dissolved in citric acid at pH 3.1 and the oral gavage was given twice daily. This resulted in an expansion of GLP1 expressing cells in the villus of the intestine compared to control mice. Secretin expression was greatly reduced in cells in the villus of the intestine compared to control mice (see FIG. 22). The dose used is at the high end and it is anticipated that lower doses would also be effective. It is hypothesised that these results can be extrapolated to humans. LDN193189 is a selective BMP signaling inhibitor that inhibits the transcriptional activity of the BMP type I receptors ALK2 and ALK3 with IC50 of 5 nM and 30 nM in C2C12 cells, respectively, exhibits 200-fold selectivity for BMP versus TGF-β. Other inhibitors of BMP signalling, such as those described herein, are also envisaged to have similar effects in vivo. It is concluded that BMP inhibitors (and conversely BMP activators) have potential for use in therapy where modulation of GLP1 and/or secretin in vivo is desirable. Examples include, but are not limited to, treatment of diabetes, obesity, hypochlorhydria or hyperchlorhydria.

### Structure of LDN193189:

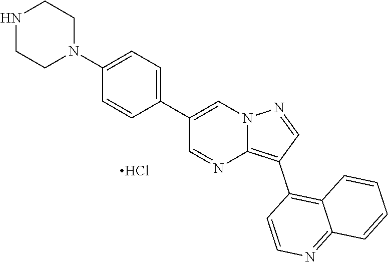 [SMILES: c1ccc2c(-c3cnn4cc(-c5ccc(N6CCNCC6)cc5)cnc34)ccnc2c1 ; Cl]

1. A method for differentiating progenitor cells, wherein said method comprises:
culturing the cells in a differentiation medium comprising a basal medium and further comprising one or more EGFR pathway inhibitors, a Notch inhibitor and one or more Wnt inhibitors.

2. The method of claim 1, wherein:
a. the one or more EGFR pathway inhibitors:
i. is selected from: (1) an EGFR inhibitor, (2) an EGFR and ErbB2 inhibitor, (3) an inhibitor of the RAS-RAF-MAPK pathway, (4) an inhibitor of the PI3K/AKT pathway and (5) an inhibitor of the JAK/STAT pathway; and/or
ii. comprises:
A. an EGFR inhibitor, such as Gefitinib:
B. an EGFR and ErbB-2 inhibitor, such as Afatinib; and/or
C. an inhibitor of the RAS-RAF-MAPK pathway, e.g. a MEK inhibitor, such as PD0325901, and/or an ERK inhibitor, such as SCH772984;
b. the Notch inhibitor is a gamma-secretase inhibitor, optionally DAPT or dibenzazepine (DBZ) or benzodiazepine (BZ) or LY-411575; and/or
c. the one or more Wnt inhibitors is selected from: (1) an inhibitor of Wnt secretion, (2) a competitive or non-competitive inhibitor of the interaction between Wnt or Rspondin and the Wnt receptor complex, (3) an inhibitor that promotes the degradation of components of the Wnt receptor complex, (4) an inhibitor of Dishevelled family proteins, (5) an activator that promotes destruction complex activity, (6) an inhibitor of the deoligomerisation of the destruction complex and/or (7) an inhibitor of β-catenin target gene expression, optionally wherein the one or more Wnt inhibitors comprise an inhibitor of Wnt secretion, e.g. a Porc inhibitor selected from IWP 2, LGK974 and IWP 1.

3.-9. (canceled)

10. The method of claim 1, wherein the differentiation medium further comprises:
a. one or more components selected from the group consisting of: a p38 inhibitor, a TGF-beta inhibitor, gastrin, a glucocorticoid, a receptor tyrosine kinase ligand, a BMP pathway activator, a cAMP pathway activator, a Hedgehog activator, a Hedgehog inhibitor, a modulator of mTOR signalling, B27 and N2;
b. one or more components selected from the group consisting of: a p38 inhibitor, a TGF-beta inhibitor, gastrin, a glucocorticoid, a receptor tyrosine kinase ligand, a BMP inhibitor, a cAMP pathway activator, a Hedgehog activator, a Hedgehog inhibitor, a modulator of mTOR signalling, B27 and N2;
c. a BMP activator, such as BMP7, BMP4 or BMP2; or
d. a BMP inhibitor, such as noggin, sclerostin, chordin, CTGF, follistatin, gremlin, tsg, sog, LDN193189 or dorsomorphin,
optionally, wherein the differentiation medium comprises less than 1 mM EGF.

11. A differentiation medium comprising a basal medium and further comprising one or more EGFR pathway inhibitors, a Notch inhibitor and one or more Wnt inhibitors.

12. A method for differentiating intestinal progenitor cells to obtain a population of intestinal cells enriched in enteroendocrine cells, wherein said method comprises:
culturing the intestinal progenitor cells in a differentiation medium according to claim 11.

13. A method for culturing epithelial stem cells, wherein said method comprises:
culturing the epithelial stem cells in the presence of an expansion medium for epithelial stem cells to obtain expanded epithelial stem cells; and subsequently
culturing the one or more expanded cells in a differentiation medium according to claim 11.

14. The method of claim 1, wherein:
a. the cells are cultured in contact with an extracellular matrix;
b. the method further comprises obtaining and/or isolating a differentiated cell population or a differentiated organoid;
c. the progenitor cells are epithelial cells, for example, from the intestine, stomach, pancreas, liver, prostate, lung, breast, ovary, salivary gland, hair follicle, skin, oesophagus, bladder, ear or thyroid;
d. the progenitor cells are from the intestine, stomach, pancreas or lung; and/or
e. the progenitor cells are mammalian progenitor cells, for example, human progenitor cells.

15.-18. (canceled)

19. A method for culturing intestine epithelial stem cells, preferably to obtain a differentiated intestine organoid, and wherein said method comprises:
culturing one or more intestine epithelial stem cells in contact with an extracellular matrix in the presence of an expansion medium; preferably wherein the expansion medium comprises a basal medium, and further comprises: a receptor tyrosine kinase ligand, a BMP inhibitor and a Wnt agonist and, optionally, valproic acid and a GSK-3 inhibitor (e.g. CHIR99021); and subsequently
culturing the one or more expanded intestine epithelial stem cells in contact with an extracellular matrix in the presence of a differentiation medium according to claim 11.

20. An organoid obtainable or obtained by a method of claim 1.

21. The organoid of claim 20, wherein the organoid is derived from the liver, pancreas, intestine, stomach, prostate, lung, breast, ovarian, salivary gland, hair follicle, skin, oesophagus, bladder, ear or thyroid, preferably from the intestine, stomach, pancreas or lung.

22. An organoid according to claim 20, wherein the organoid is derived from:
a. a human, and in which at least 30%, at least 40%, at least 50%, at least 60%, at least 70%, at least 80%, at least 90% or at least 99% of the cells express enteroendocrine cell markers; or
b. a mouse, and in which at least 30%, at least 40%, at least 50%, at least 60%, at least 70%, at least 80%, at least 90% or at least 99% of the cells express enteroendocrine cell markers;
optionally wherein the enteroendocrine cell markers are selected from Chga, Chgb, Tac1, Tph1, Gip, Fabp5, Ghr1, Pyy, Nts, Neurod1, Sst, Sct, cholecystokinin, glucagon and/or pro glucagon.

23. (canceled)

24. (canceled)

25. A composition comprising an organoid according to claim 20, and a differentiation medium comprising a basal medium and further comprising one or more EGFR pathway inhibitors, a Notch inhibitor and one or more Wnt inhibitors.

26. (canceled)

27. A method of treating a disorder, condition or disease comprising transplanting the organoid according to claim 20, or a cell derived from said organoid into a patient.

28. (canceled)

29. (canceled)

30. A method for screening for a therapeutic or prophylactic pharmaceutical drug or cosmetic, wherein the method comprises:
contacting a differentiated organoid according to claim 20 with a candidate molecule (or a library of candidate molecules),
evaluating said organoid for any effects (e.g. any change in the cell, such as a reduction in or loss of proliferation, a morphological change and/or cell death) or a change in organoid (e.g. the organoid size or motility);
identifying the candidate molecule that causes said effects as a potential drug or cosmetic; and optionally
preparing said candidate molecule as pharmaceutical or cosmetic.

31.-35. (canceled)

36. A method of obtaining a population of cells enriched in EECs, wherein the method comprises culturing a population of cells in a differentiation medium according to claim 11.

37. The method of claim 36, wherein:
a. the population of cells is enriched in GLP1-secreting EECs, and wherein the differentiation medium comprises a BMP inhibitor; or
b. the population of cells is enriched in secretin-secreting EECs, wherein the differentiation medium comprises a BMP pathway activator.

38. (canceled)

39. A method of:
a. treating or preventing diabetes mellitus or an associated disease or disorder; or
b. treating hyperchlorhydria or obesity,
wherein the method comprises administering a therapeutically effective amount of a BMP inhibitor to a subject in need thereof.

40. (canceled)


SEQUENCE LISTING (28 sequences)

SEQ ID NO: 1 - DNA, 20 bp, synthetic construct
  source 1..20: mol_type = other DNA
ccagctgcctgtagtgtcaa

SEQ ID NO: 2 - DNA, 20 bp, synthetic construct
  source 1..20: mol_type = other DNA
tcttgaggctcgccttgatg

SEQ ID NO: 3 - DNA, 21 bp, synthetic construct
  source 1..21: mol_type = other DNA
gccaagagccatgtgactatc

SEQ ID NO: 4 - DNA, 22 bp, synthetic construct
  source 1..22: mol_type = other DNA
cagagctggtactttggtgttc

SEQ ID NO: 5 - DNA, 20 bp, synthetic construct
  source 1..20: mol_type = other DNA
acccgccagtctcctacatc

SEQ ID NO: 6 - DNA, 20 bp, synthetic construct
  source 1..20: mol_type = other DNA
gcatctaggcgcagggattg

SEQ ID NO: 7 - DNA, 18 bp, synthetic construct
  source 1..18: mol_type = other DNA
gctgtgcaagctgaaggg

SEQ ID NO: 8 - DNA, 18 bp, synthetic construct
  source 1..18: mol_type = other DNA
gctgtgcaagctgaaggg

SEQ ID NO: 9 - DNA, 20 bp, synthetic construct
  source 1..20: mol_type = other DNA
cagctcgtccactctttccg

SEQ ID NO: 10 - DNA, 20 bp, synthetic construct
  source 1..20: mol_type = other DNA
cctctcgtctccttggaggg

SEQ ID NO: 11 - DNA, 20 bp, synthetic construct
  source 1..20: mol_type = other DNA
ggaatggatggctaccgtgg

SEQ ID NO: 12 - DNA, 20 bp, synthetic construct
  source 1..20: mol_type = other DNA
catgccacccatgctcgaat

SEQ ID NO: 13 - DNA, 21 bp, synthetic construct
  source 1..21: mol_type = other DNA
actaaggtggcctacctccaa

SEQ ID NO: 14 - DNA, 22 bp, synthetic construct
  source 1..22: mol_type = other DNA
ggaggtgacagtcaaggtgaga

SEQ ID NO: 15 - DNA, 20 bp, synthetic construct
  source 1..20: mol_type = other DNA
aggatccatctgtcctttgg

SEQ ID NO: 16 - DNA, 20 bp, synthetic construct
  source 1..20: mol_type = other DNA
acgttgtatgtcttggacag

SEQ ID NO: 17 - DNA, 20 bp, synthetic construct
  source 1..20: mol_type = other DNA
gaccccaagacactcagacg

SEQ ID NO: 18 - DNA, 20 bp, synthetic construct
  source 1..20: mol_type = other DNA
ttttctgtgtcctgctcgct

SEQ ID NO: 19 - DNA, 20 bp, synthetic construct
  source 1..20: mol_type = other DNA
cttcccagaagaagtcgcca

SEQ ID NO: 20 - DNA, 20 bp, synthetic construct
  source 1..20: mol_type = other DNA
gtgactggcacgagatgttg

SEQ ID NO: 21 - DNA, 20 bp, synthetic construct
  source 1..20: mol_type = other DNA
gaagagcggcgtatgtctgt

SEQ ID NO: 22 - DNA, 19 bp, synthetic construct
  source 1..19: mol_type = other DNA
ccagaaggagctttgcgga

SEQ ID NO: 23 - DNA, 20 bp, synthetic construct
  source 1..20: mol_type = other DNA
gacctgcgactagactgacc

SEQ ID NO: 24 - DNA, 20 bp, synthetic construct
  source 1..20: mol_type = other DNA
ccagttcctgtttcccggtg

SEQ ID NO: 25 - DNA, 20 bp, synthetic construct
  source 1..20: mol_type = other DNA
aactgttggctaggggacac

SEQ ID NO: 26 - DNA, 20 bp, synthetic construct
  source 1..20: mol_type = other DNA
tgatgaaagtcccctctgcg

SEQ ID NO: 27 - DNA, 20 bp, synthetic construct
  source 1..20: mol_type = other DNA
tgctgaccatcttccagctc

SEQ ID NO: 28 - DNA, 20 bp, synthetic construct
  source 1..20: mol_type = other DNA
gaatgtagggccttctgggt